(19) United States
(12) Patent Application Publication
(10) Pub. No.: US 2026/0263493 A1
(43) Pub. Date: Sep. 10, 2026

(54) METHODS FOR TREATMENT OF POST-SURGERY LAXITY OF TENDONS AND TENDON REPAIR

(71) Applicant: Nectero Medical, Inc., Tempe, AZ (US)

(72) Inventor: Kelvin Ning, Scottsdale, AZ (US)

(21) Appl. No.: 19/552,543

(22) Filed: Feb. 27, 2026

Related U.S. Application Data
(63) Continuation of application No. 18/177,019, filed on Mar. 1, 2023, now abandoned.
Application No. 18/177,019 is a continuation of application No. 17/448,450, filed on Sep. 22, 2021, now abandoned.
Application No. 17/448,450 is a continuation of application No. PCT/US2020/024731, filed on Mar. 25, 2020.
(60) Provisional application No. 62/824,034, filed on Mar. 26, 2019.
(60) Provisional application No. 62/824,033, filed on Mar. 26, 2019.
(60) Provisional application No. 62/824,119, filed on Mar. 26, 2019.
(60) Provisional application No. 62/824,160, filed on Mar. 26, 2019.
(60) Provisional application No. 62/824,187, filed on Mar. 26, 2019.
(60) Provisional application No. 62/824,020, filed on Mar. 26, 2019.
(60) Provisional application No. 62/824,031, filed on Mar. 26, 2019.
(60) Provisional application No. 62/824,058, filed on Mar. 26, 2019.
(60) Provisional application No. 62/824,044, filed on Mar. 26, 2019.
(60) Provisional application No. 62/824,105, filed on Mar. 26, 2019.

(51) Int. Cl. A61K 31/7034; A61K 9/06; A61K 47/10

(52) U.S. Cl. CPC A61K 31/7034; A61K 9/06; A61K 47/10

(57) ABSTRACT

Methods and compositions for treating post-surgery laxity of tendons or tendon repair are provided that utilize 1,2,3,4,6-pentagalloyl glucose (PGG) or analogues or derivatives thereof or LeGoo®. Also provided is a device to deliver 1,2,3,4,6-pentagalloyl glucose (PGG) or analogues or derivatives thereof or LeGoo® to the tissue to be treated.

### RELATED APPLICATIONS

This application is a continuation of U.S. application Ser. No. 18/177,019, filed Mar. 1, 2023, which is a continuation of U.S. patent application Ser. No. 17/448,450, filed Sep. 22, 2021, which is a continuation of PCT International Application No. PCT/US2020/024731 filed Mar. 25, 2020, which claims priority to U.S. Provisional Application Nos. 62/824,034, 62/824,033, 62/824,119, 62/824,160, 62/824,187, 62/824,020, 62/824,031, 62/824,058, 62/824,044, 62/824,105, all filed Mar. 26, 2019, the entire contents of each of which are hereby incorporated by reference herein.

### FIELD

Methods and compositions for treating laxity of tendons, particularly post-surgery laxity of tendons, are provided that utilize 1,2,3,4,6-pentagalloyl glucose (PGG) or analogues or derivatives thereof or LeGoo®. Also provided is a device to deliver 1,2,3,4,6-pentagalloyl glucose (PGG) or analogues or derivatives thereof or LeGoo® to the tissue to be treated.

### BACKGROUND

Tendon repair is surgery done to treat a torn or otherwise damaged tendon. Tendons are the soft, band-like tissues that connect muscles to bone. When the muscles contract, the tendons pull the bones and cause the joints to move. When tendon damage occurs, movement may be seriously limited. The damaged area may feel weak or painful. Tendon repair surgery may be helpful for people who have tendon injuries that are making it difficult for them to move a joint or are very painful.

### SUMMARY

Methods and compositions for treating laxity of tendons or ligaments, particularly post-surgery laxity of tendons, and for treatment of tendon and soft tissue attachment are provided that utilize 1,2,3,4,6-pentagalloyl glucose (PGG) or analogues or derivatives thereof or LeGoo®. Methods are also provided for treating peripheral vascular disease (e.g., peripheral arterial disease), chronic venous insufficiency, deep vein thrombosis, and varicose veins. Methods are also provided for treating stress urinary incontinence (SUI), pelvic organ prolapse, and congestive heart failure. Also provided is a device to deliver 1,2,3,4,6-pentagalloyl glucose (PGG) or analogues or derivatives thereof or LeGoo® to the tissue to be treated. For example, methods and devices for treating mitral valve disease are provided that utilize a weeping balloon catheter to deliver 1,2,3,4,6-pentagalloyl glucose (PGG) or analogues or derivatives thereof to surgical area of a removed native mitral valve, to a native mitral valve to be treated, or an implantation site of a replacement mitral valve, or to the site of transcatheter aortic valve replacement or implantation.

### Post-Surgery Laxity of Tendons and Tendon Repair

What is needed in the art are treatment protocols and compositions for stabilization of soft tissue such as tendons, particularly the laxity of post-surgery tendons.

In a first aspect, a method is provided for treating a post-surgery tendon laxity of a patient, comprising: exposing a tendon having post-surgical laxity of a patient; and delivering a therapeutic agent to the tendon, wherein the therapeutic agent comprises pentagalloyl glucose (PGG).

In an embodiment of the first aspect, the PGG is at least 99.9% pure.

In an embodiment of the first aspect, the therapeutic agent is substantially free of gallic acid or methyl gallate.

In an embodiment of the first aspect, the PGG is in admixture with a poloxamer gel.

In an embodiment of the first aspect, the delivering comprises spraying the tendon.

In an embodiment of the first aspect, the delivering comprises bathing the tendon.

In an embodiment of the first aspect, the delivering comprises injecting the tendon.

In a second aspect, a kit is provided for treating a post-surgery tendon laxity of a patient, comprising: a delivery device; and a therapeutic agent comprising pentagalloyl glucose (PGG); and a hydrolyzer.

In an embodiment of the second aspect, the PGG has a purity greater than or equal to 99%.

In an embodiment of the second aspect, the PGG is substantially free of gallic acid or methyl gallate.

In an embodiment of the second aspect, the PGG is in admixture with a poloxamer gel.

In an embodiment of the second aspect, the device is coated with the PGG.

In an embodiment of the second aspect, the hydrolyzer is ethanol.

In an embodiment of the second aspect, the hydrolyzer is dimethyl sulfoxide (DMSO) or contrast media.

In an embodiment of the second aspect, the kit further comprises a saline solution.

In a third aspect, a method is provided for preventing a post-surgery tendon laxity of a patient, comprising: administering pentagalloyl glucose (PGG) to a patient; and thereafter conducting a surgery associated with a risk of post-surgical tendon laxity.

In an embodiment of the third aspect, the PGG is at least 99.9% pure.

In an embodiment of the third aspect, the therapeutic agent is substantially free of gallic acid or methyl gallate.

In an embodiment of the third aspect, the PGG is in admixture with a poloxamer gel.

In an embodiment of the third aspect, the administering comprises spraying the tendon.

In an embodiment of the third aspect, the administering comprises bathing the tendon.

In an embodiment of the third aspect, the administering comprises injecting the tendon.

In an embodiment of the third aspect, the administering comprises systemically administering.

### Tendon and Soft Tissue Attachment in Surgical Repair

What is needed in the art are treatment protocols and compositions for stabilization of soft tissue during surgery, e.g., surgical tendon repair.

In a first aspect, a method is provided for treating soft tissue in connection with surgery, comprising: exposing soft tissue of a patient; and delivering a therapeutic agent to the soft tissue, wherein the therapeutic agent comprises pentagalloyl glucose (PGG).

In an embodiment of the first aspect, the PGG is at least 99.9% pure.

In an embodiment of the first aspect, the therapeutic agent is substantially free of gallic acid or methyl gallate.

In an embodiment of the first aspect, the PGG is in admixture with a poloxamer gel.

In an embodiment of the first aspect, the delivering comprises spraying the soft tissue.

In an embodiment of the first aspect, the delivering comprises bathing the soft tissue.

In an embodiment of the first aspect, the delivering comprises injecting the soft tissue.

In an embodiment of the first aspect, the soft tissue is muscle.

In an embodiment of the first aspect, the soft tissue is connective tissue.

In an embodiment of the first aspect, the soft tissue is skin.

In an embodiment of the first aspect, the soft tissue is nervous system tissue.

In an embodiment of the first aspect, the soft tissue is a blood vessel.

In an embodiment of the first aspect, the soft tissue is a fascial fiber.

In an embodiment of the first aspect, the soft tissue is a ligament.

In an embodiment of the first aspect, the soft tissue is a tendon.

In an embodiment of the first aspect, the soft tissue is in the oral cavity.

In a second aspect, a kit is provided for treating soft tissue in connection with surgery, comprising: a delivery device; and a therapeutic agent comprising pentagalloyl glucose (PGG); and a hydrolyzer.

In an embodiment of the second aspect, the PGG has a purity greater than or equal to 99%.

In an embodiment of the second aspect, the PGG is substantially free of gallic acid or methyl gallate.

In an embodiment of the second aspect, the PGG is in admixture with a poloxamer gel.

In an embodiment of the second aspect, the device is coated with the PGG.

In an embodiment of the second aspect, the hydrolyzer is ethanol.

In an embodiment of the second aspect, the hydrolyzer is dimethyl sulfoxide (DMSO) or contrast media.

In an embodiment of the second aspect, the kit further comprises a saline solution.

In an embodiment of the second aspect, the delivery device is a syringe or a catheter.

### Treatment of Peripheral Vascular Disease

In a first aspect, a method is provided of treating a peripheral vascular disease, comprising: administering, to a patient in need thereof, a composition comprising a compound of Formula:

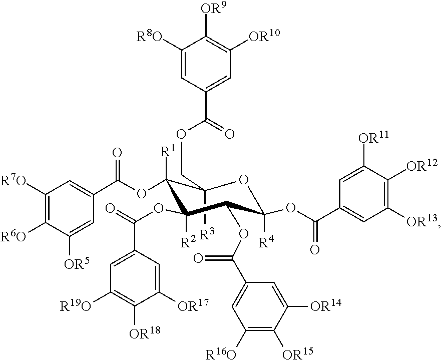

or a pharmaceutically acceptable salt thereof, wherein: R¹, R², R³ and R⁴ are each independently hydrogen or R^{A}; R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each independently hydrogen or R^{B}; each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, —NR^{Y}C(═X)OR^{X}, unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl; each R^{B} is independently selected from the group consisting of —C(═X)R^{Z}, —C(═X)N(R^{Y})₂, —C(═X)OR^{X}, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, or two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring; each X is independently oxygen (O) or sulfur (S); each R^{X} and R^{Y} is independently selected from the group consisting of hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl; and each R^{Z} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, and wherein the composition is substantially free of gallic acid or methyl gallate.

In an embodiment of the first aspect, at least one of R¹, R², R³ and R⁴ is R^{A}.

In an embodiment of the first aspect, at least two of R¹, R², R³ and R⁴ are R^{A}.

In an embodiment of the first aspect, each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, and —NR^{Y}C(═X)OR^{X}.

In an embodiment of the first aspect, each R^{A} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl.

In an embodiment of the first aspect, each R^{A} is independently selected from the group consisting of unsubstituted C₁₋₁₂alkoxy, unsubstituted C₁₋₈alkyl, unsubstituted C_{6 or 10}aryl, unsubstituted C₇₋₁₂aralkyl, unsubstituted 5-10 membered heteroaryl, unsubstituted C₃₋₁₂ heteroaralkyl, and unsubstituted 3-10 membered heterocyclyl.

In an embodiment of the first aspect, R¹, R², R³ and R⁴ are each hydrogen.

In an embodiment of the first aspect, R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each hydrogen.

In an embodiment of the first aspect, at least one of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ is R^{B}.

In an embodiment of the first aspect, at least two of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are R^{B}.

In an embodiment of the first aspect, at least three of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are R^{B}.

In an embodiment of the first aspect, each R^{B} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl.

In an embodiment of the first aspect, each R^{B} is independently selected from the group consisting of unsubstituted C₁₋₁₂alkoxy, unsubstituted C₁₋₈alkyl, unsubstituted C_{6 or 10}aryl, unsubstituted C₇₋₁₂aralkyl, unsubstituted 5-10 membered heteroaryl, unsubstituted C₃₋₁₂ heteroaralkyl, and unsubstituted 3-10 membered heterocyclyl.

In an embodiment of the first aspect, two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered. In an embodiment of the first aspect, substantially free is less than about 0.5% gallic acid.

In an embodiment of the first aspect, substantially free is less than about 0.5% methyl gallate.

In a second aspect, a method is provided for treating a peripheral vascular disease in a patient, comprising: delivering a therapeutic agent to a patient in need thereof, wherein the therapeutic agent comprises pentagalloyl glucose (PGG).

In an embodiment of the second aspect, the PGG is at least 99.9% pure.

In an embodiment of the second aspect, therapeutic agent is substantially free of gallic acid or methyl gallate.

In an embodiment of the second aspect, therapeutic agent further comprises one or more pharmaceutically-acceptable excipients.

In an embodiment of the second aspect, the method further comprises delivering at least one anticoagulant.

In an embodiment of the second aspect, the method further comprises delivering at least one member of the group consisting of heparin, enoxaparin, dalteparin, fondaparinux, warfarin, dabigatra, rivaroxaban, apixaban, and edoxaban.

In an embodiment of the second aspect, the method further comprises delivering a thrombolytic agent.

In an embodiment of the second aspect, the method further comprises delivering at least one member of the group consisting of reteplase, alteplase, urokinase, prourokinase, anisoylated purified streptokinase activator complex), and streptokinase.

In an embodiment of the second aspect, the PGG is administered systemically in an oral or intravenous form.

In an embodiment of the second aspect, the PGG is administered in topical form directly to a vessel to be treated or tissue adjacent to the vessel to be treated.

In an embodiment of the second aspect, the PGG is administered by injection into a treatment area or a vessel to be treated.

In an embodiment of the second aspect, the PGG is administered by a delivery catheter into a vessel to be treated.

In an embodiment of the second aspect, the PGG is admixed with a biocompatible poloxamer gel having reverse thermosensitive properties.

In a third aspect a device is provided for treatment of peripheral vascular disease, comprising: a balloon configured for administering angioplasty; and pentagalloyl glucose (PGG).

In an embodiment of the third aspect, the PGG has a purity greater than or equal to 99%.

In an embodiment of the third aspect, at least a portion of the balloon is coated with the PGG.

In an embodiment of the third aspect, at least a portion of the balloon is impregnated with the PGG.

In an embodiment of the third aspect, the balloon is configured for delivery of the PGG.

In an embodiment of the third aspect, the PGG is admixed with a biocompatible poloxamer gel having reverse thermosensitive properties.

In an embodiment of the third aspect, the balloon is configured to support a stent.

In an embodiment of the third aspect, the balloon is attached to a first end of a shaft and comprises a plurality of pores for delivering a therapeutic agent to a vessel to be treated.

In a fourth aspect, a kit is provided for treating a peripheral vascular disease, comprising: the device of the third aspect or any of its embodiments; pentagalloyl glucose (PGG); and a hydrolyzer.

In an embodiment of the fourth aspect, the PGG has a purity greater than or equal to 99%.

In an embodiment of the fourth aspect, the hydrolyzer is ethanol.

In an embodiment of the fourth aspect, the hydrolyzer is dimethyl sulfoxide (DMSO) or contrast media.

In an embodiment of the fourth aspect, the kit further comprises a saline solution.

In a fifth aspect, a catheter is provided for treating a peripheral vascular disease by angioplasty, the catheter comprising: an elongate body configured to be introduced into a treatment site of an occluded vessel, the elongate body having a proximal end, a distal end, and a main shaft having a lumen extending therethrough; and a first inflatable balloon coupled to the distal end of the elongate body, the first inflatable balloon having an interior volume in fluid communication with a first inflation lumen, wherein the first inflatable balloon circumferentially surrounds the elongate body, wherein the first inflatable balloon comprises a plurality of pores disposed on a surface of the first inflatable balloon configured to place the interior volume of the first inflatable balloon in fluid communication with the occluded vessel.

In an embodiment of the fifth aspect, the first inflatable balloon is further configured to support a stent.

In an embodiment of the fifth aspect, the first inflatable balloon is generally toroidal forming an annular interior volume that surrounds the elongate body.

In an embodiment of the fifth aspect, the pores are disposed on a central portion of the first inflatable balloon.

In an embodiment of the fifth aspect, the pores are disposed on a distal portion of the first inflatable balloon.

In an embodiment of the fifth aspect, the pores are not disposed on a proximal portion of the first inflatable balloon.

In an embodiment of the fifth aspect, the pores are not disposed on any portion of the first inflatable balloon proximal to a maximum expanded diameter of the balloon in an inflated configuration.

In an embodiment of the fifth aspect, the maximum expanded diameter of the first inflatable balloon is greater than the maximum expanded diameter of the first inflatable balloon.

In an embodiment of the fifth aspect, the catheter further comprises a second inflatable balloon disposed within the interior volume of the first inflatable balloon, the second inflatable balloon having an interior volume in fluid communication with a second inflation lumen.

In an embodiment of the fifth aspect, expansion of the second inflatable balloon is configured to at least partially expand the first inflatable balloon.

In an embodiment of the fifth aspect, expansion of the second inflatable balloon is configured to facilitate expulsion of at least a partial volume of inflation fluid disposed within the interior volume of the first inflatable balloon through the pores into the environment of the occluded vessel.

In a sixth aspect, a kit is provided for treating a peripheral vascular disease, comprising: the catheter of the fifth aspect or any of its embodiments; pentagalloyl glucose (PGG); and a hydrolyzer.

In an embodiment of the sixth aspect, the PGG has a purity greater than or equal to 99%.

In an embodiment of the sixth aspect, the hydrolyzer is ethanol.

In an embodiment of the sixth aspect, the hydrolyzer is dimethyl sulfoxide (DMSO) or contrast media.

In an embodiment of the sixth aspect, the kit further comprises a saline solution.

In a seventh aspect, a method is provided for treating an occluded blood vessel of a patient by angioplasty, comprising: positioning a first balloon in an occluded vessel; expanding the first balloon such that it forces the occluded vessel to open, with surfaces of the occluded vessel in contact with a surface of the first balloon; and delivering a therapeutic agent to the occluded vessel through pores in the first balloon.

### Treatment of Stress Urinary Incontinence

In a first aspect, a method is provided of treating stress urinary incontinence, comprising: administering, to a patient in need thereof, a composition comprising a compound of Formula:

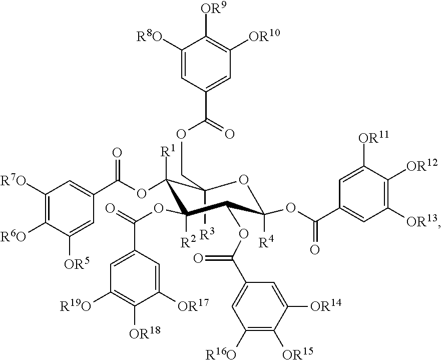

or a pharmaceutically acceptable salt thereof, wherein. R¹, R², R³ and R⁴ are each independently hydrogen or R^{A}; R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each independently hydrogen or R^{B}; each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, —NR^{Y}C(═X)OR^{X}, unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl; each R^{B} is independently selected from the group consisting of —C(═X)R^{Z}, —C(═X)N(R^{Y})₂, —C(═X)OR^{X}, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, or two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring; each X is independently oxygen (O) or sulfur (S); each R^{X} and R^{Y} is independently selected from the group consisting of hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl; and each R^{Z} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, and wherein the composition is substantially free of gallic acid or methyl gallate.

In an embodiment of the first aspect, at least one of R¹, R², R³ and R⁴ is R^{A}.

In an embodiment of the first aspect, at least two of R¹, R², R³ and R⁴ are R^{A}.

In an embodiment of the first aspect, each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, and —NR^{Y}C(═X)OR^{X}.

In an embodiment of the first aspect, each R^{A} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl.

In an embodiment of the first aspect, each R^{A} is independently selected from the group consisting of unsubstituted C₁₋₁₂alkoxy, unsubstituted C₁₋₈alkyl, unsubstituted C_{6 or 10}aryl, unsubstituted C₇₋₁₂aralkyl, unsubstituted 5-10 membered heteroaryl, unsubstituted C₃₋₁₂ heteroaralkyl, and unsubstituted 3-10 membered heterocyclyl.

In an embodiment of the first aspect, R¹, R², R³ and R⁴ are each hydrogen.

In an embodiment of the first aspect, R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each hydrogen.

In an embodiment of the first aspect, at least one of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ is R^{B}.

In an embodiment of the first aspect, at least two of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are R^{B}.

In an embodiment of the first aspect, at least three of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are R^{B}.

In an embodiment of the first aspect, each R^{B} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl.

In an embodiment of the first aspect, each R^{B} is independently selected from the group consisting of unsubstituted C₁₋₁₂alkoxy, unsubstituted C₁₋₈alkyl, unsubstituted C_{6 or 10}aryl, unsubstituted C₇₋₁₂aralkyl, unsubstituted 5-10 membered heteroaryl, unsubstituted C₃₋₁₂ heteroaralkyl, and unsubstituted 3-10 membered heterocyclyl.

In an embodiment of the first aspect, two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring.

In an embodiment of the first aspect, the PGG is substantially free is less than about 0.5% gallic acid.

In an embodiment of the first aspect, the PGG is substantially free is less than about 0.5% methyl gallate.

In an embodiment of the first aspect, the PGG is applied to at least one of a tendon or a ligament that supports at least one of a kidney, an ureter, a bladder, a urethra, or a sphincter.

In an embodiment of the first aspect, the PGG is placed in a pelvic cavity.

In an embodiment of the first aspect, the PGG is placed in or on a kidney, an ureter, a bladder, a urethra, or a sphincter.

In an embodiment of the first aspect, the PGG is provided in admixture with a poloxamer gel exhibiting a reverse thermosensitive property.

In an embodiment of the first aspect, the poloxamer of the poloxamer gel is poloxamer 407.

In an embodiment of the first aspect, the PGG is provided as a coating or component of an urogynecologic mesh or sling.

In an embodiment of the first aspect, the sling is an autologous sling.

In a second aspect, a method is provided for treating stress urinary incontinence in a patient, comprising: delivering a therapeutic agent to a patient in need thereof, wherein the therapeutic agent comprises pentagalloyl glucose (PGG).

In an embodiment of the second aspect, the therapeutic agent further comprises at least one member of the group consisting of oxybutynin, tolterodine, darifenacin, fesoterodine, solifenacin, trospium, and mirabegron.

In an embodiment of the second aspect, the PGG is applied to at least one of a tendon or a ligament that supports at least one urinary tract organ.

In an embodiment of the second aspect, the PGG is placed in a pelvic cavity.

In an embodiment of the second aspect, the PGG is placed in or on a kidney, an ureter, a bladder, a urethra, or a sphincter.

In an embodiment of the second aspect, the PGG is provided as a coating or component of an urogynecologic mesh or sling.

In an embodiment of the second aspect, the PGG is provided in admixture with a poloxamer gel exhibiting a reverse thermosensitive property.

In an embodiment of the second aspect, the poloxamer of the poloxamer gel is poloxamer 407.

In an embodiment of the second aspect, the PGG is at least 99.9% pure.

In an embodiment of the second aspect, the PGG is substantially free of gallic acid or methyl gallate.

In a third aspect, a composition is provided for treating sudden urinary incontinence in a patient, comprising: a poloxamer gel exhibiting a reverse thermosensitive property.

In an embodiment of the third aspect, the poloxamer gel further comprises pentagalloyl glucose (PGG).

In an embodiment of the third aspect, the poloxamer gel is provided as a coating or component of an urogynecologic mesh or sling.

In an embodiment of the third aspect, the poloxamer of the poloxamer gel is poloxamer 407.

In an embodiment of the third aspect, the PGG is at least 99.9% pure.

In an embodiment of the third aspect, the PGG is substantially free of gallic acid or methyl gallate.

In a fourth aspect, a method is provided for treating sudden urinary incontinence in a patient, comprising: delivering to a patient in need thereof a composition of the third aspect or any of its embodiments.

In an embodiment of the fourth aspect, the poloxamer gel is configured, after delivery to the patient, to support at least one of a kidney, an ureter, a bladder, a urethra, or a sphincter.

In an embodiment of the fourth aspect, the poloxamer gel is configured, after delivery to the patient, to plug the urethra.

In an embodiment of the fourth aspect, the poloxamer gel is configured, after delivery to the patient, to bulk an area around the urethra.

In an embodiment of the fourth aspect, the poloxamer gel is configured to deliver the PGG to a kidney, an ureter, a bladder, a urethra, or a sphincter, a pelvic cavity, or a ligament or tendon supporting a urinary tract organ.

### Treatment of Congestive Heart Failure

Some embodiments provide a composition for treating congestive heart failure comprising a compound of the following Formula:

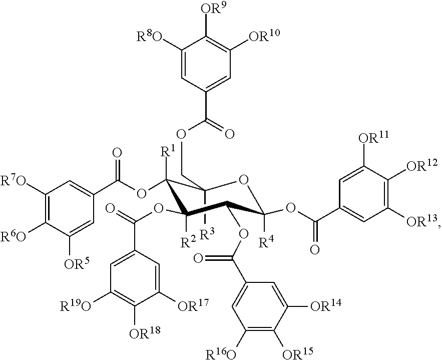

or a pharmaceutically acceptable salt thereof, wherein: R¹-R¹⁹ have any of the values described herein, and wherein the composition is substantially free of gallic acid or methyl gallate. In some embodiments, substantially free is less than about 0.5% gallic acid. In some embodiments, substantially free is less than about 0.5% methyl gallate.

In some embodiments, R¹, R², R³ and R⁴ are each independently hydrogen or R^{A}; R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each independently hydrogen or R^{B}; each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, —NR^{Y}C(═X)OR^{X}, unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl; each R^{B} is independently selected from the group consisting of —C(═X)R^{Z}, —C(═X)N(R^{Y})₂, —C(═X)OR^{X}, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, or two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring; each X is independently oxygen (O) or sulfur (S); each R^{X} and R^{Y} is independently selected from the group consisting of hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl; and each R^{Z} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl.

In some embodiments, at least one of R¹, R², R³ and R⁴ is R^{A} In some embodiments, at least two of R¹, R², R³ and R⁴ are R^{A}. In some embodiments, each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, and —NR^{Y}C(═X)OR^{X}. In some embodiments, each R^{A} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl. In some embodiments, each R^{A} is independently selected from the group consisting of unsubstituted C₁₋₁₂alkoxy, unsubstituted C₁₋₈alkyl, unsubstituted C_{6 or 10}aryl, unsubstituted C₇₋₁₂aralkyl, unsubstituted 5-10 membered heteroaryl, unsubstituted C₃₋₁₂ heteroaralkyl, and unsubstituted 3-10 membered heterocyclyl. In some embodiments, R¹, R², R³ and R⁴ are each hydrogen. In some embodiments, R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each hydrogen. In some embodiments, at least one of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ is R^{B}. In some embodiments, at least two of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are R^{B}. In some embodiments, at least three of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are R^{B}. In some embodiments, each R^{B} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl. In some embodiments, each R^{B} is independently selected from the group consisting of unsubstituted C₁₋₁₂alkoxy, unsubstituted C₁₋₈alkyl, unsubstituted C_{6 or 10}aryl, unsubstituted C₇₋₁₂aralkyl, unsubstituted 5-10 membered heteroaryl, unsubstituted C₃₋₁₂ heteroaralkyl, and unsubstituted 3-10 membered heterocyclyl. In some embodiments, two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. The pharmaceutical composition may be formulated for oral, topical, intravenous, or intravitreal administration. In some embodiments, the pharmaceutical composition is formulated as a unit dose.

In yet another aspect, the present disclosure provides methods of treating and/or preventing congestive heart failure in a patient in need thereof, comprising administering to the patient a composition described herein in an amount sufficient to treat and/or prevent the disease or disorder. In some embodiments, the present disclosure provides methods of treating congestive heart failure.

Some embodiments provide a kit for treating congestive heart failure, comprising: a compound of Formula (I) having a purity greater than or equal to 99%; and a hydrolyzer. In some embodiments, the hydrolyzer is ethanol. In some embodiments, the hydrolyzer is dimethyl sulfoxide (DMSO). In some embodiments, the hydrolyzer is contrast media. In some embodiments, the kit further comprises a saline solution.

### Mitral Valve Repair

What is needed in the art are treatment protocols and compositions for stabilization of the organs and tissues affected by degenerative conditions such as mitral valve disease.

In a first aspect, a device for mitral valve implantation or replacement is provided, comprising: an implantable or replacement valve; and pentagalloyl glucose (PGG) having a purity greater than or equal to 99%.

In an embodiment of the first aspect, at least a portion of the implantable or replacement valve is coated with the PGG.

In an embodiment of the first aspect, at least a portion of a component of the implantable or replacement valve is impregnated with the PGG.

In a second aspect, a device for treating a mitral valve disease is provided, comprising: a shaft; and a first balloon attached to a first end of the shaft and comprising a plurality of pores for delivering a therapeutic agent to a mitral valve, an implantation site, or a surgical site.

In an embodiment of the second aspect, the device further comprises an implantable or replacement valve supported by the first balloon.

In an embodiment of the second aspect, the device further comprises a second balloon positioned within the first balloon for expanding the first balloon, the second balloon expandable with saline.

In a third aspect, a kit for treating a mitral valve disease is provided, comprising: the device of the second aspect or any of its embodiments; pentagalloyl glucose (PGG) having a purity greater than or equal to 99%; and a hydrolyzer.

In an embodiment of the third aspect, the hydrolyzer is ethanol.

In an embodiment of the third aspect, the hydrolyzer is dimethyl sulfoxide (DMSO) or contrast media.

In an embodiment of the third aspect, the kit further comprises a saline solution.

In a fourth aspect, a catheter for treating a mitral valve disease is provided, the catheter comprising: an elongate body configured to be introduced into a surgical site of a removed native mitral valve, the elongate body having a proximal end, a distal end, and a main shaft having a lumen extending therethrough; and a first inflatable balloon coupled to the di stal end of the elongate body, the first inflatable balloon having an interior volume in fluid communication with a first inflation lumen, wherein the first inflatable balloon circumferentially surrounds the elongate body, wherein the first inflatable balloon comprises a plurality of pores disposed on a surface of the first inflatable balloon configured to place the interior volume of the first inflatable balloon in fluid communication with a mitral valve, an implantation site, or a surgical site.

In an embodiment of the fourth aspect, the first inflatable balloon is further configured to support an implantable or replacement valve.

In an embodiment of the fourth aspect, the first inflatable balloon is generally toroidal forming an annular interior volume that surrounds the elongate body.

In an embodiment of the fourth aspect, the pores are disposed on a central portion of the first inflatable balloon.

In an embodiment of the fourth aspect, the pores are disposed on a distal portion of the first inflatable balloon.

In an embodiment of the fourth aspect, the pores are not disposed on a proximal portion of the first inflatable balloon.

In an embodiment of the fourth aspect, the pores are not disposed on any portion of the first inflatable balloon proximal to a maximum expanded diameter of the balloon in an inflated configuration.

In an embodiment of the fourth aspect, the maximum expanded diameter of the first inflatable balloon is greater than the maximum expanded diameter of the first inflatable balloon.

In an embodiment of the fourth aspect, the catheter further comprises a second inflatable balloon disposed within the interior volume of the first inflatable balloon, the second inflatable balloon having an interior volume in fluid communication with a second inflation lumen.

In an embodiment of the fourth aspect, expansion of the second inflatable balloon is configured to at least partially expand the first inflatable balloon.

In an embodiment of the fourth aspect, expansion of the second inflatable balloon is configured to facilitate expulsion of at least a partial volume of inflation fluid disposed within the interior volume of the first inflatable balloon through the pores into the environment of the mitral valve, the implantation site, or the surgical site.

In a fifth aspect, a kit for treating a mitral valve disease is provided, comprising: the catheter of any one of Claims 11 to 21; pentagalloyl glucose (PGG) having a purity greater than or equal to 99%; and a hydrolyzer.

In an embodiment of the fifth aspect, the hydrolyzer is ethanol.

In an embodiment of the fifth aspect, the hydrolyzer is dimethyl sulfoxide (DMSO) or contrast media.

In an embodiment of the fifth aspect, the kit further comprises a saline solution.

In a sixth aspect, a method for treating a mitral valve disease of a patient is provided comprising: positioning a first balloon in a mitral valve; expanding the first balloon such that it forces the mitral valve into an open position, with surfaces of the mitral valve in contact with a surface of the first balloon; and delivering a therapeutic agent to the mitral valve through pores in the first balloon.

In a seventh aspect, a method for treating a mitral valve disease of a patient is provided, comprising: positioning a first balloon adjacent a surgical site of a removed native mitral valve, the first balloon supporting a replacement valve; expanding the first balloon to expand the replacement valve; and delivering a therapeutic agent to the surgical site through pores in the first balloon.

In an eighth aspect, a method for treating a mitral valve disease of a patient is provided, comprising: positioning a first balloon in a mitral valve, the first balloon supporting an implantable valve; expanding the first balloon such that the implantable valve is implanted in the mitral valve; and delivering a therapeutic agent to the implantation site through pores in the first balloon.

In an embodiment of the sixth, seventh, or eighth aspect, expanding the first balloon comprises introducing an inflation fluid into an interior volume of the first balloon.

In an embodiment of the sixth, seventh, or eighth aspect, delivering the therapeutic agent comprises introducing a solution comprising the therapeutic agent into an interior volume of the first balloon, the introduction of the solution being configured to expand and/or maintain an expanded state of the first balloon.

In an embodiment of the sixth, seventh, or eighth aspect, expanding the first balloon comprises maintaining a pressure within an interior volume of the second balloon greater than a diastolic blood pressure of the patient and less than a systolic blood pressure of the patient.

In an embodiment of the sixth, seventh, or eighth aspect, expanding the first balloon and delivering the therapeutic agent through the pores comprises introducing a solution into an interior volume of the first balloon, and wherein the solution is introduced at a first volumetric flow rate to expand the first balloon and the solution is introduced at a second volumetric flow rate to deliver the therapeutic agent through the pores, the first volumetric flow rate being greater than or equal to the second volumetric flow rate.

In an embodiment of the sixth, seventh, or eighth aspect, the first volumetric flow rate is greater than the second volumetric flow rate.

In an embodiment of the sixth, seventh, or eighth aspect, blood flow is occluded by the first balloon no longer than approximately 3 minutes.

In an embodiment of the sixth, seventh, or eighth aspect, at least 1 mL of solution comprising the therapeutic agent is delivered while downstream blood flow and retrograde blood flow is occluded.

In an embodiment of the sixth, seventh, or eighth aspect, expanding the first balloon comprises inflating a second balloon disposed within an interior volume of the first balloon.

In an embodiment of the sixth, seventh, or eighth aspect, delivering the therapeutic agent comprises inflating a second balloon disposed within an interior volume of the first balloon to force a volume of solution comprising the therapeutic agent within the interior volume of the first balloon through the pores.

In an embodiment of the sixth, seventh, or eighth aspect, the therapeutic agent comprises pentagalloyl glucose (PGG).

In an embodiment of the sixth, seventh, or eighth aspect, the PGG is at least 99.9% pure.

In an embodiment of the sixth, seventh, or eighth aspect, the therapeutic agent is substantially free of gallic acid or methyl gallate.

In a ninth aspect, a method for treating a mitral valve disease of a patient is provided, comprising: delivering a therapeutic agent to a mitral valve, wherein the therapeutic agent comprises pentagalloyl glucose (PGG).

In an embodiment of the ninth aspect, the PGG is at least 99.9% pure.

In an embodiment of the ninth aspect, the therapeutic agent is substantially free of gallic acid or methyl gallate.

### Transcatheter Aortic Valve Replacement

What is needed in the art are treatment protocols and compositions for stabilization of the organs and tissues affected by degenerative conditions such as aortic valve stenosis, e.g., by transcatheter aortic valve replacement.

In a first aspect, a device for transcatheter aortic valve replacement (TAVR) is provided, comprising: a replacement valve; and pentagalloyl glucose (PGG) having a purity greater than or equal to 99%.

In an embodiment of the first aspect, at least a portion of the replacement valve is coated with the PGG.

In an embodiment of the first aspect, at least a portion of a component of the replacement valve is impregnated with the PGG.

In a second aspect, a device for treating an aortic valve stenosis is provided, comprising: a shaft; a first balloon attached to a first end of the shaft and comprising a plurality of pores for delivering a therapeutic agent to a surgical site; and a replacement valve supported by the first balloon.

In an embodiment of the second aspect, the device further comprises a second balloon positioned within the first balloon for expanding the first balloon, the second balloon expandable with saline.

In a third aspect, a kit for treating aortic valve stenosis is provided, comprising: the device of the second aspect or any of its embodiments; pentagalloyl glucose (PGG) having a purity greater than or equal to 99%; and a hydrolyzer.

In an embodiment of the third aspect, the hydrolyzer is ethanol.

In an embodiment of the third aspect, the hydrolyzer is dimethyl sulfoxide (DMSO) or contrast media.

In an embodiment of the third aspect, the kit further comprises a saline solution.

In a fourth aspect, a catheter for treating an aortic valve stenosis is provided, the catheter comprising: an elongate body configured to be introduced into a surgical site of a removed native aortic valve, the elongate body having a proximal end, a distal end, and a main shaft having a lumen extending therethrough; and a first inflatable balloon coupled to the distal end of the elongate body, the first inflatable balloon having an interior volume in fluid communication with a first inflation lumen, wherein the first inflatable balloon circumferentially surrounds the elongate body, wherein the first inflatable balloon is configured to support a replacement valve, and wherein the first inflatable balloon comprises a plurality of pores disposed on a surface of the first inflatable balloon configured to place the interior volume of the first inflatable balloon in fluid communication with a surgical site.

In an embodiment of the fourth aspect, the first inflatable balloon is generally toroidal forming an annular interior volume that surrounds the elongate body.

In an embodiment of the fourth aspect, the pores are disposed on a central portion of the first inflatable balloon.

In an embodiment of the fourth aspect, the pores are disposed on a distal portion of the first inflatable balloon.

In an embodiment of the fourth aspect, the pores are not disposed on a proximal portion of the first inflatable balloon.

In an embodiment of the fourth aspect, the pores are not disposed on any portion of the first inflatable balloon proximal to a maximum expanded diameter of the balloon in an inflated configuration.

In an embodiment of the fourth aspect, the maximum expanded diameter of the first inflatable balloon is greater than the maximum expanded diameter of the first inflatable balloon.

In an embodiment of the fourth aspect, the catheter further comprises a second inflatable balloon disposed within the interior volume of the first inflatable balloon, the second inflatable balloon having an interior volume in fluid communication with a second inflation lumen.

In an embodiment of the fourth aspect, expansion of the second inflatable balloon is configured to at least partially expand the first inflatable balloon.

In an embodiment of the fourth aspect, expansion of the second inflatable balloon is configured to facilitate expulsion of at least a partial volume of inflation fluid disposed within the interior volume of the first inflatable balloon through the pores into the environment of the surgical site.

In a fifth aspect, a kit for treating aortic valve stenosis is provided, comprising: the catheter of the fourth aspect or any of its embodiments; pentagalloyl glucose (PGG) having a purity greater than or equal to 99%; and a hydrolyzer.

In an embodiment of the fifth aspect, the hydrolyzer is ethanol.

In an embodiment of the fifth aspect, the hydrolyzer is dimethyl sulfoxide (DMSO) or contrast media.

In an embodiment of the fifth aspect, the kit further comprises a saline solution.

In a sixth aspect, a method for treating an aortic valve stenosis of a patient is provided, comprising: positioning a first balloon adjacent a surgical site of a removed native aortic valve, the first balloon supporting a replacement valve; expanding the first balloon to expand the replacement valve; and delivering a therapeutic agent to the surgical site through pores in the first balloon.

In an embodiment of the sixth aspect, expanding the first balloon comprises introducing an inflation fluid into an interior volume of the first balloon.

In an embodiment of the sixth aspect, delivering the therapeutic agent comprises introducing a solution comprising the therapeutic agent into an interior volume of the first balloon, the introduction of the solution being configured to expand and/or maintain an expanded state of the first balloon.

In an embodiment of the sixth aspect, wherein expanding the first balloon comprises maintaining a pressure within an interior volume of the first balloon greater than a diastolic blood pressure of the patient and less than a systolic blood pressure of the patient.

In an embodiment of the sixth aspect, expanding the first balloon and delivering the therapeutic agent through the pores comprises introducing a solution into an interior volume of the first balloon, and wherein the solution is introduced at a first volumetric flow rate to expand the first balloon and the solution is introduced at a second volumetric flow rate to deliver the therapeutic agent through the pores, the first volumetric flow rate being greater than or equal to the second volumetric flow rate.

In an embodiment of the sixth aspect, the first volumetric flow rate is greater than the second volumetric flow rate.

In an embodiment of the sixth aspect, blood flow is occluded within the surgical site for no longer than approximately 3 minutes.

In an embodiment of the sixth aspect, at least 1 mL of solution comprising the therapeutic agent is delivered while downstream blood flow and retrograde blood flow through the surgical site is occluded.

In an embodiment of the sixth aspect, expanding the first balloon comprises inflating a second balloon disposed within an interior volume of the first balloon.

In an embodiment of the sixth aspect, delivering the therapeutic agent comprises inflating a second balloon disposed within an interior volume of the first balloon to force a volume of solution comprising the therapeutic agent within the interior volume of the first balloon through the pores.

In an embodiment of the sixth aspect, the therapeutic agent comprises pentagalloyl glucose (PGG).

In an embodiment of the sixth aspect, the PGG is at least 99.9% pure.

In an embodiment of the sixth aspect, the therapeutic agent is substantially free of gallic acid or methyl gallate.

### Transcatheter Aortic Valve Implantation

What is needed in the art are treatment protocols and compositions for stabilization of the organs and tissues affected by degenerative conditions such as aortic valve stenosis, e.g., by transcatheter aortic valve implantation.

In a first aspect, a device is provided for transcatheter aortic valve implantation, comprising: an implantable valve; and PGG having a purity greater than or equal to 99%.

In an embodiment of the first aspect, at least a portion of the implantable valve is coated with the PGG.

In an embodiment of the first aspect, at least a portion of a component of the implantable valve is impregnated with the PGG.

In a second aspect, a device is provided for treating an aortic valve stenosis, comprising: a shaft; a first balloon attached to a first end of the shaft; a second balloon attached to a second end of the shaft; and a transcatheter implantable valve supported by the second balloon, the second balloon comprising a plurality of pores for delivering a therapeutic agent to an implantation site.

In an embodiment of the second aspect, the first balloon is positioned near a distal end of the shaft for anchoring the device and stopping downstream blood flow, and wherein the second balloon is positioned near a proximal end of the shaft for stopping retrograde blood flow.

In an embodiment of the second aspect, the second balloon is positioned near a distal end of the shaft for anchoring the device and stopping downstream blood flow, and wherein the first balloon is positioned near a proximal end of the shaft for stopping retrograde blood flow.

In an embodiment of the second aspect, the device further comprises a third balloon positioned within the second balloon for expanding the second balloon, the third balloon expandable with saline.

In a third aspect, a kit is provided for treating aortic valve stenosis, comprising: the device of the second aspect or any of its embodiments; PGG having a purity greater than or equal to 99%; and a hydrolyzer.

In an embodiment of the third aspect, the hydrolyzer is ethanol.

In an embodiment of the third aspect, the hydrolyzer is dimethyl sulfoxide (DMSO) or contrast media.

In an embodiment of the third aspect, the kit further comprises a saline solution.

In a fourth aspect, a catheter is provided for treating an aortic valve stenosis, the catheter comprising: an elongate body configured to be introduced into an aortic valve, the elongate body having a proximal end, a distal end, and a main shaft having a lumen extending therethrough; a first inflatable balloon coupled to the distal end of the elongate body, the first inflatable balloon having an interior volume in fluid communication with a first inflation lumen; and a second inflatable balloon coupled to the elongate body proximally to the first inflatable balloon, the second inflatable balloon having an interior volume in fluid communication with a second inflation lumen, wherein the second inflatable balloon circumferentially surrounds the elongate body, and wherein the second inflatable balloon comprises a plurality of pores disposed on a surface of the second inflatable balloon configured to place the interior volume of the second inflatable balloon in fluid communication with an intravascular environment of the aortic valve.

In an embodiment of the fourth aspect, the main shaft extends through the second inflatable balloon and the distal end of the main shaft forms the distal end of the elongate body.

In an embodiment of the fourth aspect, the first inflation lumen and the second inflation lumen are formed within the main shaft.

In an embodiment of the fourth aspect, the elongate body further comprises a second shaft having a lumen extending therethrough, the second shaft being disposed within the lumen of the main shaft, the first inflatable balloon being coupled to a distal end of the second shaft and the second inflatable balloon being coupled to a distal end of the main shaft.

In an embodiment of the fourth aspect, the lumen of the main shaft is the second inflation lumen.

In an embodiment of the fourth aspect, the lumen of the second shaft is the first inflation lumen.

In an embodiment of the fourth aspect, the elongate body extends through the interior volume of the second inflatable balloon.

In an embodiment of the fourth aspect, the second inflatable balloon is generally toroidal forming an annular interior volume that surrounds the elongate body.

In an embodiment of the fourth aspect, the elongate body comprises an intermediate shaft segment positioned between a proximal end of the first inflatable balloon and a distal end of the second inflatable balloon.

In an embodiment of the fourth aspect, the intermediate shaft segment comprises the main shaft.

In an embodiment of the fourth aspect, the intermediate shaft segment comprises the second shaft.

In an embodiment of the fourth aspect, a separation distance between the first inflatable balloon and the second inflatable balloon is fixed.

In an embodiment of the fourth aspect, a separation distance between the first inflatable balloon and the second inflatable balloon is adjustable.

In an embodiment of the fourth aspect, the catheter further comprises a lumen configured to be placed in fluid communication with a volume of the intravascular environment between the first inflatable balloon and the second inflatable balloon.

In an embodiment of the fourth aspect, the pores are disposed on a central portion of the second inflatable balloon.

In an embodiment of the fourth aspect, the pores are disposed on a distal portion of the second inflatable balloon.

In an embodiment of the fourth aspect, the pores are not disposed on a proximal portion of the second inflatable balloon.

In an embodiment of the fourth aspect, the pores are not disposed on any portion of the second inflatable balloon proximal to a maximum expanded diameter of the balloon in an inflated configuration.

In an embodiment of the fourth aspect, the maximum expanded diameter of the second inflatable balloon is greater than the maximum expanded diameter of the first inflatable balloon.

In an embodiment of the fourth aspect, the length of the expanded second inflatable balloon is greater than the length of the expanded first inflatable balloon.

In an embodiment of the fourth aspect, the catheter further comprises a third inflatable balloon disposed within the interior volume of the second inflatable balloon, the third inflatable balloon having an interior volume in fluid communication with a third inflation lumen.

In an embodiment of the fourth aspect, expansion of the third inflatable balloon is configured to at least partially expand the second inflatable balloon.

In an embodiment of the fourth aspect, expansion of the third inflatable balloon is configured to facilitate expulsion of at least a partial volume of inflation fluid disposed within the interior volume of the second inflatable balloon through the pores into the intravascular environment.

In a fifth aspect, a kit is provided for treating aortic valve stenosis, comprising: the catheter of the fourth aspect or any of its embodiments; PGG having a purity greater than or equal to 99%; and a hydrolyzer.

In an embodiment of the fifth aspect, the hydrolyzer is ethanol.

In an embodiment of the fifth aspect, the hydrolyzer is dimethyl sulfoxide (DMSO) or contrast media.

In an embodiment of the fifth aspect, the kit further comprises a saline solution.

In a sixth aspect, a method is provided for treating an aortic valve stenosis of a patient, comprising: positioning a first balloon upstream the aortic valve; positioning a second balloon adjacent the aortic valve at an implantation site, the second balloon supporting a transcatheter implantable valve; inflating the first balloon to occlude downstream blood flow; expanding the second balloon to occlude retrograde blood flow and/or expand the implantable valve; and delivering a therapeutic agent to the implantation site through pores in the second balloon.

In an embodiment of the sixth aspect, expanding the second balloon comprises introducing an inflation fluid into an interior volume of the second balloon.

In an embodiment of the sixth aspect, delivering the therapeutic agent comprises introducing a solution comprising the therapeutic agent into an interior volume of the second balloon, the introduction of the solution being configured to expand and/or maintain an expanded state of the second balloon.

In an embodiment of the sixth aspect, inflating the first balloon and expanding the second balloon creates a sealed volume within the implantation site between the first balloon and the second balloon.

In an embodiment of the sixth aspect, delivering the therapeutic agent comprises introducing the therapeutic agent into the sealed volume.

In an embodiment of the sixth aspect, the therapeutic agent is not delivered outside of the sealed volume while the sealed volume is established.

In an embodiment of the sixth aspect, inflating the first balloon anchors the first balloon and the second balloon within the implantation site.

In an embodiment of the sixth aspect, positioning the second balloon in the aortic valve comprises positioning the second balloon across the aortic valve and wherein expanding the second balloon creates a sealed space between the second balloon and the aortic valve.

In an embodiment of the sixth aspect, inflating the first balloon occurs prior to expanding the second balloon.

In an embodiment of the sixth aspect, expanding the second balloon and/or maintaining the second balloon in an expanded state comprises maintaining a pressure within an interior volume of the second balloon greater than a diastolic blood pressure of the patient and less than a systolic blood pressure of the patient.

In an embodiment of the sixth aspect, expanding the second balloon and delivering the therapeutic agent through the pores comprises introducing a solution into an interior volume of the second balloon, and wherein the solution is introduced at a first volumetric flow rate to expand the second balloon and the solution is introduced at a second volumetric flow rate to deliver the therapeutic agent through the pores, the first volumetric flow rate being greater than or equal to the second volumetric flow rate.

In an embodiment of the sixth aspect, the first volumetric flow rate is greater than the second volumetric flow rate.

In an embodiment of the sixth aspect, blood flow is occluded within the aortic valve for no longer than approximately 3 minutes.

In an embodiment of the sixth aspect, at least 1 mL of solution comprising the therapeutic agent is delivered while downstream blood flow and retrograde blood flow through the aortic valve.

In an embodiment of the sixth aspect, expanding the second balloon comprises inflating a third balloon disposed within an interior volume of the second balloon.

In an embodiment of the sixth aspect, delivering the therapeutic agent comprises inflating a third balloon disposed within an interior volume of the second balloon to force a volume of solution comprising the therapeutic agent within the interior volume of the second balloon through the pores.

In an embodiment of the sixth aspect, the therapeutic agent comprises pentagalloyl glucose (PGG).

In an embodiment of the sixth aspect, the PGG is at least 99.9% pure.

In an embodiment of the sixth aspect, the therapeutic agent is substantially free of gallic acid or methyl gallate.

### Treatment of Tumors

What is needed in the art are treatment protocols and compositions for stabilization of the organs and tissues affected by tumors. In particular, treatment protocols utilizing phenolic compounds could provide a safe, less invasive route for the stabilization of the structural architecture in order to attain better outcomes for patients treated for tumors.

Some embodiments provide a composition for treating tumors comprising a compound of the following Formula:

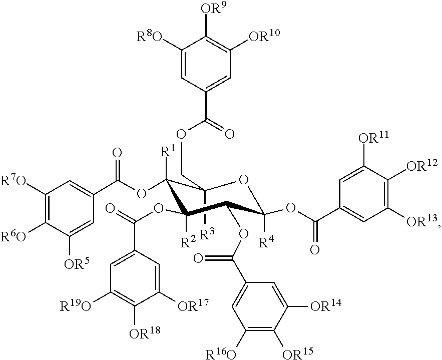

or a pharmaceutically acceptable salt thereof, wherein. R¹-R¹⁹ have any of the values described herein, and wherein the composition is substantially free of gallic acid or methyl gallate. In some embodiments, substantially free is less than about 0.5% gallic acid. In some embodiments, substantially free is less than about 0.5% methyl gallate.

In some embodiments, R¹, R², R³ and R⁴ are each independently hydrogen or R^{A}; R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each independently hydrogen or R^{B}; each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, —NR^{Y}C(═X)OR^{X}, unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl; each R^{B} is independently selected from the group consisting of —C(═X)R^{Z}, —C(═X)N(R^{Y})₂, —C(═X)OR^{X}, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, or two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring; each X is independently oxygen (O) or sulfur (S); each R^{X} and R^{Y} is independently selected from the group consisting of hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl; and each R^{Z} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl.

In some embodiments, at least one of R¹, R², R³ and R⁴ is R^{A} In some embodiments, at least two of R¹, R², R³ and R⁴ are R^{A}. In some embodiments, each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, and —NR^{Y}C(═X)OR^{X}. In some embodiments, each R^{A} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl. In some embodiments, each R^{A} is independently selected from the group consisting of unsubstituted C₁₋₁₂alkoxy, unsubstituted C₁₋₈alkyl, unsubstituted C_{6 or 10}aryl, unsubstituted C₇₋₁₂aralkyl, unsubstituted 5-10 membered heteroaryl, unsubstituted C₃₋₁₂ heteroaralkyl, and unsubstituted 3-10 membered heterocyclyl. In some embodiments, R¹, R², R³ and R⁴ are each hydrogen. In some embodiments, R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each hydrogen. In some embodiments, at least one of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ is R^{B}. In some embodiments, at least two of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are R^{B}. In some embodiments, at least three of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are R^{B}. In some embodiments, each R^{B} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl. In some embodiments, each R^{B} is independently selected from the group consisting of unsubstituted C₁₋₁₂alkoxy, unsubstituted C₁₋₈alkyl, unsubstituted C_{6 or 10}aryl, unsubstituted C₇₋₁₂aralkyl, unsubstituted 5-10 membered heteroaryl, unsubstituted C₃₋₁₂ heteroaralkyl, and unsubstituted 3-10 membered heterocyclyl. In some embodiments, two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. The pharmaceutical composition may be formulated for oral, topical, intravenous, or intravitreal administration. In some embodiments, the pharmaceutical composition is formulated as a unit dose.

In yet another aspect, the present disclosure provides methods of treating and/or preventing tumors in a patient in need thereof, comprising administering to the patient a composition described herein in an amount sufficient to treat and/or prevent the disease or disorder. In some embodiments, the present disclosure provides methods of treating tumors, such as liver or prostate tumors, or the regions adjacent to such tumors so as to improve patient outcomes for treatment of tumors. In certain embodiments, administering the compound reduces the tumor volume, e.g., by 10%, 20%, 30%, 40%, 50%, 60%, 70%, 80%, 90%, or more. In certain embodiments, administering the compound eliminates the tumor. In certain embodiments, administering the compound slows the speed of tumor growth by 10%, 20%, 30%, 40%, 50%, 60%, 70%, 80%, 90%, or more. In certain embodiments, administering the compound halts tumor growth.

Some embodiments provide a method of purifying a compound of Formula (I) comprising: washing a mixture with a solvent to remove substantially all gallic acid or methyl gallate. In some embodiments, the solvent is diethyl ether. In some embodiments, the solvent is selected from the group consisting of methanol, toluene, isopropyl ether, dichloromethane, methyl tert-butyl ether, 2-butanone, and ethyl acetate. In some embodiments, the washing results in a purity of the compound of Formula (I) thereof greater than or equal to 99.10%, 99.20%, 99.30%, 99.40%, 99.50%, 99.60%, 99.70%, 99.80%, 99.90%, 99.91%, 99.92%, 99.93%, 99.4%, 99.95%, 99.96%, 99.97%, 99.98%, or 99.99%.

Some embodiments provide a kit for treating tumors, comprising: a compound of Formula (I) having a purity greater than or equal to 99%; and a hydrolyzer. In some embodiments, the hydrolyzer is ethanol. In some embodiments, the hydrolyzer is dimethyl sulfoxide (DMSO). In some embodiments, the hydrolyzer is contrast media. In some embodiments, the kit further comprises a saline solution.

Tumors amenable to treatment by the methods disclosed herein include liver and prostate tumors. In other embodiments, other solid tumors can be treated. The solid tumor may be a primary or a metastatic tumor. Exemplary solid tumors include tumors of the breast, lung especially non-small cell lung cancer, colon, stomach, liver, kidney, brain, head and neck especially squamous cell carcinoma of the head and neck, thyroid, ovary, testes, liver, melanoma, prostate especially androgen-independent (hormone refractory) prostate cancer, neuroblastoma and gastric adenocarcinoma including adenocarcinoma of the gastrooesophageal junction.

In some embodiments, the cancer, is selected from the group consisting of breast cancer, ovarian cancer (e.g., recurrent ovarian cancer), testicular cancer (e.g., cis-platin-resistant germ cell cancer), prostate cancer (e.g., bone metastatic prostate cancer, prostatic neoplasms, hormone-refractory prostate cancer, castration resistant prostate cancer, advanced prostate cancer), liver cancer, dedifferentiated liposarcoma, urothelial carcinoma of the urinary bladder (e.g., urothelium transitional cell carcinoma (TCCU)), adrenocortical carcinoma, brain cancer (e.g., recurrent malignant glioma), AIL (acute myeloid leukemia) and CLL (chronic lymphocytic leukemia). In some embodiments, the cancer is prostate cancer or breast cancer. In some embodiments the cancer is prostate cancer, for example hormone-refractory prostate cancer, or for example metastatic castration-resistant prostate cancer (mCRPC). In some embodiments the cancer is breast cancer.

### Treatment of Pelvic Organ Prolapse

In a first aspect, a method is provided of treating pelvic organ prolapse, comprising: administering, to a patient in need thereof, a composition comprising a compound of Formula:

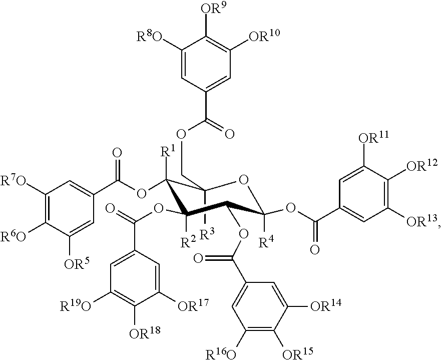

or a pharmaceutically acceptable salt thereof, wherein: R¹, R², R³ and R⁴ are each independently hydrogen or R^{A}; R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each independently hydrogen or R^{B}; each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, —NR^{Y}C(═X)OR^{X}, unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl; each R^{B} is independently selected from the group consisting of —C(═X)R^{Z}, —C(═X)N(R^{Y})₂, —C(═X)OR^{X}, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, or two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring; each X is independently oxygen (O) or sulfur (S); each R^{X} and R^{Y} is independently selected from the group consisting of hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl; and each R^{Z} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, and wherein the composition is substantially free of gallic acid or methyl gallate.

In an embodiment of the first aspect, at least one of R¹, R², R³ and R⁴ is R^{A}.

In an embodiment of the first aspect, at least two of R¹, R², R³ and R⁴ are R^{A}.

In an embodiment of the first aspect, each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, and —NR^{Y}C(═X)OR^{X}.

In an embodiment of the first aspect, each R^{A} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl.

In an embodiment of the first aspect, each R^{A} is independently selected from the group consisting of unsubstituted C₁₋₁₂alkoxy, unsubstituted C₁₋₈alkyl, unsubstituted C_{6 or 10}aryl, unsubstituted C₇₋₁₂aralkyl, unsubstituted 5-10 membered heteroaryl, unsubstituted C₃₋₁₂ heteroaralkyl, and unsubstituted 3-10 membered heterocyclyl.

In an embodiment of the first aspect, R¹, R², R³ and R⁴ are each hydrogen.

In an embodiment of the first aspect, R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each hydrogen.

In an embodiment of the first aspect, at least one of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ is R^{B}.

In an embodiment of the first aspect, at least two of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are R^{B}.

In an embodiment of the first aspect, at least three of R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are R^{B}.

In an embodiment of the first aspect, each R^{B} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl.

In an embodiment of the first aspect, each R^{B} is independently selected from the group consisting of unsubstituted C₁₋₁₂alkoxy, unsubstituted C₁₋₈alkyl, unsubstituted C_{6 or 10}aryl, unsubstituted C₇₋₁₂aralkyl, unsubstituted 5-10 membered heteroaryl, unsubstituted C₃₋₁₂ heteroaralkyl, and unsubstituted 3-10 membered heterocyclyl.

In an embodiment of the first aspect, two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring.

In an embodiment of the first aspect, the PGG is substantially free is less than about 0.5% gallic acid.

In an embodiment of the first aspect, the PGG is substantially free is less than about 0.5% methyl gallate.

In an embodiment of the first aspect, the PGG is applied to at least one of a tendon or a ligament that supports at least one pelvic organ.

In an embodiment of the first aspect, the PGG is placed in a pelvic cavity.

In an embodiment of the first aspect, the PGG is placed in or on a pelvic organ.

In an embodiment of the first aspect, the PGG is provided as a coating or component of an urogynecologic mesh.

In an embodiment of the first aspect, the PGG is provided in admixture with a poloxamer gel exhibiting a reverse thermosensitive property.

In an embodiment of the first aspect, the poloxamer of the poloxamer gel is poloxamer 407.

In a second aspect, a method is provided for treating pelvic organ prolapse in a patient, comprising: delivering a therapeutic agent to a patient in need thereof, wherein the therapeutic agent comprises pentagalloyl glucose (PGG).

In an embodiment of the second aspect, the PGG is applied to at least one of a tendon or a ligament that supports at least one pelvic organ.

In an embodiment of the second aspect, the PGG is placed in a pelvic cavity.

In an embodiment of the second aspect, the PGG is placed in or on a pelvic organ.

In an embodiment of the second aspect, the PGG is provided as a coating or component of an urogynecologic mesh.

In an embodiment of the second aspect, the PGG is provided in admixture with a poloxamer gel exhibiting a reverse thermosensitive property.

In an embodiment of the second aspect, the poloxamer of the poloxamer gel is poloxamer 407.

In an embodiment of the second aspect, the PGG is at least 99.9% pure.

In an embodiment of the second aspect, the PGG is substantially free of gallic acid or methyl gallate.

In a third aspect, a composition is provided for treating pelvic organ prolapse in a patient, comprising: a poloxamer gel exhibiting a reverse thermosensitive property.

In an embodiment of the third aspect, the poloxamer gel further comprises pentagalloyl glucose (PGG).

In an embodiment of the third aspect, the poloxamer gel is provided as a coating or component of an urogynecologic mesh.

In an embodiment of the third aspect, the poloxamer of the poloxamer gel is poloxamer 407.

In an embodiment of the third aspect, the PGG is at least 99.9% pure.

In an embodiment of the third aspect, the PGG is substantially free of gallic acid or methyl gallate.

In a fourth aspect, a method is provided for treating pelvic organ prolapse in a patient, comprising: delivering to a patient in need thereof a composition of the third aspect or any of its embodiments.

In an embodiment of the fourth aspect, the poloxamer gel is configured, after delivery to the patient, to support at least one pelvic organ.

In an embodiment of the fourth aspect, the poloxamer gel is configured to deliver the PGG to a pelvic tissue, a pelvic cavity, a pelvic organ, or a ligament or tendon supporting a pelvic organ.

Any feature of any aspect or embodiment is independently combinable, in whole or in part, with one or more other features or aspects as described herein. Any feature of any aspect or embodiment may be made optional to the aspect or embodiment.

### BRIEF DESCRIPTION OF THE DRAWINGS

The features and advantages of the systems, devices, and methods described herein will become apparent from the following description, taken in conjunction with the accompanying drawings. These drawings depict only several embodiments in accordance with the disclosure and are not to be considered limiting of its scope. In the drawings, similar reference numbers or symbols typically identify similar components, unless context dictates otherwise. The drawings may not be drawn to scale.

FIG. 1A depicts the chemical structure of 1,2,3,4,6-pentagalloyl glucose (PGG) in a preferred embodiment.

FIG. 1B depicts the chemical structure of gallic acid, a common toxic impurity in the production of PGG.

FIG. 1C depicts the chemical structure of methyl gallate, a common toxic impurity in the production of PGG.

FIGS. 2A-2B schematically depict various examples of a delivery catheter for the delivery of PGG or another therapeutic agent to vascular to be subjected to angioplasty or to a surgical site. FIG. 2A depicts a delivery catheter in which the balloon is coupled at a proximal end to the distal end of the main shaft. FIG. 2B depicts a delivery catheter in which the balloon is a generally toroidal balloon coupled to the distal end of the main shaft and surrounds the secondary shaft.

FIG. 3 schematically depicts various examples of a balloon of a delivery catheter supporting an implantable stent, an implantable valve, or a replacement valve.

FIGS. 4A-4C schematically depict various examples of a delivery catheter comprising an inner balloon disposed within the weeping balloon. FIG. 4A depicts the inner balloon coupled at a proximal end to the distal end of the main shaft. FIG. 4B depicts the inner balloon coupled at proximal and distal ends to the secondary shaft. FIG. 4C depicts the inner balloon coupled at proximal and distal ends to the main shaft.

### DETAILED DESCRIPTION

### Methods of Treatment

### Tendon Repair

Tendon repair is done to bring back normal movement to a joint. Tendon injury may occur anywhere in the body where there are tendons. The joints most commonly affected by tendon injuries are the shoulders, elbows, ankles, knees, and fingers. Tendon injury may occur from a laceration or cut that goes past the skin and through the tendon. Tendon injury is also common from contact sports injuries such as football, wrestling, and rugby. According to the American Academy of Orthopedic Surgeons, “jersey finger” is one of the most common sports injuries affecting the tendons. It may occur when one player grabs the jersey of another player and gets their finger caught on the jersey. When the other player moves, the finger is pulled, and in turn the tendon is pulled off the bone. Tendon damage can also occur in rheumatoid arthritis, an inflammatory disease of the joints, which causes tendons to tear more easily.

Generally, during tendon repair a surgeon will make one or more small incisions in the skin over the damaged tendon, sew the torn ends of the tendon together, check the surrounding tissue to make sure no other injuries have occurred, such as injury to the blood vessels or nerves, close the incision, cover the area with sterile bandages or dressings, and potentially immobilize or splint the joint so as to allow the tendon to heal. If there is not enough healthy tendon to reconnect, the surgeon may perform a tendon graft using a piece of tendon from another part of the body (such as the foot or toe). On occasion, a tendon transfer (moving a tendon from one area to another) may be useful in restoring function. Anesthesia is used to prevent the patient from feeling pain during the tendon repair surgery.

Tendon repairs can be very successful if accompanied by proper physical therapy or occupational therapy. As a general rule, the sooner tendon repair surgery is done after the injury, the easier the surgery is and the easier the recovery. In some cases, long-term complications may develop. Stiffness may be long-lasting. Some tendon injuries, such as injuries to the flexor tendon in the arm, can be very difficult to repair.

Tendon repair or replacement surgery can result in tendon laxity. Laxity of tendons, or laxity of ligaments is a condition where the tendons or ligaments are “loose”. In both post-surgery and other indications, ligamentous laxity can be a cause of chronic body pain characterized by loose ligaments. When this condition affects joints in the entire body, it is called generalized joint hypermobility, which occurs in about ten percent of the population, and may be genetic. Loose ligaments can appear in a variety of ways and levels of severity. It also does not always affect the entire body. One could have loose ligaments of the feet, but not of the arms. Someone with ligamentous laxity, by definition, has loose ligaments. Unlike other, more pervasive diseases, the diagnosis does not require the presence of loose tendons, muscles or blood vessels, hyperlax skin or other connective tissue problems. In heritable connective tissue disorders associated with joint hyper-mobility (such as Marfan syndrome and Ehlers-Danlos syndrome types I-III, VII, and XI), the joint laxity usually is apparent before adulthood. However, age of onset and extent of joint laxity are variable in Marfan syndrome, and joint laxity may be confined to the hands alone, as in Ehlers-Danlos syndrome type I. In addition, ligamentous laxity may appear in conjunction with physical co-ordination conditions such as Dyspraxia. While ligamentous laxity may be genetic and affect an individual from a very early age, it can also be the result of an injury. Injuries, especially those involving the joints, invariably damage ligaments either by stretching them abnormally or even tearing them.

Loose or lax ligaments in turn are not capable of supporting joints as effectively as healthy ones, making the affected individual prone to further injury as well as compensation for the weakness using other parts of the body. Afflicted individuals may improve over time and lose some of their juvenile hyperlaxity as they age. Individuals over age 40 often have recurrent joint problems and almost always have chronic pain. Back patients with ligamentous laxity in the area of the spine may also experience osteoarthritis and disc degeneration.

In the case of extreme laxity, or hypermobility, affected individuals often have a decreased ability to sense joint position, which can contribute to joint damage. The resulting poor limb positions can lead to the acceleration of degenerative joint conditions. Many hypermobility patients have osteoarthritis, disorders involving nerve compression, chondromalacia patellae, excessive anterior mandibular movement, mitral valve prolapse, uterine prolapse and varicose veins.

Arthralgia, or symptoms such as frequent sprained ankles, shoulder dislocations, knee effusions and back problems are common among individuals with ligamentous laxity. Afflicted individuals are also prone to bone dislocation, and those with a sedentary job often report back pain. In addition, people may experience referred pain, that is, pain in an area of the body away from the injured or otherwise affected site.

Individuals with extremely lax, or hypermobile joints, can be identified by their ability to bend their elbows, knees or hips past a position of neutrality. They may also be able to easily touch their hands flat to the floor while bending forward from the waist. The ability to touch the thumb to the forearm is also common.

Referred pain is created by ligamentous laxity around a joint, but is felt at some distance from the injury. (Pain will not only occur at the site of the injury and loose ligaments, but may also be referred to other parts of the body.) These painful points that refer pain elsewhere are called trigger points, and will be dealt with later. Abnormal joint movement also creates many “protective actions” by adjacent tissues. Muscles will contract in spasm in an attempt to pull the joint back to the correct location or stabilize it to protect it from further damage. When this occurs in the back, orthopedic surgeons will often try to reduce vertebral instability by fusing the vertebrae with bone and/or metal fixation.

Those who have loose ligaments in the legs and feet may appear to have flat feet. While their feet have an arch when not supporting weight, when stood upon, the arch will flatten. This is because the loose ligaments cannot support the arch in the way that they should. This can make walking and standing painful and tiring. Pain will usually occur in the feet and lower legs, but can also spread to the back due to abnormal standing and walking habits. Wearing shoes that have good arch support can help minimize the discomfort. The underlying problem, however, is not solved by wearing shoes with arch supports, or worsened by wearing shoes without arch support. There is currently no cure for the condition.

In addition, people with ligamentous laxity often have clumsy or deliberate gaits, owing to the body having to overcompensate for the greater amount of energy required to offset the weakened ligaments. The feet may be spread apart at a wide angle, and the knees may flex backwards slightly after each stride. Those who have this disease may experience sprained ankles more frequently than other people.

In most people, ligaments (which connect bones to each other) are naturally tight in such a way that the joints are restricted to “normal” ranges of motion. This creates normal joint stability. If muscular control does not compensate for ligamentous laxity, joint instability may result. The trait is almost certainly hereditary, and is usually something the affected person would just be aware of, rather than a serious medical condition. However, widespread laxity of other connective tissue may be a sign of Ehlers-Danlos syndrome.

Ligamentous laxity may also result from injury, such as from a vehicle accident. It can result from whiplash and be overlooked for years by doctors who are not looking for it, despite the chronic pain that accompanies the resultant spinal instability. Ligamentous laxity will show up on an upright magnetic resonance imaging (MRI), the only kind of MRI that will show tendon damage. It can be seen in standing stress radiographs in flexion, extension, and neutral views as well, and also digital motion X-ray, or DMX.

An advantage to having lax ligaments and joints is the ability to withstand pain from hyperextension; however, this is also a disadvantage as a lack of perceived pain can prevent a person from removing the ligament from insult, leading to ligament damage. People with hypermobile joints (or “double-jointed” people), almost by definition, have lax ligaments.

### Soft Tissue

Soft tissue is a term that refers to a group of cells working together to connect, envelope, support and/or move the body structures around it. The accurate use of this term spans several tissue types and body systems including muscle, connective, skin (integument system) nervous system and circulatory system (blood vessels), but not generally bone or skeletal system.

Muscles and other soft tissues play important roles the health. For example, many times people complain of back pain when what they really have is tight hip muscles that pull the spine out of alignment. Another soft tissue, fascia, is a covering that surrounds muscles at every level, from the microscopic cell, to the fiber bundles that comprise individual muscles to the muscles themselves, plus muscle groups and the entire musculoskeletal system. The purpose of fascia is to support the integrity and movement of muscles. Ideally, fascial fibers glide smoothly, but they can become “stuck” because of injury. Because the fascia wraps around muscles, when it becomes stuck, it can be mistaken for muscle tension. Ligaments are tough bands of connective tissue that strap the bones of a joint together. Because ligaments cross joints, they help prevent excessive and/or potentially harmful, movement. Tendons, made of a different type of connective tissue, attach muscles to bones. When a muscle contracts, it tugs on the tendon that arises from it, and the tendon moves the bone to which it is attached.

Damage to soft tissue can require surgery to correct. Soft-tissue surgeries are primarily aimed at improving joint stability by repairing the functional length of muscles, tendons, and ligaments. For example, tendon repair is surgery done to treat a torn or otherwise damaged tendon. When tendon damage occurs, movement may be seriously limited and the damaged area may feel weak or painful. Tendon repair surgery may be helpful for people who have tendon injuries that are making it difficult for them to move a joint or are very painful. Tendon repair is done to bring back normal movement to a joint. Tendon injury may occur anywhere in the body where there are tendons. The joints that are most commonly affected by tendon injuries are the shoulders, elbows, ankles, knees, and fingers. A tendon injury may occur from a laceration (cut) that goes past the skin and through the tendon. A tendon injury is also common from contact sports injuries such as football, wrestling, and rugby. Tendon damage can also occur in rheumatoid arthritis, an inflammatory disease of the joints. Rheumatoid arthritis can involve the tendons, causing them to tear. Generally, during tendon repair a surgeon will make one or more small incisions in the skin over the damaged tendon, sew the torn ends of the tendon together, check the surrounding tissue to make sure no other injuries have occurred, such as injury to the blood vessels or nerves, close the incision, cover the area with sterile bandages or dressings, and immobilize or splint the joint so as to allow the tendon to heal. If there is not enough healthy tendon to reconnect, the surgeon may perform a tendon graft using a piece of tendon from another part of the body. It may be from the foot or toe, for example. On occasion, a tendon transfer (moving a tendon from one area to another) may be useful in restoring function.

Other common soft-tissue surgeries of the lower extremity include tendon transfers, muscle repairs, fasciotomies, cartilage resections or repairs, and ligament reconstructions. Many of these surgeries are performed arthroscopically in an ambulatory surgery setting. However, in some cases, discharge may be delayed because of complications such as disruption of articular cartilage, menisci, and fat pads; damage to blood vessels, nerves, ligaments, and tendons; temporary paresis after tourniquet use; surgical instrument breakage; hemarthrosis; thrombophlebitis; and infection. To recover fully, physical therapy may be involved with functional activity progression and patient education before discharge.

Similarly, the mouth and throat include many soft tissues that require repair. Some of these oral surgeries could include a labial frenectomy, dental hemisection or root amputation, or soft tissue grafts to add more tissue in areas where gums have receded, the gum tissue is too thin, there is evidence of periodontal disease, an injury has affected the tissue, or where the roots of a tooth are exposed. Soft-tissue grafts are important procedures to maintain a healthy mouth, and can be used to prevent further gum recession, cover an exposed root, stop sensitivity in the affected area, improve the look of the tooth, or prevent future oral problems.

Peripheral artery disease (also called peripheral arterial disease) is a common circulatory problem in which narrowed arteries reduce blood flow to the limbs. When one develops peripheral artery disease (PAD), the extremities usually the legs do not receive enough blood flow to keep up with demand. This causes a number of symptoms. For example, in the case of PAD in the legs, the symptoms include leg pain when walking (claudication). Peripheral artery disease is also likely to be a sign of a more widespread accumulation of fatty deposits in the arteries (atherosclerosis). This condition may reduce blood flow to the heart and brain, as well as the limbs. Other conditions which can affect the peripheral vasculature include chronic venous insufficiency, deep vein thrombosis, and varicose veins.

### Peripheral Vascular Disease (PVD)

Peripheral vascular disease (PVD) is a blood circulation disorder that causes the blood vessels outside of the heart and brain to narrow, block, or spasm. This can happen in arteries or veins. PVD typically causes pain and fatigue, often in the legs, and especially during exercise. The pain usually improves with rest. It can also affect the vessels that supply blood and oxygen to arms, stomach and intestines, and kidneys. In PVD, blood vessels become narrowed and blood flow decreases. This can be due to arteriosclerosis, or “hardening of the arteries,” or it can be caused by blood vessel spasms. In arteriosclerosis, plaques build up in a vessel and limit the flow of blood and oxygen to organs and limbs. As plaque growth progresses, clots may develop and completely block the artery. This can lead to organ damage and loss of fingers, toes, or limbs, if left untreated. Peripheral arterial disease (PAD) develops only in the arteries, which carry oxygen-rich blood away from the heart. According to the CDC, approximately 12 to 20 percent of people over age 60 develop PAD, about 8.5 million people in the United States. PAD is the most common form of PVD, so the terms are often used to mean the same condition. PVD is also known as: arteriosclerosis obliterans; arterial insufficiency of the legs; Claudication; or intermittent claudication.

The two main types of PVD are functional and organic PVD. Functional PVD means there is no physical damage to blood vessels' structure. Instead, the vessels widen and narrow in response other factors like brain signals and temperature changes. The narrowing causes blood flow to decrease.

Organic PVD involves changes in blood vessel structure like inflammation, plaques, and tissue damage. Vessels naturally widen and narrow in response to environment. But in functional PVD, vessels exaggerate their response. Raynaud's disease, when stress and temperatures affect blood flow, is an example of functional PVD. The most common causes of functional PVD are emotional stress, cold temperatures, operating vibrating machinery or tools, or drugs. Organic PVD means there is change in the structure of blood vessels. For example, the plaque buildup from arteriosclerosis can cause blood vessels to narrow. The primary causes of organic PVD are smoking, high blood pressure, diabetes, or high cholesterol. Additional causes of organic PVD include extreme injuries, muscles or ligaments with abnormal structures, blood vessel inflammation, and infection.

There are numerous risk factors for PVD. Higher risk factors for PVD include being over age 50, being overweight, having abnormal cholesterol, having a history of cerebrovascular disease or stroke, having heart disease, having diabetes, having a family history of high cholesterol, high blood pressure, or PVD, having high blood pressure, or having kidney disease on hemodialysis. Lifestyle choices that can increase risk of developing PVD include not engaging in physical exercise, maintaining poor eating habits, smoking, or drug use.

For many people, the first signs of PVD begin slowly and irregularly. The patient may feel discomfort like fatigue and cramping in the legs and feet that gets worse with physical activity due to the lack of blood flow. Other symptoms of PVD include: in the legs reduced hair growth, cramps when lying in bed; in the legs and arms turn reddish blue or pale; in the legs and feet thin or pale skin, weak pulses, wounds, or ulcers that won't heal; in the toes a blue color, severe burning, or thick and opaque toe nails; or in the muscles feeling numb or heavy.

Symptoms of PVD are commonly brushed aside as the results of aging, but delayed diagnosis and treatment can cause further complications. In extreme cases of blood loss, gangrene, or dead tissue, can occur. If the patient suddenly develops a cold, painful, pale limb with weak or no pulses, this is a medical emergency. The patient will require treatment as soon as possible to avoid severe complications and amputation.

The most common symptom of PVD and PAD is claudication. Claudication is lower limb muscle pain when walking. The pain may be noticed especially when walking faster or for long distances. It usually goes away after some rest. When the pain comes back, it may take the same amount of time to go away. Claudication occurs when there is not enough blood flow to the muscles being used. In PVD, the narrowed vessels can only supply a limited amount of blood. This causes more problems during activity than at rest. As PAD progresses, symptoms will occur more frequently and get worse. Eventually, the patient may even experience pain and fatigue during rest.

Complications from undiagnosed and untreated PVD can be serious and even life-threatening. Restricted blood flow of PVD can be a warning sign of other forms of vascular disease. Complications of PVD can include: tissue death, which can lead to limb amputation; impotence; pale skin; pain at rest and with movement; severe pain that restricts mobility; wounds that don't heal; and life-threatening infections of the bones and blood stream. The most serious complications involve the arteries bringing blood to the heart and brain. When these become clogged, it can lead to heart attack, stroke, or death.

Early diagnosis is the first step to successful treatment and it can prevent life-threatening complications. Tell the doctor if any of the classic symptoms of PVD occur, such as claudication. The doctor will also ask about medical history and perform a physical exam. The physical exam can include measuring the pulses in legs and feet. If the doctor hears a whooshing sound through their stethoscope, it could mean a narrowed blood vessel. A doctor may order more specific tests to diagnose PVD. These tests may include Doppler ultrasound using sound waves for imaging to measure blood flow in vessels, ankle-brachial index (ABI) to use an ultrasound and blood pressure cuff around an ankle and/or arm, measured before and during exercise to measure a comparison of blood pressure readings in the leg and/or arm, as lower pressure in the leg could indicate a blockage, angiography using an injected dye in a catheter guided through the artery to measure the flow of dye through blood vessels to diagnose the clogged artery, magnetic resonance angiography (MRA) using magnetic field imaging to measure image of blood vessels to diagnose blockage, or computerized tomography angiography (CTA) using X-ray imaging to measure image of blood vessels to diagnose blockage.

The two main goals of PVD treatment are to stop the disease from progressing and to help manage pain and symptoms so the patient can remain active. The treatments will also lower risk for serious complications. First-line treatment typically involves lifestyle modifications. The doctor will suggest a regular exercise program that includes walking, a balanced diet, and losing weight. The patient should also quit smoking. Smoking directly causes reduced blood flow in vessels. It also causes PVD to get worse, as well as increasing risk of heart attack and stroke. If lifestyle changes alone are not enough, medication may be needed. Medications for PVD include cilostazol or pentoxifylline to increase blood flow and relieve symptoms of claudication, clopidogrel or daily aspirin to reduce blood clotting, atorvastatin, simvastatin, or other statins to lower high cholesterol, angiotensin-converting enzyme (ACE) inhibitors to lower high blood pressure, or diabetes medication to control blood sugar in diabetics. Significant artery blockages may require surgery like angioplasty or vascular surgery. Angioplasty is when the doctor inserts a catheter or long tube into the patient's artery. A balloon on the tip of the catheter inflates and opens up the artery. In some cases, the doctor will place a small wire tube in the artery, called a stent, to keep it open. Vascular surgery allows for blood to bypass the narrow area through vein grafting.

If diagnosed early, many cases of PVD will respond to lifestyle treatments. One way to measure improvement is to measure how far the patient can walk without pain. With effective treatment, the patient should be able to gradually increase the distance. The doctor should be contacted if symptoms get worse or if the patient experiences any of the following: legs look pale or blue; legs become cold; chest pain accompanies leg pain; legs become red, swollen, or hot; new sores or ulcers develop and do no heal; fever, chills, weakness, or other signs of infection.

Patients can reduce risk of developing PVD through a healthy lifestyle, which includes: avoiding smoking; controlling blood sugar in diabetics; setting an exercise goal of 30 minutes a day, five times a week; working to lower cholesterol and blood pressure; eating a healthy diet that's low in saturated fat; and keeping weight at a healthy level. Early diagnosis of PVD can help find ways to reduce symptoms and increase the effectiveness of treatment.

### Chronic Venous Insufficiency (CVI)

Chronic venous insufficiency (CVI) is a condition that occurs when the venous wall and/or valves in the leg veins are not working effectively, making it difficult for blood to return to the heart from the legs. CVI causes blood to “pool” or collect in these veins, and this pooling is called stasis.

Veins return blood to the heart from all the body's organs. To reach the heart, the blood needs to flow upward from the veins in the legs. Calf muscles and the muscles in the feet need to contract with each step to squeeze the veins and push the blood upward. To keep the blood flowing up, and not back down, the veins contain one-way valves.

Chronic venous insufficiency occurs when these valves become damaged, allowing the blood to leak backward. Valve damage may occur as the result of aging, extended sitting or standing or a combination of aging and reduced mobility. When the veins and valves are weakened to the point where it is difficult for the blood to flow up to the heart, blood pressure in the veins stays elevated for long periods of time, leading to CVI.

CVI most commonly occurs as the result of a blood clot in the deep veins of the legs, a disease known as deep vein thrombosis (DVT). CVI also results from pelvic tumors and vascular malformations, and sometimes occurs for unknown reasons. Failure of the valves in leg veins to hold blood against gravity leads to sluggish movement of blood out of the veins, resulting in swollen legs.

Chronic venous insufficiency that develops as a result of DVT is also known as post-thrombotic syndrome. As many as 30 percent of people with DVT will develop this problem within 10 years after diagnosis.

An estimated 40 percent of people in the United States have CVI. It occurs more frequently in people over age 50, and more often in women than in men.

The seriousness of CVI, along with the complexities of treatment, increase as the disease progresses. The earlier CVI is diagnosed and treated, the better the chance of preventing serious complications. Symptoms include: Swelling in the lower legs and ankles, especially after extended periods of standing; Aching or tiredness in the legs; New varicose veins; Leathery-looking skin on the legs; Flaking or itching skin on the legs or feet; or Stasis ulcers (or venous stasis ulcers). If CVI is not treated, the pressure and swelling increase until the tiniest blood vessels in the legs (capillaries) burst. When this happens, the overlying skin takes on a reddish-brown color and is very sensitive to being broken if bumped or scratched. At the least, burst capillaries can cause local tissue inflammation and internal tissue damage. At worst, this leads to ulcers, open sores on the skin surface. These venous stasis ulcers can be difficult to heal and can become infected. When the infection is not controlled, it can spread to surrounding tissue, a condition known as cellulitis. CVI is often associated with varicose veins, which are twisted, enlarged veins close to the surface of the skin. They can occur almost anywhere, but most commonly occur in the legs.

To diagnose CVI, the doctor will perform a complete medical history and physical exam. During the physical exam, the doctor will carefully examine the patient's legs. A test called a vascular or duplex ultrasound may be used to examine the blood circulation. During the vascular ultrasound, a transducer (small hand-held device) is placed on the skin over the vein to be examined. The transducer emits sound waves that bounce off the vein. These sound waves are recorded, and an image of the vessel is created and displayed on a monitor.

Like any disease, CVI is most treatable in its earliest stages. Vascular medicine or vascular surgery specialists typically recommend a combination of treatments for people with CVI. Some of the basic treatment strategies include: avoiding long periods of standing or sitting and if a long trip and will require sitting for a long time, flex and extend legs, feet, and ankles about 10 times every 30 minutes to keep the blood flowing in the leg veins; in the event that standing for long periods of time is required, then take frequent breaks to sit down and elevate feet; exercise regularly, walking is especially beneficial; lose weight if overweight; elevate legs while sitting and lying down, with legs elevated above the level of the heart; wear compression stockings; take antibiotics as needed to treat skin infections; and practice good skin hygiene. The goals of treatment are to reduce the pooling of blood and prevent leg ulcers.

The most conservative approach is to wear properly-fitting support hose (also called compression stockings). Compression stockings can be purchased at some pharmacies and medical supply stores and come in various styles, including below-the-knee, above-the-knee and pantyhose styles. They also come in different compressions varying from 8 to 10 mm Hg, up to 40 to 50 mm Hg. Compression stockings should be removed daily for washing and drying and to check the skin underneath. The stockings should fit so there is no bunching. Elastic stockings that fit poorly can actually make the condition worse by blocking blood flow in the area where they have bunched up. Some studies show that combining elastic socks with prescription medication to improve blood flow is very effective when the socks alone do not control symptoms.

Antibiotics may be prescribed to clear skin infections related to CVI, but the underlying disease must be treated to prevent a recurrence. Deeper infections and ulcers may also be treated with antibiotics.

For a patient with post-thrombotic syndrome, the doctor may prescribe medication to prevent the formation of additional blood clots.

A special medicated wrap, known as an Unna Boot, combines multilayer compression with a zinc oxide gel-based wound cover that forms a semi-rigid bandage. Other multilayer compression systems are available and are often used in combination with topical wound care products.

Some patients have found benefit from the herbal dietary supplement Vena-Stat, which contains a derivative of horse chestnut extract. Keep in mind that herbal preparations should not be used in place of prescription medications and should be used with caution, as they may interact with current prescription medications.

Skin should be kept moisturized so it does not flake or crack easily. If the skin is not broken or leaking fluid, but is inflamed, a doctor may recommend an anti-itch cream, such as one containing hydrocortisone; a cream containing zinc oxide to protect the skin; or an antifungal cream to prevent fungal infections. Skin leaking fluid is treated with wet compresses. In the case of leg ulcers, the doctor may recommend application of layered compression bandages to protect the skin and maintain blood flow.

Nonsurgical treatments include sclerotherapy and endovenous thermal ablation. Sclerotherapy involves the injection of a solution directly into spider veins or small varicose veins that causes them to collapse and disappear. Several sclerotherapy treatments are usually required to achieve the desired results. Sclerotherapy is simple, relatively inexpensive, and can be performed in the doctor's office. Sclerotherapy can eliminate the pain and discomfort of these veins and helps prevent complications such as venous hemorrhage and ulceration. It is also frequently performed for cosmetic reasons. Endovenous thermal ablation is a newer technique that uses a laser or high-frequency radio waves to create intense local heat in the affected vein. The technology is different with each energy source, but both forms of local heat close up the targeted vessel. This treatment closes off the problem veins but leaves them in place so there is minimal bleeding and bruising. Compared with ligation and stripping, endovenous thermal ablation results in less pain and a faster return to normal activities, with similar cosmetic results.

For the less than 10 percent of patients who require surgical treatment, the options include vein ligation and stripping, microincision/ambulatory phlebectomy, and bypass surgery. Here is a brief review of each of these techniques. Ligation and stripping often are performed in combination. Vein ligation is a procedure in which a vascular surgeon cuts and ties off the problem veins. Most patients recover in a few days and can resume their normal activities. Stripping is the surgical removal of larger veins through two small incisions. Stripping is a more extensive procedure and may require up to 10 days for recovery. It usually causes bruising for several weeks after surgery. Microincision/ambulatory phlebectomy is a minimally invasive procedure in which small incisions or needle punctures are made over the veins, and a phlebectomy hook is used to remove the problem veins. Vein bypass in the leg is similar to heart bypass surgery, just in a different location. It involves using a portion of healthy vein transplanted from elsewhere in the body to reroute blood around the vein affected by CVI. Bypass is used for treatment of CVI in the upper thigh and only in the most severe cases, when no other treatment is effective.

### Peripheral Arterial Disease (PAD)

Peripheral artery disease (also called peripheral arterial disease) is a common circulatory problem in which narrowed arteries reduce blood flow to limbs. When peripheral artery disease (PAD) is developed, the extremities usually legs do not receive adequate blood flow to keep up with demand. This causes symptoms, most notably leg pain when walking (claudication). PAD is also likely to be a sign of a more widespread accumulation of fatty deposits in the arteries (atherosclerosis). This condition may be reducing blood flow to heart and brain, as well as legs. PAD often can successfully be treated by quitting tobacco, exercising and eating a healthy diet.

While many people with peripheral artery disease have mild or no symptoms, some people have leg pain when walking (claudication). Claudication symptoms include muscle pain or cramping in legs or arms that is triggered by activity, such as walking, but disappears after a few minutes of rest. The location of the pain depends on the location of the clogged or narrowed artery. Calf pain is the most common location. The severity of claudication varies widely, from mild discomfort to debilitating pain. Severe claudication can make it hard for walking or doing other types of physical activities. PAD signs and symptoms include: painful cramping in one or more of hips, thighs or calf muscles after certain activities, such as walking or climbing stairs (claudication); leg numbness or weakness; coldness in lower leg or foot, especially when compared with the other side; sores on toes, feet or legs that will not heal; a change in the color of legs; hair loss or slower hair growth on feet and legs; slower growth of toenails; shiny skin on legs; no pulse or a weak pulse in legs or feet; and erectile dysfunction in men. If PAD progresses, pain may even occur when at rest or when lying down (ischemic rest pain). It may be intense enough to disrupt sleep. Hanging legs over the edge of the bed or walking around the room may temporarily relieve the pain.

PAD is often caused by atherosclerosis. In atherosclerosis, fatty deposits (plaques) build up on artery walls and reduce blood flow. Although discussions of atherosclerosis usually focus on the heart, the disease can and usually does affect arteries throughout the body. When it occurs in the arteries supplying blood to limbs, it causes PAD. Less commonly, the cause of PAD may be blood vessel inflammation, injury to limbs, unusual anatomy of ligaments or muscles, or radiation exposure.

If PAD is caused by a buildup of plaques in blood vessels (atherosclerosis), there are also risks of developing: Critical limb ischemia, which begins as open sores that do not heal, an injury, or an infection of feet or legs. Critical limb ischemia occurs when such injuries or infections progress and cause tissue death (gangrene), sometimes requiring amputation of the affected limb; or stroke and heart attack. The atherosclerosis that causes the signs and symptoms of peripheral artery disease is not limited to legs. Fat deposits also build up in arteries supplying blood to the heart and brain.

Some of the tests used to diagnose PAD are:

A physical examination may uncover a weak or absent pulse below a narrowed area of a patient's artery, whooshing sounds (bruits) over arteries that can be heard with a stethoscope, evidence of poor wound healing in the area where blood flow is restricted, and decreased blood pressure in an affected limb.

Ankle-brachial index (ABI) is a common test used to diagnose PAD. It compares the blood pressure in the patient's ankle with the blood pressure in the patient's arm.

To get a blood pressure reading, the doctor uses a regular blood pressure cuff and a special ultrasound device to evaluate blood pressure and flow.

Walking on a treadmill and have readings taken before and immediately after exercising to capture the severity of the narrowed arteries during walking.

Special ultrasound imaging techniques, such as Doppler ultrasound, can help evaluate blood flow through blood vessels and identify blocked or narrowed arteries.

Angiography. Using a dye (contrast material) injected into blood vessels, this test allows viewing of blood flow through arteries as it happens. A doctor is able to trace the flow of the contrast material using imaging techniques, such as X-ray imaging or procedures called magnetic resonance angiography (MRA) or computerized tomography angiography (CTA).

Catheter angiography is a more invasive procedure that involves guiding a catheter through an artery in the groin to the affected area and injecting the dye that way. Although invasive, this type of angiography allows for simultaneous diagnosis and treatment. After finding the narrowed area of a blood vessel, the doctor can then widen it by inserting and expanding a tiny balloon or by administering medication that improves blood flow.

Blood tests. A sample of blood can be used to measure cholesterol and triglycerides and to check for diabetes.

Treatment for peripheral artery disease has two major goals: 1) Manage symptoms, such as leg pain, so that physical activities can be resumed; 2) Stop the progression of atherosclerosis throughout the body to reduce risk of heart attack and stroke. These goals may be accomplished with lifestyle changes, especially early in the course of peripheral artery disease. Quitting smoking is the single most important thing to reduce risk of complications. If signs or symptoms of PAD exist, additional medical treatment may be needed. Medicine may be prescribed to prevent blood clots, lower blood pressure and cholesterol, and/or control pain and other symptoms.

Cholesterol-lowering medications. Statins may reduce risk of heart attack and stroke. The goal for people who have PAD is to reduce low-density lipoprotein (LDL) cholesterol, the “bad” cholesterol, to less than 100 milligrams per deciliter (mg/dL), or 2.6 millimoles per liter (mmol/L). The goal is even lower if additional major risk factors for heart attack and stroke are present, especially diabetes or continued smoking.

High blood pressure medications. The doctor may prescribe medications to lower high blood pressure. A blood pressure treatment goal should be less than 130/80 mm Hg. This is the guideline for anyone with coronary artery disease, diabetes or chronic kidney disease. Achieving 130/80 mm Hg is also the goal for healthy adults age 65 and older and healthy adults younger than age 65 with a 10 percent or higher risk of developing cardiovascular disease in the next 10 years.

Medication to control blood sugar. For diabetics it becomes even more important to control blood sugar (glucose) levels.

Medications to prevent blood clots. Because PAD is related to reduced blood flow to limbs, it's important to improve that flow. Daily aspirin therapy or another medication, such as clopidogrel (Plavix) may be prescribed.

Symptom-relief medications. The drug cilostazol increases blood flow to the limbs both by keeping the blood thin and by widening the blood vessels. It specifically helps treat symptoms of claudication, such as leg pain, for people who have peripheral artery disease. Common side effects of this medication include headache and diarrhea. An alternative to cilostazol is pentoxifylline. Side effects are rare with this medication, but it's generally less effective than cilostazol.

In some cases, angioplasty or surgery may be necessary to treat peripheral artery disease that is causing claudication: Angioplasty. In this procedure, a small hollow tube (catheter) is threaded through a blood vessel to the affected artery. There, a small balloon on the tip of the catheter is inflated to reopen the artery and flatten the blockage into the artery wall, while at the same time stretching the artery open to increase blood flow. The doctor may also insert a mesh framework called a stent in the artery to help keep it open. This is the same procedure doctors use to open heart arteries.

Bypass surgery. The doctor may create a graft bypass using a vessel from another part of the body or a blood vessel made of synthetic (man-made) fabric. This technique allows blood to flow around or bypass the blocked or narrowed artery. Thrombolytic therapy. If a blood clot is blocking an artery, the doctor may inject a clot-dissolving drug into the artery at the point of the clot to break it up.

In addition to medications or surgery, the doctor likely will prescribe a supervised exercise training program to increase the distance that can be walked pain-free. Regular exercise improves symptoms of PAD in a number of ways, including helping the patient's body use oxygen more efficiently.

### Deep Vein Thrombosis (DVT)

Deep vein thrombosis (DVT) occurs when a blood clot (thrombus) forms in one or more of the deep veins in the body, usually in legs. Deep vein thrombosis can cause leg pain or swelling, but also can occur with no symptoms. Deep vein thrombosis can develop if the patient has certain medical conditions that affect how blood clots. It can also happen if the patient does not move for a long time, such as after surgery or an accident, or when confined to bed. Deep vein thrombosis can be very serious because blood clots in veins can break loose, travel through the bloodstream and lodge in lungs, blocking blood flow (pulmonary embolism).

Deep vein thrombosis signs and symptoms can include: 1) Swelling in the affected leg. Rarely, there may be swelling in both legs. 2) Pain in leg. The pain often starts in the calf and can feel like cramping or soreness. 3) Red or discolored skin on the leg. A feeling of warmth in the affected leg. Deep vein thrombosis can occur without noticeable symptoms.

The blood clots of deep vein thrombosis can be caused by anything that prevents blood from circulating or clotting normally, such as injury to a vein, surgery, certain medications and limited movement.

To diagnose deep vein thrombosis, the doctor will ask about symptoms. The doctor may perform a physical examination to check for areas of swelling, tenderness or discoloration on the skin. Depending the likelihood of a clot, the doctor might suggest tests, including: 1) ultrasound; 2) blood test; 3) venography; or 4) CT or MRI scans. In an ultrasound, a wandlike device (transducer) placed over the part of the body where there is a clot sends sound waves into the area. As the sound waves travel through tissue and reflect back, a computer transforms the waves into a moving image on a video screen. A clot might be visible in the image. Sometimes a series of ultrasounds are done over several days to determine whether a blood clot is growing or to check for a new one. In a blood test almost all people who develop severe deep vein thrombosis have an elevated blood level of a substance called D dimer. In a venography a dye is injected into a large vein in the foot or ankle. An X-ray creates an image of the veins in legs and feet, to look for clots. However, less invasive methods of diagnosis, such as ultrasound, can usually confirm the diagnosis. CT or MRI scans can provide visual images of veins and might show if a clot exists. Sometimes these scans performed for other reasons reveal a clot.

Deep vein thrombosis (DVT) treatment is aimed at preventing the clot from getting bigger and preventing it from breaking loose and causing a pulmonary embolism. Then the goal becomes reducing chances of deep vein thrombosis happening again. Deep vein thrombosis treatment options include: 1) blood thinners; 2) clot busters; 3) filters; or 4) compression stockings.

Deep vein thrombosis is most commonly treated with anticoagulants, also called blood thinners. These drugs, which can be injected or taken as pills, decrease the blood's ability to clot. They do not break up existing blood clots, but they can prevent clots from getting bigger and reduce risk of developing more clots. The injectable medications can be given as a shot under the skin or by injection into an arm vein (intravenous). Heparin is typically given intravenously. Other similar blood thinners, such as enoxaparin (Lovenox), dalteparin (Fragmin) or fondaparinux (Arixtra), are injected under the skin. An injectable blood thinner might be taken for a few days, after which pills such as warfarin (Coumadin, Jantoven) or dabigatran (Pradaxa) are started. Once warfarin has thinned the blood, the injectable blood thinners are stopped. Other blood thinners can be given in pill form without the need for an injectable blood thinner. These include rivaroxaban (Xarelto), apixaban (Eliquis) or edoxaban (Savaysa). Blood thinner pills then might be taken for three months or longer. It is important to take them exactly as instructed because taking too much or too little can cause serious side effects. If warfarin is taken, periodic blood tests will be needed to check how long it takes blood to clot. Pregnant women should not take certain blood-thinning medications.

Sometimes clot buster drugs are needed. If a patient has a more serious type of deep vein thrombosis or pulmonary embolism, or if other medications are not working, the doctor might prescribe drugs that break up clots quickly, called clot busters or thrombolytics. These drugs are either given through an IV line to break up blood clots or through a catheter placed directly into the clot. These drugs can cause serious bleeding, so they're generally reserved for severe cases of blood clots.

If medications cannot be taken to thin the blood, a patient might have a filter inserted into a large vein the vena cava in the abdomen. A vena cava filter prevents clots that break loose from lodging in the lungs.

To help prevent swelling associated with deep vein thrombosis, compression stockings can be worn on legs from feet to about the level of knees. This pressure helps reduce the chances that blood will pool and clot. These stockings should be worn during the day for at least two years, if possible.

### Varicose Veins

Varicose veins are swollen, twisted veins that can be seen just under the surface of the skin. These veins usually occur in legs, but they also can form in other parts of the body. Varicose veins are a common condition. They usually cause few signs and symptoms. Sometimes varicose veins cause mild to moderate pain, blood clots, skin ulcers (sores), or other problems.

Veins have one-way valves that help keep blood flowing toward the heart. If the valves are weak or damaged, blood can back up and pool in the veins. This causes the veins to swell, which can lead to varicose veins. Many factors can raise the risk for varicose veins. Examples of these factors include family history, older age, gender, pregnancy, overweight or obesity, lack of movement, and leg trauma. Varicose veins are treated with lifestyle changes and medical procedures. The goals of treatment are to relieve symptoms, prevent complications, and improve appearance. Varicose veins usually do not cause medical problems. If they do, the doctor may simply suggest making lifestyle changes. Sometimes varicose veins cause pain, blood clots, skin ulcers, or other problems. If this happens, the doctor may recommend one or more medical procedures. Some people choose to have these procedures to improve the way their veins look or to relieve pain. Many treatments for varicose veins are quick and easy and do not require a long recovery.

Lifestyle changes often are the first treatment for varicose veins. These changes can prevent varicose veins from getting worse, reduce pain, and delay other varicose veins from forming. Lifestyle changes include the following: 1) Avoid standing or sitting for long periods without taking a break. When sitting, avoid crossing legs. Keep legs raised when sitting, resting, or sleeping. When possible, raise legs above the level of the heart. 2) Do physical activities to get legs moving and improve muscle tone. This helps blood move through veins. 3) Lose weight if overweight or obese. This will improve blood flow and ease the pressure on the veins. 4) Avoid wearing tight clothes, especially those that are tight around the waist, groin (upper thighs), and legs. Tight clothes can make varicose veins worse. 5) Avoid wearing high heels for long periods. Lower heeled shoes can help tone the calf muscles. Toned muscles help blood move through the veins.

The doctor may recommend compression stockings. These stockings create gentle pressure up the leg. This pressure keeps blood from pooling and decreases swelling in the legs. There are three types of compression stockings. One type is support pantyhose. These offer the least amount of pressure. A second type is over-the-counter compression hose. These stockings give a little more pressure than support pantyhose. Over-the-counter compression hose are sold in medical supply stores and pharmacies. Prescription-strength compression hose are the third type of compression stockings. These stockings offer the greatest amount of pressure. They also are sold in medical supply stores and pharmacies. However, the patient need to be fitted for them in the store by a specially trained person.

Medical procedures are done either to remove varicose veins or to close them. Removing or closing varicose veins usually do not cause problems with blood flow because the blood starts moving through other veins.

The patient may be treated with one or more of the procedures described below. Common side effects right after most of these procedures include bruising, swelling, skin discoloration, and slight pain.

The side effects are most severe with vein stripping and ligation. Rarely, this procedure can cause severe pain, infections, blood clots, and scarring.

Sclerotherapy uses a liquid chemical to close off a varicose vein. The chemical is injected into the vein to cause irritation and scarring inside the vein. The irritation and scarring causes the vein to close off, and it fades away. This procedure often is used to treat smaller varicose veins and spider veins. It can be done in a doctor's office, while the patient is standing. The patient may need several treatments to completely close off a vein. Treatments typically are done every 4 to 6 weeks. Following treatments, the patient's legs will be wrapped in elastic bandaging to help with healing and decrease swelling.

Microsclerotherapy is used to treat spider veins and other very small varicose veins. A small amount of liquid chemical is injected into a vein using a very fine needle. The chemical scars the inner lining of the vein, causing it to close off.

Laser Surgery procedure applies light energy from a laser onto a varicose vein. The laser light makes the vein fade away. Laser surgery mostly is used to treat smaller varicose veins. No cutting or injection of chemicals is involved.

Endovenous Ablation Therapy uses lasers or radiowaves to create heat to close off a varicose vein. The doctor makes a tiny cut in the skin near the varicose vein. He or she then inserts a small tube called a catheter into the vein. A device at the tip of the tube heats up the inside of the vein and closes it off. The patient will be awake during this procedure, but the doctor will numb the area around the vein.

For Endoscopic Vein Surgery the doctor will make a small cut in the patient's skin near a varicose vein. The doctor will then uses a tiny camera at the end of a thin tube to move through the vein. A surgical device at the end of the camera is used to close the vein. Endoscopic vein surgery usually is used only in severe cases when varicose veins are causing skin ulcers (sores).

For Ambulatory Phlebectomy, the doctor will make small cuts in the patient's skin to remove small varicose veins. This procedure usually is done to remove the varicose veins closest to the surface of the skin. The patient be awake during the procedure, but the doctor will numb the area around the vein.

Vein Stripping and Ligation typically is done only for severe cases of varicose veins. The procedure involves tying shut and removing the veins through small cuts in the skin. The patient will be given medicine to temporarily sleep so as not to feel any pain during the procedure. Vein stripping and ligation usually is done as an outpatient procedure. The recovery time from the procedure is about 1 to 4 weeks.

### Stress Urinary Incontinence

The urinary tract system includes two kidneys, two ureters, a bladder, a urethra and a sphincter. The kidneys clean the blood and remove waste and excess water in the form of urine. The kidneys also serve as the body's filter to control electrolytes, fluid balance, pH and blood pressure. Urine drains from the kidneys down through thin tubes called ureters into the bladder. The bladder is a balloon-like organ that stores the urine and is secured in place by fascia in the pelvic floor. The bladder muscles contract to release urine through the urethra. The urethra is a tube at the bottom of the bladder where urine exits the body. It has sphincter muscles to keep the urethra closed and prevent urine from leaking out. The sphincter muscles relax when the bladder contracts and urination occurs. The pelvic floor includes a sling (like a hammock) of muscles and fascia that supports the bladder, and rectum (and the uterus in females).

Stress urinary incontinence (SUI) is a condition where sudden pressure on the bladder and urethra causes the sphincter muscles to open briefly and leak urine. Any activity that increases abdominal pressure could lead to SUI leakage. With mild SUI, pressure may be from sudden forceful activities, like exercise, sneezing, laughing or coughing. With moderate to more severe SUI, leakage may occur with less forceful activities like standing up, walking or bending over. Urinary “accidents” can range from a few drops of urine to enough urine to soak through clothes. SUI is a common bladder problem for women, but happens less often in men.

As mentioned above, the pelvic floor supports the bladder and urethra. If this area gets stretched, weakened or damaged, then SUI can happen. In women, pregnancy and childbirth can be a cause of this type of stretching of the pelvic floor that leads to SUI. Chronic coughing or nerve injuries to the lower back or pelvic surgery (like surgery for prostate cancer) can also weaken the muscles in the pelvic floor and lead to SUI.

Another common bladder problem is called Overactive Bladder (OAB), or Urgency Urinary Incontinence (UUI). People with OAB have an urgent, uncontrollable “gotta go” need to urinate that could happen quite often. Some people with OAB leak urine. The difference between SUI and OAB is anatomical. SUI is a urethral problem while OAB is a bladder problem. With SUI, the urethra cannot stop the sudden increase in pressure. With OAB, the bladder spasms and squeezes uncontrollably. Many people with SUI also have OAB. When both types of urinary incontinence are happening, it is called “Mixed Incontinence.” About 1 in 3 women suffer from SUI at some point in their lives. Urinary incontinence increases with age. Over half of women with SUI also have OAB. About one-third (1 out of 3) of women age 60 find that they sometimes leak urine. About half (1 out of 2) of women age 65 and above find that they sometimes leak urine. Men with urine leakage have overactive bladder (OAB) more often than SUI. For men who have SUI, it is likely due to prostate cancer surgery, pelvic nerve injury or damage.

Risk factors for SUI include: gender, females are more likely to get SUI; pregnancy and childbirth; being overweight; smoking; chronic coughing; nerve injuries to the lower back, and pelvic or prostate surgery. For women, a physical exam to diagnose SUI may include checking the abdomen, the organs in the pelvis, and the rectum. For men, a physical exam may include checking genitalia and abdomen, prostate and rectum. A healthcare provider may also test how strong the pelvic floor muscles and sphincter muscles are. (One way of testing the strength of pelvic and sphincter muscles may be through a Kegel test.) Another way of diagnosing SUI may ask the patient to perform maneuvers such as coughing, straining down or stepping to see if these actions cause urine leakage.

One tool used for diagnosing SUI is a “bladder diary.” A bladder diary is used to track day-to-day symptoms, and should include both what fluids are drunk and how often each trip to the bathroom occurs. The bladder diary should also include each time a leakage of urine occurs and what activities (for example, running, coughing or sneezing) where being performed when the leak happened.

Another type of test used to diagnose SUI is a urinary pad test. There are two types of urinary pad tests: the one-hour test and the 24-hour test. The one-hour pad test is usually done in a doctor's office to learn about leakage with exercise or movement. The pad is removed and weighed afterwards to evaluate the amount of urine leaked. The 24-hour urine pad test is usually done at home for a complete day and night evaluation.

Additional types of tests may be used when a patient presents with symptoms of SUI. These tests might include, for example, a urinalysis or urine sample to test for a urinary tract infection or blood in the urine; a bladder scan after urinating to show how much urine stays in the bladder after urinating; a cystoscopy, which uses a narrow tube with a tiny camera to see into the bladder to rule out more serious problems; or urodynamic studies (UDS) are done to test how well the bladder, sphincters and urethra hold and release urine.

SUI can often be treated either as an alternative or a combination therapy with lifestyle changes, exercise, devices or surgery. There are currently no drugs approved in the U. S. to treat SUI. In the case of mixed incontinence (both SUI and OAB) a physician may prescribe OAB drugs. These drugs may help reduce leaks for OAB, but will not help with SUI.

Non-surgical options for SUI may include using absorbent pads, which come in different size and can be worn in underwear or are integrated in pull-on briefs; performing pelvic floor muscle exercises (Kegel exercises) to strengthen pelvic floor muscles, which help support the bladder and other organs and may help reduce or eliminate SUI symptoms; maintaining of good bowel function and prevention of constipation to encourage regular bowel movements by eating high fibers foods, drinking adequate water and exercising daily; maintaining a healthy weight; training the bladder using a bladder diary to methodically lengthen the time between bathroom visits by a fixed schedule; or quitting smoking.

Other non-surgical options for females may include use of an inserted vaginal device such as tampons, over-the-counter pessaries and custom fitted pessaries. Some women find that inserting a simple tampon during exercise prevents leaks. However, tampons have not been approved for this purpose, and there is no research that shows tampons can prevent urinary leakage. A vaginal pessary is a firm yet flexible device that is inserted into the vagina. It repositions and supports the urethra and/or uterus. A single use, disposable pessary is available over-the-counter without a prescription. The device is inserted with an applicator, like a tampon. Once the pessary is in the vagina, the core and cover of the device support the urethra. Disposable devices are made to be used for a maximum of 8 hours in a 24-hour period. All pessaries have some risk of irritation or infection. These devices typically press against the wall of the vagina and the urethra. The pressure helps reposition and support the urethra, which leads to fewer leaks with minimal risk. Prescription pessaries are small, often made of medical grade silicone. These must be fitted by a specialist. Like other pessaries, they are inserted into the vagina and the pelvic floor muscles hold it in place. When fitted properly, it should not be noticeable to the wearer and can allow for normal daily activities. Some women wear the pessary 24 hours a day, but most women wear them during the day and remove them at night. The pessary must be removed before having sexual intercourse. Constant wearing of a pessary can irritate the urethra, which could lead to blood in the urine (hematuria) and urinary tract infections. Pessaries are generally safe, with a small risk of infection, and they are useful for reducing leaks during strenuous activities like running, lifting or playing tennis.

Another non-surgical option for preventing SUI in women during periods of significant activity may include use of an occlusive device (also called urethral plug). A urethral plug will serve to block the urethra, while a vaginal device adds support through the vagina. A simple urethral plug can be inserted to create a barrier. They may be shaped like a thin flexible rod. Some have a balloon on the end that can be inflated and deflated to block leaks and when it is time to urinate, the device can be deflated or pulled out. These plugs are used only in rare and specific cases. Currently, there are no approved urethral plugs available in the United States.

Non-surgical options to prevent SUI leaks in men may include a penile clamp/clip device. These external clamps may be used to restrict the flow of urine from the penis.

Surgical options to address SUI are provided based upon the severity of symptoms of incontinence and patent preferences. Surgery for SUI in women is usually very successful. Here are some common surgical options:

First, urethral injections may be used to “bulk up” the urethral sphincter muscle that keeps the urethra closed. In this type of procedure, “bulking agents” are injected into the urethra, which helps the sphincter to close the bladder better. Often, the injections are done under local anesthesia in a physician's office. Bulking agents are a temporary treatment for SUI. Of every 10 women who have these injections, between 1 in 3 are cured of leaks, which can last for a year. This procedure may not be as effective as other surgeries, but the recovery time is short and the injections can be repeated as needed.

Second, the most common surgery for SUI in women is “sling” surgery. In this procedure, a small strip of material (a sling) is placed under the urethra to prevent it from moving downward during activities. It acts as a hammock to support the urethra. Many sling techniques and materials have been developed. Slings can be made from patient tissue, donor tissue or surgical mesh.

“Midurethral sling” is the most common type of surgery used to correct SUI. The sling is made out of a narrow strip of synthetic mesh that is placed under the urethra with a variety of techniques: retropubic, transobturator and single-incision. For sling surgery made from surgical mesh, the surgeon may only need to make a small incision in the vagina. The sling is then inserted under the urethra and anchored in the surrounding pelvic floor tissue. This surgery is short and recovery may be shorter than with an autologous sling.

“Autologous sling” is another type of surgery to correct SUI. The sling is made from a strip of the patient's own tissue (autologous) taken from the lower abdomen or thigh. The ends of the sling are stitched in place through an incision in the abdomen. To use the patient's body tissue for a sling, an additional incision is made in the lower belly or in the thigh to collect tissue that will used for the sling. A specialist may be needed to provide this option (as it is not as common as mid-urethral synthetic sling surgery). Autologous sling surgery is usually done through a cut in the bikini line. Or it can be done making a cut over the thigh. The surgery is most often done in less than 2 hours. This surgery does require more time to recover than a mid-urethral sling surgery. There are additional risks associated with this type of surgery.

Bladder Neck Suspension surgery is also called Retropubic Suspension, Colposuspension or Burch Suspension. This procedure is used when the bladder or urethra has fallen out of its normal position. Sutures are placed in the tissue along the side of the bladder neck and urethra and attached to a ligament along the pubic bone. This supports the urethra and sphincter muscles to prevent them from moving downward and accidentally opening. The procedure adds support to the bladder neck and urethra, reducing the risk of stress incontinence. The surgery can be done open or laparoscopically under general anesthesia in less than a few hours. However, it requires more time to recover than mid-urethral sling surgery.

A number of surgical options exist for men with SUI. The most effective treatment for male SUI is to implant an artificial urinary sphincter device, which has three parts: a fluid-filled cuff (the artificial sphincter), surgically placed around the urethra; a fluid-filled, pressure-regulating balloon, inserted into the belly; and a controllable pump inserted into the scrotum. The artificial urinary sphincter cuff is filled with fluid, which keeps the urethra closed and prevents leaks. When the pump is pressed, the fluid in the cuff is transferred to the balloon reservoir. This opens the urethra allowing urination. Once urination is complete, the balloon reservoir automatically refills the urethral cuff in 1-3 minutes. Artificial sphincter surgery can cure or greatly improve urinary control in more than 7 out of 10 men with SUI. Results may vary in men who have had radiation treatment. They also vary in men with other bladder conditions or who have scar tissue in the urethra.

Another surgical option for men is implanting a sling. Similar to female mid-urethral slings, the male sling is a narrow strap made of synthetic mesh that is placed under the urethra. It acts as a hammock to lift and support the urethra and sphincter muscles. Most commonly, slings for men are made of surgical mesh. The surgical incision to place the sling is between the scrotum and rectum. The male sling is most often used in men with mild to moderate SUI. It is less effective in men who have had radiation therapy to the prostate or urethra, or men with severe incontinence.

What is needed in the art are treatment protocols and compositions for stabilization of the organs and tissues affected by conditions such as SUI.

### Congestive Heart Failure

Congestive heart failure occurs when the heart muscle does not pump blood as well as it should. Certain conditions, such as narrowed arteries in the heart (coronary artery disease) or high blood pressure, gradually leave the heart too weak or stiff to fill and pump efficiently. Heart failure can be chronic or acute. Symptoms may include dyspnea, fatigue, weakness, edema in the legs, ankles and feet, rapid or irregular heartbeat, reduced ability to exercise, persistent cough or wheezing, increased need to urinate at night, ascites, rapid weight gain from fluid retention, lack of appetite, nausea, difficulty concentrating, decreased alertness, and sudden, severe shortness of breath.

Heart failure often develops after other conditions have damaged or weakened the heart. However, the heart doesn't need to be weakened to cause heart failure. It can also occur if the heart becomes too stiff.

In heart failure, the main pumping chambers of the heart (the ventricles) may become stiff and not fill properly between beats. In some cases of heart failure, the heart muscle may become damaged and weakened, and the ventricles stretch (dilate) to the point that the heart can't pump blood efficiently throughout the body.

Over time, the heart can no longer keep up with the normal demands placed on it to pump blood to the rest of the body. An ejection fraction is an important measurement of how well the heart is pumping and is used to help classify heart failure and guide treatment. In a healthy heart, the ejection fraction is 50 percent or higher—meaning that more than half of the blood that fills the ventricle is pumped out with each beat. But heart failure can occur even with a normal ejection fraction. This happens if the heart muscle becomes stiff from conditions such as high blood pressure. Heart failure can involve the left side (left ventricle), right side (right ventricle) or both sides of the heart. Generally, heart failure begins with the left side, specifically the left ventricle—the heart's main pumping chamber.

In left-sided hearth failure, fluid may back up in the lungs, causing shortness of breath. In right-sided heart failure, fluid may back up into the abdomen, legs, and feet, causing swelling. In systolic heart failure, the left ventricle can't contract vigorously, indicating a pumping problem. In diastolic heart failure (heart failure with preserved ejection fraction), the left ventricle cannot relax or fill fully, indicating a filling problem.

A number of conditions can cause heart failure. These include coronary artery disease and heart attack. Coronary artery disease is the most common form of heart disease and the most common cause of heart failure. The disease results from the buildup of fatty deposits (plaque) in the arteries, which reduce blood flow and can lead to heart attack. High blood pressure (hypertension) causes the heart has to work harder than it should to circulate blood throughout the body. Over time, this extra exertion can make the heart muscle too stiff or too weak to effectively pump blood. The valves of the heart keep blood flowing in the proper direction through the heart. A damaged valve—due to a heart defect, coronary artery disease or heart infection—forces the heart to work harder, which can weaken it over time. Damage to the heart muscle (cardiomyopathy) can have many causes, including several diseases, infections, alcohol abuse and the toxic effect of drugs, such as cocaine or some drugs used for chemotherapy. Genetic factors also can play a role. Myocarditis is an inflammation of the heart muscle. It is most commonly caused by a virus and can lead to left-sided heart failure. Congenital heart defects, where the heart and its chambers or valves haven't formed correctly, causes the healthy parts of the heart to have to work harder to pump blood through the heart, which, in turn, may lead to heart failure. Abnormal heart rhythms (heart arrhythmias) may cause the heart to beat too fast, creating extra work for the heart. A slow heartbeat also may lead to heart failure. Chronic diseases—such as diabetes, HIV, hyperthyroidism, hypothyroidism, or a buildup of iron (hemochromatosis) or protein (amyloidosis)—also may contribute to heart failure. Causes of acute heart failure include viruses that attack the heart muscle, severe infections, allergic reactions, blood clots in the lungs, the use of certain medications or any illness that affects the whole body.

Risk factors for congestive heart failure include high blood pressure, coronary artery disease, heart attack, diabetes, certain diabetes medications (rosiglitazone, pioglitazone), certain medications (nonsteroidal anti-inflammatory drugs (NSAIDs), certain anesthesia medications; some anti-arrhythmic medications, certain medications used to treat high blood pressure, cancer, blood conditions, neurological conditions, psychiatric conditions, lung conditions, urological conditions, inflammatory conditions and infections), sleep apnea, congenital heart defects, valvular heart disease, viruses, alcohol use, tobacco use, obesity, and irregular heartbeats.

Complications of congestive heart failure can include kidney damage or failure. Heart failure can reduce the blood flow to the kidneys, which can eventually cause kidney failure if left untreated. Kidney damage from heart failure can require dialysis for treatment. The valves of the heart, which keep blood flowing in the proper direction through the heart, may not function properly if the heart is enlarged or if the pressure in the heart is very high due to heart failure. Heart rhythm problems (arrhythmias) can be a potential complication of heart failure. Heart failure can lead to a buildup of fluid that puts too much pressure on the liver. This fluid backup can lead to scarring, which makes it more difficult for the liver to function properly resulting in liver damage.

Conditions characterized by enzymatic degradation of structural proteins include Marfan syndrome, aneurysm, and supravalvular aortic stenosis. For those afflicted, such conditions lead to, at the very least, a lowered quality of life and often, premature death. Modifying protein degradation in cardiac tissue may be a valuable therapeutic approach to treatment of congestive heart failure. Global inhibition of protein degradation systems can alleviate disease progression in cardiomyopathies, the ability to modulate these processes more selectively offers huge potential for the developing novel therapeutics, while avoiding negative side-effects.

### Mitral Valve Repair or Replacement Procedures

The mitral valve regulates the flow of blood from the upper-left chamber (the left atrium) to the lower-left chamber (the left ventricle). Diseases of the heart valves are grouped according to which valve or valves are involved and the amount of blood flow that is disrupted by the problem. The most common and serious valve problems happen in the mitral and aortic valves. The mitral valve regulates the flow of blood from the upper-left chamber (the left atrium) to the lower-left chamber (the left ventricle). Three diseases of the mitral valve are mitral valve prolapse, mitral regurgitation, and mitral stenosis.

Mitral valve prolapse (MVP) means that one or both of the valve flaps (called cusps or leaflets) are enlarged, and the flaps' supporting muscles are too long. Instead of closing evenly, one or both of the flaps collapse or bulge into the left atrium. MVP is often called click-murmur syndrome because when the valve does not close properly, it makes a clicking sound and then a murmur. MVP is one of the most common forms of valve disease. It also runs in families. Some forms of MVP have been associated with Marfan syndrome, a connective tissue condition where patients have long bones and very flexible joints. Most people with MVP are small-framed or have minor chest wall deformities, scoliosis, or other skeletal disorders.

Mitral regurgitation is also called mitral insufficiency or mitral incompetence. It happens when the mitral valve allows a backflow of blood into the heart's upper-left chamber (the left atrium). Mitral regurgitation may take years to reveal itself. But, if it goes on long enough, it can cause a buildup of pressure in the lungs or cause the heart to enlarge. In time, this will lead to symptoms. Mitral regurgitation is usually caused by conditions that weaken or damage the valve.

Mitral stenosis is a narrowing or blockage of the mitral valve. The narrowed valve causes blood to backup in the heart's upper-left chamber (the left atrium) instead of flowing into the lower-left chamber (the left ventricle). Most adults with mitral stenosis had rheumatic fever when they were younger. Mitral stenosis may also be associated with aging and a buildup of calcium on the ring around the valve where the leaflet and heart muscle meet. Mitral stenosis is usually caused by rheumatic fever, but it can be caused by any condition that causes narrowing of the mitral valve. The condition is rarely passed down through family members.

Mitral valve repair and mitral valve replacement procedures may be performed to treat diseases of the mitral valve the valve located between the left heart chambers (left atrium and left ventricle). Several types of mitral valve disease exist. In mitral valve regurgitation, the flaps (leaflets) of the mitral valve do not close tightly, causing blood to leak backward into the left atrium. This commonly occurs due to valve leaflets bulging back—a condition called mitral valve prolapse. In another condition, called mitral valve stenosis, the leaflets become thick or stiff, and they may fuse together; this results in a narrowed valve opening and reduced blood flow through the valve. Treatment for mitral valve disease depends on the severity of the condition. Doctors may recommend surgery to repair or replace mitral valves for some people with mitral valve disease. Several surgical procedures exist to repair or replace mitral valves, including open-heart surgery or minimally invasive heart surgery.

Mitral valve disease treatment depends on how severe the patient's condition is, if the patient is experiencing signs and symptoms, and if the patient's condition is getting worse. The doctor and treatment team will evaluate the patient to determine the most appropriate treatment for the condition. In the evaluation, the doctor may conduct a physical examination, review medical history and perform tests. If symptoms are not being experienced or if the condition is mild, the doctor may first suggest monitoring the condition with regular evaluations. Medications may be prescribed to manage symptoms and if the condition is mild, no surgery may be needed. Even so, the mitral valve may eventually need to be repaired or replaced. In some cases, doctors may recommend mitral valve repair or mitral valve replacement even if a patient is not experiencing symptoms. Research has found that performing surgery in a person with severe mitral valve regurgitation who is not experiencing symptoms, rather than monitoring the condition, can improve long-term outcomes.

If heart surgery is needed for another condition in addition to mitral valve disease, doctors may conduct surgery to treat both conditions simultaneously. Doctors often may recommend mitral valve repair to address mitral valve disease. However, if mitral valve repair is not possible, doctors may need to perform mitral valve replacement. Doctors may also evaluate whether a patient is a candidate for minimally invasive heart surgery or open-heart surgery. Mitral valve repair surgery should generally be performed at a medical center with staff that has experience in performing mitral valve repair surgery and that has conducted high volumes of mitral valve repair surgeries. If a patient has mitral valve disease, he or she may eventually need mitral valve repair or mitral valve replacement surgery to treat the condition.

Doctors may often recommend mitral valve repair if possible, as it preserves the mitral valve and may preserve heart function. Having the mitral valve repaired may also help avoid complications that can occur with mitral valve replacement, such as the risk of blood clots with mechanical valves and the risk of biological tissue valves failing over time.

During the procedure, the patient will receive anesthetics, and be unconscious. The patient will usually be connected to a heart-lung bypass machine, which keeps blood moving through the body during the procedure.

Mitral valve surgery generally may be performed with open-heart surgery, which involves a cut (incision) in the chest.

In some cases, mitral valve repair surgery may be performed with minimally invasive heart surgery, in which surgeons perform the procedure through small incisions in the chest. Minimally invasive heart surgery includes surgery performed using long instruments inserted through one or more small incisions in the chest (thoracoscopic surgery), surgery performed through a small incision in the chest, or surgery performed by a surgeon using robotic arms (robot-assisted heart surgery). In robot-assisted heart surgery, a surgeon sits at a remote console, viewing the heart in a magnified high-definition 3-D view on a video monitor. The surgeon uses robotic arms to duplicate specific maneuvers used in open-heart surgeries. These procedures may be available at certain medical centers. Minimally invasive heart surgery may involve a shorter hospital stay, quicker recovery and less pain than open-heart surgery. Minimally invasive heart surgery generally should be performed at medical centers with a medical team experienced in performing these types of procedures.

Mitral valve repair surgery may include patching holes in a valve, reconnecting valve leaflets, removing excess valve tissue so that the leaflets can close tightly, replacing cords that support the valve to repair the structural support, and separating valve leaflets that have fused. Surgeons may tighten or reinforce the ring around the valve (annulus) in a procedure called an annuloplasty. Doctors may perform certain mitral valve repair procedures using a long, thin tube (catheter) and clips or other devices. In one catheter procedure, doctors insert a catheter with a clip attached in an artery in the groin and guide it to the mitral valve. Doctors use the clip to reshape the mitral valve. People who have severe symptoms of mitral valve regurgitation and who are not candidates for surgery or who have high surgical risk may be considered for this procedure. Doctors may also use a catheter procedure to insert a device to plug leaks in a leaking replacement mitral valve that previously has been implanted to replace the mitral valve. A mitral valve with a narrowed opening may also be treated with a catheter procedure called a balloon valvuloplasty. In this procedure, a doctor inserts a catheter with a balloon on the tip into an artery in the patient's arm or groin and guides it to the mitral valve. A doctor then inflates the balloon, which expands the opening of the mitral valve. The balloon is then deflated, and the catheter and balloon are removed.

If a diseased mitral valve cannot be repaired, the doctor may recommend mitral valve replacement. In this procedure, the doctor removes the mitral valve and replaces it with a mechanical valve or a valve made from cow, pig or human heart tissue (biological tissue valve). Biological tissue valves often eventually need to be replaced, as they degenerate over time. If a mechanical valve is used, the patient will need to take blood-thinning medications to prevent blood clots. When possible, doctors may preserve the cords supporting the valve during the procedure, to preserve as much of the heart's function as possible. In some cases, a catheter procedure may be conducted to insert a replacement valve in a biological tissue valve in the heart that is no longer working properly. Some doctors are also studying using catheter procedures to replace a mitral valve that is no longer working properly, and some medical centers may offer this procedure as part of clinical trials for people with severe mitral valve disease who are not candidates for surgery.

In recovery, a patient will generally spend a day or more in the intensive care unit (ICU) and be given fluids, nutrition and medications through intravenous (IV) lines. Other tubes will drain urine from the bladder and drain fluid and blood from the heart and chest. The patient also may be given oxygen. After the ICU, the patient will be moved to a regular hospital room for several days. The time spent in the ICU and hospital can vary, depending on the condition and the type of surgery. The treatment team may monitor the patient's condition and watch for signs of infection in incision sites. As part of the monitoring, the team may check blood pressure, breathing and heart rate. The treatment team will also help manage pain experienced after surgery. The patent may be encouraged or instructed to walk regularly to gradually increase activity and to do breathing exercises as recovery progresses. The doctor may give instructions to follow during recovery, such as watching for signs of infection in the incisions, properly caring for incisions, taking medications, and managing pain and other side effects after surgery.

After mitral valve repair or mitral valve replacement surgery, the patient may be able to return to daily activities, such as working, driving and exercise. Follow up care may include taking certain medications and monitoring of the condition. Sometimes, additional changes to lifestyle may be required, including more frequent physical activity, a more healthy diet, improved stress management and reductions in tobacco use. Other changes may include cardiac rehabilitation a program of education and exercise designed to help improve health and recovery after heart surgery.

### Transcatheter Aortic Valve Replacement or Implantation

Aortic valve stenosis, or aortic stenosis, occurs when the heart's aortic valve narrows. This narrowing prevents the valve from opening fully, which reduces or blocks blood flow from the heart into the main artery to the body (aorta) and onward to the rest of the body. When the blood flow through the aortic valve is reduced or blocked, the heart needs to work harder to pump blood to the body. Eventually, this extra work limits the amount of blood the heart can pump, and this can cause symptoms as well as possibly weaken the heart muscle. The treatment depends on the severity of the condition and may require surgery to repair or replace the valve. Left untreated, aortic valve stenosis can lead to serious heart problems.

Current methods of treatment for diagnosed aortic valve stenosis are limited to invasive surgical techniques. After initial diagnosis of aortic valve stenosis, a transcatheter aortic valve replacement can be surgically conducted. While such surgical treatment can save lives and improve quality of life for those suffering aortic valve stenosis, dangers beyond those of the surgery itself still exist for the patient due to possible post-surgery complications (for example, neurological injuries, bleeding, or stroke) as well as device-related complications (for example, thrombosis, leakage, or failure).

Aortic valve stenosis is not the only condition for which enzymatic degradation of structural proteins is a hallmark. Other conditions in which structural protein degradation appears to play a key role include aneurisms and Marfan syndrome. For those afflicted, such conditions lead to, at the very least, a lowered quality of life and often, premature death.

Aortic valve stenosis—or aortic stenosis occurs—when the aortic valve narrows. This narrowing prevents the valve from opening fully, which obstructs blood flow from the heart into the aorta and onward to the rest of the body. Aortic stenosis can cause chest pain, fainting, fatigue, leg swelling and shortness of breath. It may also lead to heart failure and sudden cardiac death. Severe aortic stenosis is a condition that prevents blood from flowing easily throughout your body. The heart may need to work harder to pump blood throughout the body, and many times, it cannot do so effectively. When that happens, some people may notice uncomfortable symptoms such as shortness of breath and fatigue as the heart becomes weaker. Without aortic valve replacement, only a few people with the disease survive past 5 years. The good news is, there is hope and a less invasive treatment option available for severe aortic stenosis. Medication cannot stop or cure the disease, it can only treat the symptoms. Valve replacement is the only effective treatment option. At least 40% (and perhaps up to 60%) of patients with severe aortic stenosis do not receive valve replacement. Open heart surgery is not the only option for treating severe aortic stenosis.

Transcatheter Aortic Valve Replacement (TAVR) is a minimally invasive procedure to replace a narrowed aortic valve that fails to open properly (aortic valve stenosis). TAVR may be an option for people who are considered at intermediate or high risk of complications from surgical aortic valve replacement. TAVR may also be indicated in certain people who can't undergo open-heart surgery. The decision to treat aortic stenosis with TAVR is made after consultation with a multidisciplinary group of medical and surgical heart specialists who together determine the best treatment option for each individual. TAVR can relieve the signs and symptoms of aortic valve stenosis and may improve survival in people who can't undergo surgery or have a high risk of surgical complications.

TAVR may be an option for treatment of aortic stenosis that causes signs and symptoms. For instance, people who are candidates for TAVR may include those who are considered at intermediate or high risk of complications from surgical aortic valve replacement. Conditions that may increase the risk of surgical aortic valve replacement include lung disease or kidney disease which increase risk of complications during surgical aortic valve replacement. TAVR may also be an option for patients with an existing biological tissue valve that was previously inserted to replace the aortic valve, but it isn't functioning well anymore.

TAVR carries a risk of complications, which may include: Bleeding, Blood vessel complications, Problems with the replacement valve, such as the valve slipping out of place or leaking, Stroke, Heart rhythm abnormalities (arrhythmias), Kidney disease, Heart attack, Infection, or Death.

As a procedure, TAVR involves replacing a damaged aortic valve with one made from cow or pig heart tissue, also called a biological tissue valve.

A patient may receive general anesthesia before the TAVR procedure. A treatment team member will give medication through an intravenous line to prevent blood clots. The treatment team will monitor the patient's heart function and rhythm, and watch for changes in heart function that may occur. Changes in function can be managed with treatments as needed during the procedure. During TAVR, doctors may access the patient's heart through a blood vessel in the leg. Alternatively, the doctors may conduct the procedure through a tiny incision in the chest, and access the heart through a large artery or through the tip of the bottom left chamber of the heart (left ventricle). Doctors may sometimes use other approaches to access the heart. In TAVR, a hollow tube (catheter) is inserted through the access point. The doctor uses advanced imaging techniques to guide the catheter through the blood vessels, to the heart and into the aortic valve. Once it is precisely positioned, a balloon is expanded to press the replacement valve into place where the native aortic valve, prior to surgical removal, would be. Some valves can expand without the use of a balloon. When the doctor is certain the valve is securely in place, the catheter is withdrawn from the blood vessel or from the incision in the chest.

Current recovery from TAVR may include a night in the intensive care unit for monitoring after the procedure and about two to five days recovering in the hospital. The patient will need to take blood-thinning medications for a period of time to prevent blood clots. Additionally, certain medications may be prospectively required for certain dental procedures to prevent certain infections, as patients are at higher risk of certain infections with a replacement heart valve.

TAVR can improve the lives of people with aortic stenosis who can't have surgery or for whom surgery is too risky. In these people, TAVR can reduce the risk of death. TAVR may also relieve the signs and symptoms of aortic valve stenosis and improve overall health. Some studies have found that TAVR has similar mortality rates as heart valve surgery in people with aortic stenosis who have an intermediate or high risk of complications from open-heart surgery.

Transcatheter aortic valve replacement (TAVR) is a procedure designed to replace a diseased aortic valve. For people with the severe disease of the aortic valve, surgery to replace the valve is often the only treatment that offers substantial relief. However, aortic valve surgery exposes patients to significant risks, and sometimes those risks prohibit surgery. Transcatheter aortic valve implantation (TAVI) was developed in the attempt to devise a less invasive, less risky approach to replacing diseased aortic valves.

This procedure used to only be available for people who were too weak to undergo open heart surgery. But now, TAVI is available to more people depending on their risk for open heart surgery. TAVI is different from open heart surgery in that it uses a less invasive approach to treat a diseased aortic valve.

In TAVI, an artificial aortic valve is implanted by means of a sophisticated catheterization procedure. While TAVI is still considered a new procedure, it is approved in the United States and the European Union for the treatment of certain high-risk patients with severe aortic stenosis. In Europe, it is also approved for treating some people with severe aortic regurgitation.

In aortic stenosis, the aortic valve becomes partially obstructed, which forces the heart to work much harder to pump blood to the body. In aortic regurgitation, the aortic valve fails to close completely, allowing blood to flow back into the heart when the valve is supposed to be closed. Eventually, either of these aortic valve conditions can progress to heart failure, causing edema (swelling), dyspnea, and (often) death. While symptoms of aortic valve disease can be managed for a time using medical therapy for heart failure, no medicine can relieve a mechanical problem with the aortic valve. The only really effective treatment is to surgically replace the diseased aortic valve with an artificial valve. Unfortunately, the standard method of aortic valve replacement requires a major open-heart surgical procedure, and, especially in the elderly patients who most typically develop aortic stenosis, it is a procedure that carries significant risk. The TAVI procedure has been developed as a potentially lower-risk approach to replacing the aortic valve.

Several TAVI systems have been developed by various medical device companies, and while each device has its unique features, all of them work similarly. The artificial valve is attached to a collapsed wire frame, which is in turn attached to a catheter. The catheter is inserted into a blood vessel (usually, the femoral artery near the groin), and is advanced to the area of the aortic valve. When in position, the wire frame is expanded by blowing up a balloon. This allows the artificial valve to open up and attach itself, and to begin functioning. Balloons suitable for use in expanding the wire frame and for delivering therapeutic agents to the implantation site are described below.

The doctor will determine the best approach for replacing the valve, but the most common technique involves a small incision made in the leg. This is called the transfemoral approach. The doctor will perform the procedure at a hospital. Depending on the patient's health, the best type of anesthesia is determined. The patient may be fully asleep or may be awake but given medication to relax and block pain. The patient's heart will continue to beat during the procedure. This is quite different to open heart surgery, in which the patient's heart is stopped, and the patient placed on a heart and lung blood machine.

In step 1 of the procedure, a small incision is made in the patient's upper leg. This is where the surgeon will insert a short, hollow tube called a sheath into the patient's femoral artery. In step 2, a new valve is placed on a delivery system (or tube). The new valve is compressed to make it small enough to fit through the sheath. In step 3, the delivery system carrying the valve is pushed up to the patient's aortic valve. Once it reaches the valve, the new valve pushes aside the leaflets of the diseased valve. The patient's existing valve holds the new valve in place. In step 4, the new valve will open and close as a normal aortic valve should. The surgeon will assure that the new valve is working properly before closing the incision.

Other ways to perform the TAVI procedure include a transapical approach (through an incision in the chest between the ribs) or a transaortic approach (through an incision in the upper chest.

Early studies with TAVI were limited to patients with severe aortic stenosis who were deemed too sick to have the open-heart surgery necessary for “standard” aortic valve replacement. In these very sick patients, those who were randomized to receive TAVI had a significantly reduced mortality rate and significantly improved symptoms after one year, compared to those who received medical therapy alone. However, patients randomized to TAVI had a 5% incidence of major stroke, compared to only 1% in medically treated patients. TAVI-related strokes are embolic strokes. A later study compared TAVI to standard aortic valve replacement in 690 patients with severe aortic stenosis. The mortality rates, stroke rates, and symptom improvement were similar at one year in both groups. Those treated with TAVI had more major complications to the blood vessels, and those treated with open-heart surgery had more bleeding complications and more post-operative atrial fibrillation.

While TAVI is much less invasive than open heart surgery, it still carries significant risks. The two most frequent and serious risks are serious damage to the major blood vessels, and stroke. Both of these complications are due to often-unavoidable trauma caused by inserting the sizable and relatively rigid valve mechanism into an aorta that is often diseased. As a result of such complications, the risk of death with TAVI is around 6% within 30 days of the procedure. Recent evidence suggests there is a steep “learning curve” associated with performing the TAVI procedure. In particular, the risk of serious complications seems to be highest during the first 30 TAVI procedures a doctor performs. The companies that are developing TAVI devices continue to work on improving the technology, in an attempt to reduce the risks associated with their use. For the present, however, the risks remain substantial.

At its present state of development, TAVI is reserved for people who have significant aortic stenosis (or in some regions, aortic regurgitation), in whom the risk of standard aortic valve replacement surgery is deemed to be extremely high. In particular, current guidelines recommend TAVI in patients whose estimated risk of surgical death or serious irreversible complications is 50% or higher. In people whose surgical risk is considered low, then standard valve replacement surgery is recommended. In those whose surgical risk is intermediate, the decision regarding surgery or TAVI is made on a case by case basis.

### Tumors and Tumor Beds

A tumor is an abnormal mass of tissue that results when cells divide more than they should or do not die when they should. Tumors may be benign (not cancer), or malignant (cancer). An abnormal mass of tissue that usually does not contain cysts or liquid areas is referred to as a solid tumor. Different types of solid tumors are named for the type of cells that form them. Examples of solid tumors are sarcomas, carcinomas, and lymphomas.

Hepatic (or liver) tumors include a diverse group of masses that include malignant and benign subtypes. Their presentation can vary from localizing signs/symptoms, such as jaundice and right upper quadrant pain, to vague signs/symptoms, such as fatigue, weight loss, and anorexia. Many hepatic tumors are discovered incidentally on medical imaging studies. Additionally, the liver is a common site for metastasis, and metastases to the liver are significantly more common than primary liver tumors.

Common malignant liver tumors include the following: hepatocellular carcinoma (most common primary malignant tumor), intrahepatic cholangiocarcinoma, hepatoblastoma, angiosarcoma, metastases (most common malignant liver tumors). Common benign liver tumors include the following: hemangioma (most common benign tumor), hepatic adenoma, focal nodular hyperplasia, and hepatocellular carcinoma (HCC).

HCC is the most common primary malignant liver tumor in adults. It occurs predominantly in patients with underlying chronic liver disease and cirrhosis, thus, there is a strong association between the development of HCC and infection with HBV and hepatitis C virus (HCV). Typically, the pattern of HCC spread is local expansion, but it can also metastasize via the hematogenous route and it can be multifocal. In general, these tumors are discovered either during routine screening in cirrhotic patients or when the lesions are symptomatic owing to their size or location. Selected patients with HCC may be surgical candidates for curative-intent treatment. Liver resection is the preferred treatment in noncirrhotic patients with tumors smaller than 5 cm However, in cirrhotic patients, the ability to tolerate a formal liver resection is limited by the degree of cirrhosis. Liver transplantation can be considered in patients with established cirrhosis. Patients with advanced disease or those who are otherwise not candidates for liver resection or transplantation may be offered chemotherapy, radiotherapy, targeted therapy, embolization, chemoembolization, or local ablative treatments.

Cholangiocarcinoma, or bile duct carcinoma, is a relatively rare liver tumor that is primarily classified as an adenocarcinoma. In general, cholangiocarcinomas have a very aggressive biology and are usually metastatic at the time of presentation. These tumors are classified into three major subtypes on the basis of their location, as follows: perihilar (also known as Klatskin tumors) originate from the extrahepatic biliary tree close to the hepatic duct bifurcation, intrahepatic originating from intrahepatic bile ducts, and distal (extrahepatic) originating close to the ampulla of Vater. Complete surgical resection of cholangiocarcinoma is the only option for potential cure. Liver transplantation may be considered for select patients with proximal tumors who are not candidates for surgical resection due to underlying cirrhosis. Radiation therapy, usually in conjunction with chemotherapy, may be used in an attempt to make borderline resectable tumors into resectable lesions.

For most tumor types, liver metastasis is considered stage IV disease in the American Joint Committee on Cancer (AJCC) Staging System. However, the management of these tumors has evolved significantly over the past decade and is usually dictated by the type of primary tumor, the size and number of lesions, and the patient's clinical condition. Local ablative therapies such as microwave ablation (MWA), radiofrequency ablation (RFA), transarterial chemoembolization (TACE), and irreversible electroporation (IRE) continue to evolve and have important roles in the management of unresectable tumors.

Hepatic hemangiomas (or, hepatic venous malformations, hepatic cavernous hemangiomas) are the most common benign tumors affecting the liver. Their etiology remains unknown, but these tumors are mesenchymal in origin, usually solitary, have a female preponderance (5:1), and are associated with some genetic syndromes (e.g., Kassabach-Merritt syndrome, Osler-Weber-Rendu disease). Most of these lesions are asymptomatic and discovered incidentally on imaging studies. Due to their benign nature and no known malignant potential, hepatic hemangiomas generally do not require treatment. For large, symptomatic hemangiomas that affect the patient's quality of life or when the diagnosis is in doubt, surgery may be considered.

Focal nodular hyperplasia (FNH) is the second most common benign tumor of the liver after hepatic hemangiomas. It is generally found incidentally and affects women more often than men. Although patients are rarely symptomatic, FNH can cause abdominal pain and vague upper gastrointestinal symptoms. FNH is not considered a premalignant condition; once a correct diagnosis has been made, there is no indication for surgery in most cases, and treatment includes conservative clinical follow-up in asymptomatic patients. Surgery is generally reserved only for large symptomatic FNH or when the diagnosis is in doubt.

Hepatic adenoma, or hepatocellular adenoma, is a rare, usually benign tumor that occurs mostly in women of childbearing age; it is strongly associated with the use of oral contraceptive pills (OCPs). In men, hepatic adenoma is associated with the use of anabolic steroids. Once diagnosed, patients with hepatic adenoma should discontinue OCPs or any anabolic steroids. Ruptured adenomas may require emergent intervention for hemorrhagic control. Surgical resection is classically indicated for symptomatic hepatic adenomas or those larger than 5 cm.

Simple hepatic cysts are benign and have no malignant potential. These large simple cysts appear to affect women slightly more than men. Simple hepatic cysts may be asymptomatic, or they can produce right upper quadrant or flank pain; rarely, the tumors can cause biliary obstruction or intracystic hemorrhage. Surgical intervention, usually cyst unroofing (or, open surgical cyst fenestration, marsupialization), is offered in symptomatic cases; needle aspiration and sclerotherapy are also options, but these are associated with higher rates of recurrence. Complete cyst excision or hepatectomy may be performed when the diagnosis is in doubt.

Surgery remains the mainstay of treatment for most liver tumors. Ablation refers to the local destruction of tumors without surgical resection. Most of these techniques are used in tumors that are otherwise unresectable. Multiple different techniques have evolved over the past several years, including the following: microwave ablation, radiofrequency ablation, cryoablation, chemical (ethanol/alcohol) ablation, or irreversible electroporation. Although each technique offers advantages and disadvantages over the others, all require the placement of probes directly into the tumor. Percutaneous and surgical approaches are acceptable for these ablative techniques.

Regional therapies are used in the setting of widespread intrahepatic tumor burden that is not amenable to surgical resection. Although multiple techniques have gained significance in the past several years, regional therapies are usually delivered intraarterially to the liver, with the advantage of conveying high dosages with minimal extrahepatic toxicity. Common techniques for regional therapies of the liver include the following: transarterial chemoembolization (TACE)—chemotherapy is delivered in combination with embolization of the tumor blood supply; transarterial bland embolization (TAE)—embolization of the tumoral blood supply is administered without chemotherapy, usually to occlude larger vessels; radioembolization/transarterial radioembolization (TARE)—radiolabeled microspheres/radiation beads (yttrium-90 [⁹⁰Y]) are delivered intraarterially to tumor cells; (transcatheter arterial) intraarterial chemoinfusion (TACI)—high-dose chemotherapy is delivered directly to the liver utilizing an intraarterial catheter with a subcutaneous pump for administration over multiple cycles. These techniques can still have regional and/or hepatic side effects, thereby making the selection of the appropriate technique complex.

Prostate cancer is a tumor of the prostate, a gland that is located in front of the rectum, above the base of the penis and below the bladder, where urine is stored. The prostate gland surrounds the first part of the urethra, the tube that connects the bladder with the tip of the penis and carries urine and other fluids out of the body. The prostate helps make the milky fluid called semen that carries sperm out of the body when a man ejaculates. Prostate cancer is typically a slow growing cancer that shows few symptoms, but some types may be aggressive and spread rapidly.

Prostate cancer is the most common form of cancer in American men. It is most prevalent in men over age 65 and fairly common in men 50-64 years old. However, prostate cancer can occur in men younger than 50. The incidence of diagnosed prostate cancer among American men has increased dramatically since 1990 because of the use of a screening blood test called prostate-specific antigen (PSA). More recently, men below the age of 65 years have shown an increased incidence of this disease.

Risk factors of prostate cancer include: age, race (especially men of African-American descent), obesity, family history of prostate cancer, diet high in fats from red meat, and history of sexually transmitted disease (STD).

Prostate cancer shows few symptoms until its advanced stages. These symptoms include: blood in urine or semen, lower back, pelvic or hip pain, urination issues, erectile dysfunction.

In some cases of early prostate cancer, there are no symptoms and the cancer is often discovered through routine screening with PSA blood test and/or digital rectal examination of the prostate.

There are many treatment options for prostate cancer that is confined to the prostate gland. Each option should be considered carefully, balancing the advantages against the disadvantages as they relate to the individual's age, overall health, the aggressiveness and/or stage of the cancer and the patient's personal preferences.

Conventional treatment options include surgery (radical prostatectomy), where an incision is made in the lower abdomen or through the perineum (between the anus and the scrotum), and the prostate is removed. In incomplete surgery, in which the entire tumor cannot be removed, radiation therapy may follow surgery. The patient is required to keep a urinary catheter in place for a number of weeks after the procedure. Possible side effects of surgery can include incontinence (inability to control urination) and impotence (inability to achieve erection).

External beam therapy (EBT) is a method for delivering a beam of high-energy x-rays or proton beams to the location of the tumor. The radiation beam is generated outside the patient (usually by a linear accelerator for photon/x-ray and a cyclotron or synchrotron for proton beam) and is targeted at the tumor site. These radiation beams can destroy the cancer cells, and conformal treatment plans allow the surrounding normal tissues to be spared.

In active surveillance, no treatment is administered, but careful observation and medical monitoring is conducted.

In nerve-sparing radical prostatectomy, the prostate gland is removed without severing the critical nearby nerves that send signals between the brain and penis to allow normal sexual functioning. A skilled and experienced surgeon may be able to preserve sexual function for some patients by successfully using this procedure.

Conformal or intensity modulated external beam radiation therapy is an external beam radiation therapy that uses high energy photons which can kill cancer cells. Conformal or intensity modulated radiation therapy techniques use advanced technology to tailor the radiation therapy to an individual's body structures. Relying on computerized three-dimensional images of the prostate, bladder and rectum, the x-ray radiation beam is shaped to conform to the prostate gland and sometimes to nearby lymph nodes. In this way, less radiation reaches the surrounding normal tissues. There are two levels of conformal radiation therapy: 3-D conformal radiation therapy and intensity modulated radiation therapy (IMRT). Both allow for increased doses to the tumor while protecting the normal surrounding organs. IMRT is considered the more highly focused of the two. Treatments are typically given daily (Monday through Friday) for four to nine weeks.

Stereotactic Body Radiation Therapy (SBRT) is another form of conformal external beam radiation therapy, uses photon or x-ray therapy at a much larger dose per treatment to treat the prostate over one to two weeks with four to five treatments. SBRT requires higher precision and requires special equipment. Not all patients are candidates for SBRT.

Proton beam therapy is a type of conformal radiation therapy that bombards the diseased tissue with proton particles instead of x-rays (photons). With a multiple beam setup, the high-dose area around the tumor is similar between protons and x-rays with IMRT. There is, however, less low- and moderate-dose radiation delivered to surrounding normal tissues (bowels, bladder, bone, soft tissues) with protons. Proton beam therapy is more costly compared to other radiation treatments, and the potential clinical benefits are currently the subject of ongoing investigation.

Image-guided radiation therapy (IGRT) is a therapy in which 3-D conformal, IMRT, SBRT or proton therapy is used, and daily image guidance is used to improve the setup due to organ movement. Since the prostate position varies day-to-day depending on bladder and rectal filling, the prostate position must be localized and verified prior to each treatment. In one method, several fiducial markers (tiny pieces of biologically inert material such as gold or carbon) are placed in the prostate gland before the simulation. Digital x-ray images are taken which localize the metallic markers to check the position of the prostate on a daily basis just before the treatment and make appropriate adjustments and alignment of the prostate within the planned high-dose radiation treatment field. Another method involves using ultrasound to localize the prostate before each treatment. The patient is asked to keep his bladder full as much as possible in order to produce a good ultrasound image and to displace the bladder mucosa out of the radiation treatment field. Other methods involve the use of low-dose computed tomography (CT) scanning and/or MRI scanning of the prostate in the treatment couch prior to each treatment to verify prostate position.

Cryotherapy is a procedure that uses extremely low temperatures (−190° C.) to freeze and destroy cancer cells. This technique was developed as an alternative to surgery for patients who have recurrent cancer in the prostate after radiation treatments.

Brachytherapy is radiation treatment delivered to the prostate via the placement of radioactive materials inside the prostate. There are two forms of brachytherapy, including low-dose rate (LDR) and high-dose rate (HDR). Low-dose rate (LDR) brachytherapy or permanent seed implant treatment involves about one hundred small radioactive seeds inserted into the prostate gland through hollow needles under ultrasound or MRI guidance. These radioactive seeds deliver radiation continuously over a period of several weeks to months then become inactive. These metal seeds remain in the prostate forever. While the implant technique has been around for decades, recent advances in imaging technology have made it more effective. Prior to the implant, imaging such as CT, MRI or ultrasound is performed in order to plan the procedure. The implant procedure is done under conscious sedation or local/regional anesthesia. During the implant procedure, ultrasound (or sometimes MRI) is used to see the prostate gland better. Using needles, physicians can insert the seeds into the prostate more carefully transperineally (through the skin behind the testicle and in front of the anus). This is an outpatient procedure, and the patient may be required to keep a urinary catheter in place for about a week. Long-term results are available for up to 15 to 20 years at some institutions. These results show that in experienced centers, ultrasound-guided radioactive seed implantation is highly effective in controlling prostate cancer and has essentially the same result as surgery or external beam radiation for appropriately selected prostate cancer patients. High Dose Rate (HDR) Brachytherapy was developed to supplement external beam therapy to treat patients with high risk prostate cancer. Patients receive about five weeks of external beam radiation therapy, followed by one to three HDR brachytherapy sessions. In this treatment, the radiation is delivered into the prostate via radioactive isotopes (often, Iridium-192) temporarily. This procedure is done as an in-patient procedure. First, about 12 to 18 hollow catheters are inserted into the prostate transperineally using ultrasound and x-ray guidance while patient is under general anesthesia. Then, a CT scan and treatment planning are done to determine location and duration of the placement of the Iridium-192 source. When the patient receives treatment, these catheters are connected to the HDR machine, which controls the delivery of the Iridium-192 radioactive source to the specific areas in each of these catheters. The treatment often lasts about 10 to 20 minutes per session, and the patient usually receives three to four sessions over a two-day period. At the end of the last session, the catheters are removed from the patient, and he is released from the hospital. While the catheters are in the prostate, the patient is required to be bed-ridden and hospitalized during that two-day period. The patient does not have permanent radioactive materials when he leaves the hospital and may be required to keep a urinary catheter in place for about a week.

Radium 223 treatment utilizes an isotope of the metal radium that is used to treat prostate cancers that have spread to the bones. Because of its chemical similarity to calcium, radium is absorbed by bone cells. Because cancer cells are more active than normal bone cells, they are more likely to absorb the radium 223. Once the radium is in the bones, it releases radiation within a very small area to kill the nearby cancer cells while sparing the healthy bone cells surrounding the cancer. Radium 223 is effective at controlling advanced prostate cancer and reducing pain in more than one area of the bone because it travels throughout the body. The injection takes up to a minute and is typically repeated every four weeks for up to six or more total treatments. Treatment is performed on an outpatient basis, so you may return home afterwards. The side effects of radium 223 include diarrhea, anemia and pain in the areas of the tumor where the radium is working. Men who receive radium treatment shouldn't father children for at least six months because radium may cause sperm damage.

Conditions characterized by enzymatic degradation of structural proteins include Marfan syndrome, aneurysm, and supravalvular aortic stenosis. For those afflicted, such conditions lead to, at the very least, a lowered quality of life and often, premature death. Modifying protein degradation in tumors and adjacent tissues may be a valuable therapeutic approach to treatment of tumors. Global inhibition of protein degradation systems may reverse, halt, or slow tumor progression, and the ability to modulate these processes more selectively offers huge potential for the developing novel therapeutics, while avoiding negative side effects

### Pelvic Organ Prolapse

Pelvic organ prolapse is a type of pelvic floor disorder. The most common pelvic floor disorders are: urinary incontinence (leaking of urine), fecal incontinence (leaking of stool), and pelvic organ prolapse (weakening of the muscles and tissues supporting the organs in the pelvis). Pelvic organ prolapse happens when the muscles and tissues supporting the pelvic organs (the uterus, bladder, or rectum) become weak or loose. This type of hernia allows one or more of the pelvic organs to drop or press into or out of the vagina.

Normally, a hammock of muscles, ligaments, and fibers attach to the bony anatomy of the pelvis and support the pelvic organs. The pelvic organs include the bladder, uterus and cervix, vagina, and rectum, which is part of the bowel. A prolapse happens when the pelvis muscles and tissues can no longer support these organs because the muscles and tissues are weak or damaged. This causes one or more pelvic organs to drop or press into or out of the vagina. There are two types of pelvic organ prolapse, asymptomatic and symptomatic. Asymptomatic prolapse means that while the hernia has taken place, nothing extends beyond the vaginal opening. Symptomatic prolapse refers to when there is tissue that is protruding past the vaginal opening.

The different types of pelvic organ prolapse depend on the pelvic organ affected. The most common types include: dropped bladder (called cystocele), which happens when the bladder drops into or out of the vagina; rectocele, which happens when the rectum bulges into or out of the vagina; and dropped uterus (uterine prolapse), which happens when the uterus bulges into or out of the vagina. Uterine prolapse is sometimes associated with small bowel prolapse (called enterocele), where part of the small intestine, or small bowel, bulges into the vagina. Although it is rare, pelvic organ prolapse can also happen after a hysterectomy. Any part of the vaginal wall may drop, causing a bulge into or out of the vagina.

Pelvic floor disorders (urinary incontinence, fecal incontinence, and pelvic organ prolapse) affect one in five women in the United States. Pelvic organ prolapse is less common than urinary or fecal incontinence but affects almost 3% of U.S. women. Pelvic organ prolapse happens more often in older women and in white and Hispanic women than in younger women or women of other racial and ethnic groups. Some women develop more than one pelvic floor disorder, such as pelvic organ prolapse with urinary incontinence.

There are multiple degrees of severity of prolapse: in a very mild prolapse the organs are still fairly well supported by the pelvic floor; in a moderate prolapse, the pelvic floor organs have begun to fall, but are still contained inside the vagina; in a severe prolapse, the pelvic floor organs have fallen to, or beyond the opening of the vagina; and in a very severe prolapse, the pelvic floor organs have fallen completely through the vaginal opening.

The pressure from prolapse can cause a bulge in the vagina that can sometimes be felt or seen. Women with pelvic organ prolapse may feel uncomfortable pressure during physical activity or sex. Other symptoms of pelvic organ prolapse include: seeing or feeling a bulge or “something coming out” of the vagina, a feeling of pressure, discomfort, aching, or fullness in the pelvis, pelvic pressure that gets worse with standing or coughing or as the day goes on, leaking urine (incontinence) or problems having a bowel movement, or problems inserting tampons. Some women say that their symptoms are worse at certain times of the day, during physical activity, or after standing for a long time.

The most common risk factors for pelvic organ prolapse are: vaginal childbirth, which can stretch and strain the pelvic floor (multiple vaginal childbirths raise risk for pelvic organ prolapse later in life, but prolapse can happen even if the patient never had children or had children with a cesarean (C-section) delivery); long-term pressure on the abdomen, including pressure from obesity, chronic coughing, or straining often during bowel movements; giving birth to a baby weighing more than 812 pounds; aging (about 37% of women with pelvic floor disorders are 60 to 79 years of age, and about half are 80 or older); hormonal changes during menopause (loss of estrogen during and after menopause can raise risk for pelvic organ prolapse); and family history.

Treatment for pelvic organ prolapse depends on the type of prolapse, symptoms, age, other health problems, and whether the patient is sexually active. A first non-surgical treatment option is a pessary. A pessary is a removable device inserted into the vagina to support the pelvic organs. Pessaries come in many different shapes and sizes and certain types of pessaries can treat both pelvic organ prolapse and urinary incontinence. A second non-surgical treatment option is pelvic floor muscle therapy. This type of treatment includes pelvic floor exercises to help strengthen the pelvic floor muscles. Pelvic floor muscle exercises can also help women who have pelvic organ prolapse as well as urinary incontinence. A third non-surgical treatment option may include changing eating habits to eat more foods with fiber. Fiber helps prevent constipation and straining during bowel movements.

While reconstructive surgery for pelvic organ prolapse is an option, there is a 30% recurrence rate for women choosing this route. Prolapse repairs can be done transvaginally, abdominally, laparoscopically, and/or robotically (when a scope is placed through the belly button). Ultimately, the purpose of the surgery is to correct the anatomy as well as provide better bowel, bladder, and vaginal function.

Surgical options include cystocele repair, which repairs a prolapsed bladder or urethra (urethrocele); hysterectomy, which is a complete removal of the uterus; rectocele repair, which repairs the fallen rectum or small bowel (enterocele); and vaginal vault suspension, which is a laparoscopic procedure to repair the vaginal wall.

Some surgical options may include adding support to the uterus or vagina. For example, the surgeon may use the patient's own body tissue or synthetic mesh to help repair the prolapse and build pelvic floor support. This type of surgery is recommended for sexually active women with serious prolapse of the vagina or uterus. Surgery for prolapse can be done with or without mesh and either through the vagina or abdomen. While abdominal repairs are believed to have higher success rates, the increase in morbidity makes this also one of the riskier options. Vaginal grafts (made of synthetic and biologic materials) are also being explored as a long-term solution to pelvic organ prolapse.

An alternative surgical option to treat prolapse, called colpocleisis or vaginal obliteration, surgically closes the vaginal opening so that it no longer prolapses. This type of transvaginal surgery has nearly a 100% success rate and is usually reserved for elderly patients with multiple medical problems. After these surgeries, vaginal intercourse is no longer physically possible.

### Pentagalloyl Glucose (PGG)

Certain risks associated with laxity of tendons or ligaments, particularly post-surgery laxity of tendons or ligaments, or with surgery to soft tissue, particularly surgical tendon repair, or with treatment of peripheral vascular disease or congestive heart failure or mitral valve treatment or SUI or pelvic organ prolapse can be ameliorated by delivery of pentagalloyl glucose (PGG), e.g., 1,2,3,4,6-pentagalloyl glucose. The PGG can be delivered to the surgical site or to the implantation site or to the tendon or soft tissue or vasculature or cardiac tissue (e.g., heart muscle, wall of the heart, the chambers of the heart, the mitral valve), or to a blood vessel or other body lumen, or to pelvic organs or supporting tissue (e.g., ligaments and tendons). Certain risks associated with surgical treatment of SUI or pelvic organ prolapse can be mitigated by delivery of pentagalloyl glucose (PGG), e.g., 1,2,3,4,6-pentagalloyl glucose, e.g. to the site of implantation of a supportive device, e.g., a urogynecologic surgical mesh, or the site of reconstructive surgery.

In an embodiment, PGG may be delivered to cardiac tissue (e.g., heart muscle, wall of the heart, the chambers of the heart) for treatment of congestive heart failure. Without being limited by theory, the delivery of PGG to the cardiac tissue may stabilize the heart muscle by cross-linking, at least transiently, the elastin proteins within the extracellular matrix of the cardiac connective tissue. Treatment of the cardiac tissue with an elastin-stabilizing compound, such as PGG, may increase the mechanical integrity of the cardiac tissue. Treatment with PGG may prevent, inhibit, and/or slow the progression of congestive heart failure by strengthening the associated cardiac tissue. In some instances, treatment with PGG may facilitate natural healing of damaged cardiac tissue by mechanically stabilizing the cardiac tissue. In some implementations, treatment with PGG may be used prior to, after, and/or concurrently with other interventional treatment, such as surgical intervention.

PGG may be delivered to the cardiac tissue by devices as described herein, e.g., by an intravascular catheter through a vascular access hole, e.g., in the femoral artery. In some embodiments, PGG, particularly a high purity PGG as disclosed herein, may be suitable for direct injection into the bloodstream or into cardiac tissue for treatment of congestive heart failure. PGG may have beneficial effects toward connective tissue comprising elastin outside the heart, such as the pericardium.

PGG may be delivered to a tumor (e.g., a liver tumor or a prostate tumor) or adjacent tissue. Without being limited by theory, the delivery of PGG to the region of the tumor may stabilize by cross-linking, at least transiently, the elastin proteins within the extracellular matrix of the connective tissue. Treatment of the tissue with an elastin-stabilizing compound, such as PGG, may increase the mechanical integrity of the tissue. Treatment with PGG may prevent, inhibit, and/or slow the progression of tumor growth by strengthening the associated tissue. In some instances, treatment with PGG may facilitate natural healing of damaged tissue by mechanically stabilizing the tissue. In some implementations, treatment with PGG may be used prior to, after, and/or concurrently with other interventional treatment, such as surgical intervention. PGG may be delivered to a tumor by devices as described herein, e.g., by an intravascular catheter through a vascular access hole, e.g., in the femoral artery. In some embodiments, PGG, particularly a high purity PGG as disclosed herein, may be suitable for direct injection into the bloodstream or into tumor tissue for treatment. PGG can also be applied topically to the tumor or, after tumor resection, to the tumor bed, to facilitate healing. PGG may have beneficial effects toward connective tissue comprising elastin in regions of a tumor in situ or in regions from which a tumor has been removed.

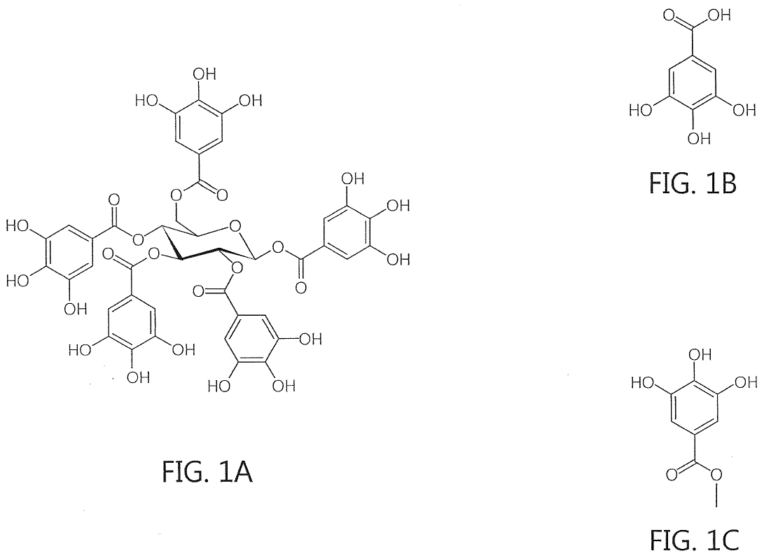

In preferred embodiments, the PGG may be 1,2,3,4,6-pentagalloyl glucose as depicted in FIG. 1A. However, PGG may refer to any chemical structure encompassed by Formula (I):

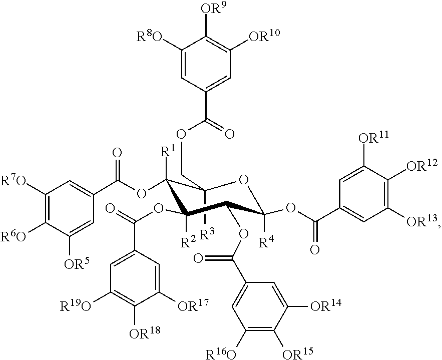

or a pharmaceutically acceptable salt thereof, wherein. R¹-R¹⁹ have any of the values described herein, and wherein the composition is substantially free of gallic acid or methyl gallate. In some embodiments, substantially free is less than about 0.5% gallic acid. In some embodiments, substantially free is less than about 0.5% methyl gallate. In some embodiments, R¹, R², R³ and R⁴ are each independently hydrogen or R^{A}, R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each independently hydrogen or R^{B}; each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R^{Y})₂, —OC(═X)OR^{X}, —NR^{Y}C(═X)R^{Z}, —NR^{Y}C(═X)N(R^{Y})₂, —NR^{Y}C(═X)OR^{X}, unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl; each R^{B} is independently selected from the group consisting of —C(═X)R^{Z}, —C(═X)N(R^{Y})₂, —C(═X)OR^{X}, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, or two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring; each X is independently oxygen (O) or sulfur (S); each R^{X} and R^{Y} is independently selected from the group consisting of hydrogen, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl; and each R^{Z} is independently selected from the group consisting of unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl.

Devices for delivery of PGG or another therapeutic agent to the soft tissue or tendon or vessel or mitral valve to be treated or surgical or implantation site are provided below. Additionally, the devices disclosed herein may be used to delivery any suitable therapeutic agent to the soft tissue or tendon or vessel or surgical or implantation site of a subject. PGG may be delivered to a subject undergoing surgery whereby tendon laxity is prevented, ameliorated, or treated. The devices disclosed herein may also be used to deliver any suitable therapeutic agent to organs or tissue associated with SUI, or implantation site of a subject. PGG may be delivered to a subject directly to treat SUI, even in the absence of surgery or implantation of a device. The devices disclosed herein may be used to delivery any suitable therapeutic agent to the mitral valve, surgical site, or implantation site of a subject. PGG may be delivered to a subject to treat mitral valve disease.

In a preferred embodiment, PGG may be delivered to the soft tissue or tendon or mitral valve or surgical or implantation site to stabilize by cross-linking, at least transiently, the elastin proteins within the extracellular matrix of the connective tissue of the soft tissue or tendon or mitral valve or surgical site. Treatment of the soft tissue or tendon or surgical site with an elastin-stabilizing compound, such as PGG, may increase the mechanical integrity of the soft tissue or tendon or surrounding tissue. Treatment with PGG may prevent, inhibit, slow, and/or reverse the progression of soft tissue disease or tendon laxity or may prevent, inhibit, and/or slow the growth of a peripheral vascular disease. In some instances, treatment with PGG may facilitate natural healing by mechanically stabilizing the soft tissue or tendon. In some implementations, treatment with PGG may be used prior to, after, and/or concurrently with other interventional treatment of the soft tissue or tendon or vessel, such as surgical intervention. In some implementations, treatment with PGG may be used prior to, after, and/or concurrently with surgery, e.g., to prevent or inhibit the development of post-surgical tendon laxity, or prior to, after, and/or concurrently with other interventional treatment of a peripheral vascular disease, such as surgical intervention or angioplasty.

PGG may be delivered to the pelvic organs, urinary tract organs, surgical site, implantation site, or supporting tissue to stabilize by cross-linking, at least transiently, the elastin proteins within the extracellular matrix of the connective tissue of the pelvic or urinary tract organs or supporting tissue, e.g., ligaments and tendons. Treatment of the pelvic or urinary tract organs, surgical site, implantation site, or supporting tissue with an elastin-stabilizing compound, such as PGG, may increase the mechanical integrity of the ligaments and tendons supporting the pelvic or urinary tract organs. Treatment with PGG may prevent, inhibit, and/or slow the progression of SUI or pelvic organ prolapse. In some instances, treatment with PGG may facilitate natural healing by mechanically stabilizing the pelvic or urinary tract organs, surgical site, implantation site, or supporting tissue. In some implementations, treatment with PGG may be used prior to, after, and/or concurrently with other interventional treatment of SUI or pelvic organ prolapse, such as surgical intervention, or may be used to coat or impregnate implantable devices for such treatment.

Treatment with PGG may prevent, inhibit, and/or slow the growth of a mitral valve stenosis. In some instances, treatment with PGG may facilitate natural healing by mechanically stabilizing the mitral valve. In some implementations, treatment with PGG may be used prior to, after, and/or concurrently with other interventional treatment of a mitral valve, such as surgical intervention, e.g., mitral valve repair, replacement, or implantation.

In other applications, PGG may be used to treat soft tissue or a tendon, e.g., a tendon affected by laxity, or a peripheral vascular disease, or a mitral valve disease using another device or route of administration. For instance, in some embodiments, PGG, particularly a high purity PGG as disclosed herein, may be suitable for direct injection into the soft tissue or tendon or vessel, or direct application to the soft tissue or tendon or vessel or mitral valve. PGG may have beneficial effects toward wound closure in connective tissue comprising elastin outside the soft tissue or tendon or vessel or mitral valve, such as the superficial layers of skin above the site of the soft tissue or tendon or vessel, including subcutaneous tissue. PGG may be used to coat grafts, e.g., autologous grafts, used in surgical interventions involving soft tissue or tendons or vessels or mitral valve. PGG may be applied to a surgical site or to the exposed tissue in a surgical procedure, so as to provide a benefit as to inhibition of post-surgical tendon laxity. PGG may be used to coat stents used in the treatment of a peripheral vascular disease or grafts or valves, e.g., as in mitral valve repair, replacement or implantation. A replacement valve, e.g., a biological valve as employed in TAVR, may be coated or impregnated with PGG.

PGG may be used to treat SUI using another device or route of administration than is conventionally employed in treatment of SUI. For instance, in some embodiments, PGG, particularly a high purity PGG as disclosed herein, may be suitable for direct injection into the bloodstream for systemic delivery, into another tissue, e.g., the urinary tract organs or supporting ligaments or tendons, or into the region of a tissue to be treated. In some embodiments, PGG may be used to stabilize and/or facilitate healing of tissues to which a sling or mesh or other supporting structure is attached. In the case of invasive surgery, PGG may promote closure of the surgical access site. The PGG may stabilize tissues around the access hole or incision by crosslinking elastin within the tissues, which may promote or accelerate natural healing. PGG may be applied to the access hole or incision via application to the impacted tissues. PGG may have beneficial effects toward wound closure in other connective tissue comprising elastin, such as the superficial layers of skin impacted by the access hole or incision, including subcutaneous tissue. PGG may be used to coat or impregnate urogynecologic sling or mesh or other implantable structures used in the treatment of SUI, so as to deliver the PGG to surrounding tissue, e.g., in a timed-release manner.

PGG may be used to treat mitral valve disease using another device or route of administration. For instance, in some embodiments, PGG, particularly a high purity PGG as disclosed herein, may be suitable for direct injection into the bloodstream or into another tissue for treatment of mitral valve disease. In some embodiments, PGG may be used to stabilize and/or facilitate closure of vascular access holes associated with minimally invasive surgery for treatment of a mitral valve condition, wherein the holes are created by puncturing a blood vessel for therapeutic treatment via the vasculature, such as delivery of a catheter. PGG may promote closure of the vascular access site. The PGG may stabilize the blood vessel wall around the access hole by crosslinking elastin within the blood vessel, which may promote or accelerate natural healing. PGG may be applied to the access hole via intravascular application and/or by applying PGG directly to the skin over the vascular access hole. PGG may have beneficial effects toward wound closure in connective tissue comprising elastin outside the blood vessel wall, such as the superficial layers of skin above the vascular access hole, including subcutaneous tissue. PGG may be used to coat or impregnate replacement or implantable mitral valves, e.g., biological replacement valves or mechanical valves. If a surgical procedure is performed to repair or replace mitral valves, e.g., open-heart surgery or minimally invasive heart surgery, PGG can be applied to the surgically-repaired tissue, the site of the removed native mitral valve, or tissues in the surgical site or adjacent to the surgical site. In the case of open heart surgery, this can advantageously be accomplished by administering the PGG in solution form via a syringe to the surgical site. In the case of minimally invasive surgery through the vasculature, a weeping balloon as described herein can be employed to deliver the PGG to the surgical or implantation site.

The concentrations of PGG which may be safely delivered to a patient may be generally proportional to the purity of the PGG. For example, gallic acid, depicted in FIG. 1B, and methyl gallate, depicted in FIG. 1C, are common cytotoxic impurities which may be removed from a source batch of PGG during the purification process. Eliminating the presence of or reducing the concentration of toxic impurities from the delivered PGG may allow higher concentrations of the PGG to be delivered due to the mitigation of the toxic side effects of impurities commonly found in isolated PGG. For instance, studies have shown that substantially 100% pure PGG may be safely delivered at concentrations up to approximately 0.330% (w/v), 95% pure PGG may be safely delivered at concentrations up to approximately 0.125% (w/v), and 85% pure PGG may be safely delivered at concentrations up to approximately 0.06% (w/v). Delivery of PGG in higher concentrations may enhance the amount of uptake of PGG by the target tissue which may increase the efficacy of the PGG treatment. Delivery of PGG in higher concentrations may increase the rate of uptake of PGG by the tissue allowing the same amount of uptake in shorter delivery times. Reducing or minimizing the delivery time may be advantageous for reducing the overall treatment time. Minimization of the treatment time may improve the safety and convenience of the treatment procedure and improve patient outcomes.

Unpurified or partially purified PGG may be obtained from any suitable source and purified according to the methods described herein for use as a therapeutic agent. PGG may be extracted from naturally occurring plants such as pomegranate or Chinese gall nut. Extraction and/or isolation methods may entail solvolysis (for example, methanolysis) of tannin or derivative polyphenols as is known in the art. A PGG hydrate is commercially available from Sigma Aldrich (St. Louis, Missouri) at purities greater than or equal to 96%, as confirmed by HPLC. PGG obtained from these sources may undergo additional purification according to the methods described herein to arrive at substantially pure PGG at the purity levels described elsewhere herein.

In some embodiments, PGG is purified by washing a starting batch of PGG (e.g., less than 99% pure) with a solvent. In preferred embodiments, the solvent may comprise diethyl ether. In other embodiments, the solvent may comprise methanol, toluene, isopropyl ether, dichloromethane, methyl tert-butyl ether, 2-butanone, and/or ethyl acetate. In some embodiments, the washing solution may comprise mixtures of the solvents described herein and/or may be mixed with additional solvents. In some embodiments, the starting batch of PGG may be dissolved into a solution. In some embodiments, the PGG may be dissolved in dimethyl sulfoxide (DMSO). In some embodiments, the PGG may be dissolved in any solvent in which the PGG is soluble and which is not miscible with the washing solution. The PGG solution may be mixed with the washing solution in a flask and the PGG solution and washing solution may be allowed to separate over time. The washing solution may subsequently be separated from the PGG solution, such as by draining the denser solution from the flask or by decanting the less dense solution. In some embodiments, the mixture of the washing solution and PGG solution may comprise a volume-to-volume ratio of at least about 1:1, 1.5:1, 2:1, 3:1, 4:1, 5:1, or 10:1 washing solution-to-PGG solution. In some embodiments, the washing step may be repeated at least 1, 2, 3, 4, 5, 6, 7, 8, 9, or 10 times. In some embodiments, the washed PGG solution may be evaporated upon purification to precipitate the PGG into a dry (solid) form. In some embodiments, the PGG may remain dissolved, but the volume of the solution may be increased or decreased (for example, by evaporation). In some embodiments, the starting batch of PGG may be in a dry (solid) form. The PGG may be crystalized. In some embodiments, the PGG may be lyophilized. In some embodiments, the PGG may be precipitated from solution. In some embodiments, the starting batch of PGG may be placed on filter paper and the washing solution poured over the filter paper into a waste flask. The filtration may be facilitated by application of a vacuum to the waste flask (vacuum filtration). Residual washing solution may be evaporated from the purified batch of PGG. In some embodiments, the washing step may be repeated at least 1, 2, 3, 4, 5, 6, 7, 8, 9, or 10 times. The purity of the PGG may increase with each wash. The washing procedure may be repeated until a desired level of purity is attained.

In some embodiments, washing the PGG may result in a purity of at least approximately 99.000%, 99.500%, 99.900%, 99.950%, 99.990%, 99.995%, or 99.999% purity. Purity may be measured as the percent mass (w/w) of PGG in a sample. Purity of the PGG may be measured by any standard means known in the art including chromatography and nuclear magnetic resonance (NMR) spectroscopy. In some embodiments, the purified PGG may comprise no more than approximately 0.001%, 0.005%, 0.01%, 0.05%, 0.1%, 0.2%, 0.3%, 0.4%, 0.5%, 0.6%, 0.7%, 0.8%, 0.9%, or 1% gallic acid. In some embodiments, the purified PGG may comprise no more than approximately 0.001%, 0.005%, 0.01%, 0.05%, 0.1%, 0.2%, 0.3%, 0.4%, 0.5%, 0.6%, 0.7%, 0.8%, 0.9%, or 1% methyl gallate.

PGG may be prepared in a solution for delivery as a therapeutic agent to a patient. The PGG may comprise a purity described elsewhere herein. The PGG may have been purified by the methods disclosed elsewhere herein or may have been purified by other means. In some embodiments, the PGG may be dissolved in a hydrolyzer for subsequent delivery to a patient. The hydrolyzer may comprise any solvent or mixture of solvents in which PGG is readily soluble and which is miscible with water. In some embodiments, the hydrolyzer may be ethanol. In some embodiments, the hydrolyzer may be dimethyl sulfoxide (DMSO). In some embodiments, the hydrolyzer may be contrast media. In some embodiments, the hydrolyzer may be a mixture of ethanol, DMSO, and/or contrast media in any proportions. The hydrolyzer may facilitate the dissolution of PGG into a larger aqueous solution, in which the PGG would not normally be soluble at the same concentration without first being dissolved into the hydrolyzer. The PGG may ultimately be dissolved into a non-toxic aqueous solution suitable for delivery, such as intravascular delivery, to a patient. The aqueous solution may be a saline solution, as is known in the art, or another aqueous solution comprising salts configured to maintain physiological equilibrium with the intravascular environment. The volumetric ratio of the hydrolyzer to the saline solution may be minimized, while maintaining a sufficient volume of hydrolyzer to fully dissolve the desired amount of PGG, to minimize any harmful or toxic effects of the hydrolyzer on the patient, particularly when delivered intravascularly. In some embodiments, the volume-to-volume ratio of saline to hydrolyzer may be no less than about 10:1, 25:1, 50:1, 75:1, 100:1, 200:1, 300:1, 400:1, 500:1, 600:1, 700:1, 800:1, 900:1, or 1000:1. The total volume of the hydrolyzer and saline mixture (including any other additional components) may be configured to prepare the PGG to a desired therapeutic concentration, such as the concentrations described elsewhere herein. In some embodiments, the PGG may be dissolved into the saline or other aqueous solution without a hydrolyzer. In some embodiments, the saline may be warmed (e.g., to above room temperature or above physiological temperature) to dissolve or help dissolve the PGG (or other therapeutic agent). For instance, the saline may be warmed to at least about 25° C., 30° C., 35° C., 40° C., 45° C., 50° C., 55° C., or 60° C. prior to dissolving the PGG. In some implementations, the therapeutic solution may be raised to and/or maintained at an elevated temperature (e.g., physiological temperature) during delivery.

In some embodiments, PGG (for example, purified PGG) for a therapeutic treatment, including but not limited to those described elsewhere herein, may be provided in a kit comprising the components necessary to prepare the PGG for delivery in a therapeutic solution. In some embodiments, the kit may comprise the PGG in a solid (dry) form, the hydrolyzer, and/or the saline solution. The kit may be configured to optimize the storage conditions of the PGG, for short or long-term storage. In some embodiments, the kit may be configured to store the PGG for up to at least 1 week, 2 weeks, 3 weeks, 4 weeks, 1 month, 2 months, 3 months, 4 months, 5 months, 6 months, 1 year, 2 years, or 3 years. The kit may comprise one or more aliquots of each component in pre-measured amounts or volumes. Each component may be provided in a sealed vial, tube, or other container as is known in the art. The containers may each comprise plastic and/or glass. The containers may be configured (for example, tinted or covered) to protect the components from light and/or other radiation. In some embodiments, the kit may be configured for shipping. For example, the components may be contained in a box or other container including desiccants and/or may be configured for temperature control. In some embodiments, the PGG and/or other components may be supplied in a container that has been purged of air (particularly, oxygen). The component may be stored under vacuum or may be purged with an inert gas, such as nitrogen or argon. In some embodiments, the PGG may be mixed with an antioxidant or other stabilizer, in addition to or alternatively to purging the air. In some embodiments, the antioxidant may comprise Vitamin C, Vitamin E, and/or any other antioxidant or stabilizer which is known in the art and is safe for treatment. In some embodiments, the PGG may be provided already dissolved in the hydrolyzer to a predetermined concentration. In some embodiments, the volume of saline provided may be configured to prepare the PGG at a desired therapeutic concentration. In some embodiments, the volume of saline may be configured to prepare the PGG at a maximal therapeutic concentration, such that a user may dilute the PGG with additional solvent to the desired therapeutic concentration. In some embodiments, the total volume of saline may be configured to prepare the PGG at a concentration below the desired concentration and the user may use only a portion of the volume of the saline to prepare the PGG to the desired concentration. The container of saline may have volume indicators for facilitating measurement of the saline. In some embodiments, the saline may be provided in a plurality of aliquots having the same and/or different volumes, which may allow the user to select an aliquot of a desired volume to prepare the PGG at a desired concentration and/or combine various volumes to prepare the PGG at a desired concentration. In some embodiments, the kit may comprise one or more additional components. For example, the kit may comprise a contrast agent for mixing with the therapeutic PGG solution for allowing indirect visualization of the therapeutic solution, as described elsewhere herein.

### LeGoo®

LeGoo® is a tradename of an internal vessel occluder composition produced by Pluromed, Inc. The composition was given FDA approval in 2011 for temporary endovascular occlusion of blood vessels below the neck up to 4 mm in diameter. The composition was not to be used in patients with vascular anatomy or blood flow that precludes cannula placement or proper injection and control of LeGoo.

LeGoo® is comprised of a 20% (weight percent in saline) of purified poloxamer 407, a non-toxic gel which is part of a family of biocompatible, water-soluble polymers that possess reverse, thermosensitive properties (i.e. as temperature increases, viscosity increases). Poloxamer 407 dissolves in blood and is excreted in urine. At room temperature it is a viscous but injectable liquid, and it transitions to a temporary self-forming polymeric plug at body temperature. Because the material undergoes a temperature-induced phase change with no alteration in the product's chemical composition, the material does not “cure” in situ.

LeGoo® is injected into a blood vessel that is intended to be occluded. The amount of LeGoo® injected into the vessel is determined in relationship to the vessel diameter. An arteriotomy is made at a desired location, the cannula is inserted proximally, and LeGoo® is injected against blood flow. When LeGoo® is injected into the blood vessel, the viscosity increases due to the increase in temperature and a plug is formed that occupies space in the vessel, temporarily preventing blood flow. LeGoo® may also be injected distally to stop back bleeding. If left in place and not removed, the plug will dissolve in approximately 15 minutes, or blood flow may be restored by cooling the area with sterile ice or injecting cold saline.

There are two broad categories of vascular occlusion devices available to surgeons to control bleeding: 1. Extravascular occlusion devices; and 2. Intravascular occlusion devices. The mode of action of extravascular occlusive devices is external pressure around the blood vessel. These devices include traditional surgical clamps, clips, vascular (vessel) loops and tapes. The mode of action of intravascular occlusive devices is temporary occlusion of blood flow within a target vessel. Each alternative has its own advantages and disadvantages.

Potential complications may include, but may not be limited to: Effects of transient occlusion of a blood vessel (e.g. infarction, undesired ischemia); Risks associated with the general procedure of clamping a blood vessel (e.g. fibrillation); Risks associated with cannulation (e.g. intimal wall injury.); and Risks associated with application of LeGoo® to epicardial or pericardial surfaces (e.g. adhesions).

LeGoo® is comprised of Poloxamer 407, (also known as Pluronic F127). The conformation of the polymer changes at a certain temperature, the “lower critical solubility temperature” (LCST), or also the “transition temperature.” This conformational change to the somewhat linear polymer allows it to form micelles, which cause an increase in viscosity. If the material is cooled below the transition temperature, then the conformation of the polymer changes back to a somewhat non-linear arrangement and the micelle falls apart. Also, micelles cannot form below a concentration of 12.5%. Once LeGoo® is diluted in blood, the gel plug can no longer occlude the vessel.

Further information about LeGoo® may be in one or more the following U.S. Pat. Nos. 5,800,711, 6,761,824, 8,043,604, 8,361,455, 8,491,623, 8,821,849, 8,998,928, 9,161,767, each of which is hereby incorporated by reference in its entirety.

When LeGoo® is referred to herein, it is understood that other poloxamer gels having similar properties of biocompatibility and reverse thermosensitive properties can also be employed. LeGoo® is suitable for use as a delivery device for PGG or other therapeutic agents, and can advantageously be used to coat or impregnate devices in contact or in proximity to soft tissue or tendons or vessels or surgical sites, and can also be applied directly to soft tissue or tendons or vessels or surgical sites, e.g., for the prevention, amelioration, or treatment of post-surgical tendon laxity or the treatment of vasculature.

The properties of LeGoo® make it adaptable for use in the treatment of SUI or pelvic organ prolapse. For example, LeGoo® can be employed to form a pessary by injection into the vagina, or to form a urethral plug. Alternatively, LeGoo® can be injected into the pelvic cavity around one or more of the urinary tract organs (e.g., kidneys, ureters, bladder, urethra, and sphincter), the vagina, the rectum, or other structures in the pelvic cavity so as to provide support for the urinary tract organs. LeGoo® is also suitable for use as a bulking agent around the urethra. In certain embodiments, support can be provided in the context of reconstructive surgery, or LeGoo® can be employed as a delivery device for PGG. In such an embodiment, the PGG is mixed with or otherwise combined with LeGoo®, such that the PGG elutes into adjacent tissue in vivo. Devices for providing support, e.g., slings or mesh, can be coated with LeGoo®, optionally containing PGG.

### Methods of Treatment

Some embodiments of the present invention include methods of treating a tendon during surgery to prevent, ameliorate, or treat tendon laxity with compositions comprising PGG or other therapeutic agents. Some methods include administering a compound, composition, formulation, or pharmaceutical composition described herein to a subject in need thereof to prevent, ameliorate, or treat tendon laxity. In some embodiments, a subject can be an animal, for example, a mammal such as a human. In some embodiments, the subject is a human.

Some embodiments include methods of treating soft tissue during surgery including tendon repair with compositions comprising PGG or other therapeutic agents. Some embodiments include methods of valve replacement (e.g., mitral valve replacement or TAVR) or implantation (e.g., TAVI) with compositions comprising PGG or other therapeutic agents. Some embodiments include methods of treating SUI or pelvic organ prolapse with compositions comprising PGG or other therapeutic agents. Some methods include administering a compound, composition, formulation, or pharmaceutical composition described herein to a subject in need thereof. In some embodiments, a subject can be an animal, for example, a mammal, a human. In some embodiments, the subject is a human.

Further embodiments include administering a combination of compounds to a subject in need thereof. A combination can include a compound, composition, formulation, or pharmaceutical composition described herein with an additional medicament.

Some embodiments include co-administering a compound, composition, formulation and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered intravenously.

Examples of additional medicaments include collagen crosslinking agents, such as glutaraldehyde, genipin acyl azide, and/or epoxyamine.

In some implementations, PGG and/or other therapeutic agents or medicaments, including but not limited to those described elsewhere herein, may be delivered to the pelvic or urinary tract organs or soft tissue or tendon or ligaments or vasculature or cardiac tissue or surrounding tissue in solution form via syringe or catheter as described herein. In some implementations, PGG and/or other therapeutic agents or medicaments, including but not limited to those described elsewhere herein, may be delivered to tumor tissue or an associated treatment site (e.g., a tumor bed) in solution form via a catheter device as described herein. The delivery catheter may be specifically configured (for example, dimensioned), for delivery of a therapeutic agent to tendons or soft tissue or vasculature or cardiac tissue or a target site (e.g., site of implantation or valve replacement). Commercially available catheters including a lumen can be so employed, with the tip of the catheter positioned in a treatment area and PGG injected through the lumen and out of the tip of the catheter. In other embodiments, the PGG can be administered by topical application to the tendon or surgical site. In the case of a solution of PGG, administration can be dropwise, by spraying, by injection, or by immersion of the soft tissue or tendon or vasculature or cardiac tissue or surgical or implantation site. If the PGG is in a solid form, the soft tissue or tendon or vasculature or cardiac tissue or surgical or implantation site can be dusted with the PGG-containing solid, or the solid can otherwise be placed on or in the soft tissue or tendon or vasculature or cardiac tissue. Selected topical application techniques can advantageously be employed in open surgery where the soft tissue or tendon or vessel or cardiac tissue is exposed. A less invasive procedure may employ injection with a syringe directly into the soft tissue or tendon or vasculature or cardiac tissue or adjacent regions. A less invasive procedure may employ injection with a syringe through the vaginal wall, e.g., directly into the tissue of the ligaments or tendons supporting the urinary tract organs, into the urinary tract organs themselves (e.g., kidneys, ureters, bladder, urethra, or sphincter), or into the pelvic cavity. A less invasive procedure may employ injection with a syringe through the chest wall, e.g., directly into the tissue of the heart, into the pericardial sac, or into the pericardial cavity. In certain embodiments, the PGG is administered in conjunction with other agents, so as to modulate the release of PGG to the tissue (e.g., extended release). LeGoo® or similar poloxamer gels can be employed as a delivery device for PGG. The PGG can be admixed with LeGoo® and then the mixture can be placed on or in a soft tissue or tendon or vessel or valve, on or in a region adjacent to a soft tissue or tendon or vessel or valve, or in a surgical site. PGG can also be delivered systemically, e.g., in an oral or injectable form, as described elsewhere herein.

In certain embodiments, the PGG is delivered to the soft tissue or tendon or vasculature or surgical site or treatment site in an encapsulated form. The encapsulation can degrade, releasing the encapsulated PGG and/or other therapeutic agent over time. For example, the PGG can be entrapped into hydrophilic gelatin microcapsules. Other shell materials include biocompatible water-soluble alcohols and polyethylene oxides. The microencapsulated PGG provides long-term controlled release of PGG at a preselected concentration. LeGoo® or other poloxamer gels are suitable for use as delivery devices for PGG to the soft tissue or tendon or vasculature or surgical site or treatment site. LeGoo® can be employed as an encapsulating agent or delivery vehicle, for PGG and/or other therapeutic agents. In certain embodiments, the PGG is provided in an encapsulated or admixed form with LeGoo®, as described elsewhere herein.

Microencapsulation techniques involve the coating of small solid particles, liquid droplets, or gas bubbles with a thin film of a material, the material providing a protective shell for the contents of the microcapsule. Microcapsules suitable for use in the preferred embodiments may be of any suitable size, typically from about 1 μm or less to about 1000 μm or more, preferably from about 2 μm to about 50, 60, 70, 80, 90, 100, 200, 300, 400, 500, 600, 700, 800, or 900 μm, and more preferably from about 3, 4, 5, 6, 7, 8, or 9 μm to about 10, 15, 20, 25, 30, 35, 40 or 45 μm. In certain embodiments, it may be preferred to use nanometer-sized microcapsules. Such microcapsules may range from about 10 nm or less up to less than about 1000 nm (1 μm), preferably from about 10, 15, 20, 25, 30, 35, 40, 45, 50, 60, 70, 80, or 90 nm up to about 100, 200, 300, 400, 500, 600, 700, 800, or 900 nm.

While in most embodiments a solid phase medicament or other substance is encapsulated, in certain embodiments it may be preferred to incorporate a liquid or gaseous substance. Liquid or gas containing microcapsules may be prepared using conventional methods well known in the art of microcapsule formation.

The microcapsules contain a filling material. The filling material is typically one or more medicaments or other pharmaceutical formulations, e.g., PGG, optionally in combination with substances other than medicaments or pharmaceutical formulations. In certain embodiments, the microcapsules may contain one or more substances not including medicaments or pharmaceutical formulations such as PGG. The filling material is encapsulated within the microcapsule by a shell material.

Typical shell materials include, but are not limited to those pharmaceutically acceptable forms suitable for contact with a tendon, e.g., gum arabic, gelatin, ethylcellulose, polyurea, polyamide, aminoplasts, maltodextrins, and hydrogenated vegetable oil. Gelatin is particularly suitable because of its low cost, biocompatibility, and the ease with which gelatin shell microcapsules may be prepared. In certain embodiments, however, other shell materials may be preferred. The selected shell material may depend upon the particle size and particle size distribution of the filling material, the shape of the filling material particles, compatibility with the filling material, stability of the filling material, and the rate of release of the filling material from the microcapsule.

A variety of encapsulation methods may be used to prepare the microcapsules. These methods include gas phase or vacuum processes wherein a coating is sprayed or otherwise deposited on the filler material particles so as to form a shell, or wherein a liquid is sprayed into a gas phase and is subsequently solidified to produce microcapsules. Suitable methods also include emulsion and dispersion methods wherein the microcapsules are formed in the liquid phase in a reactor.

Encapsulation by spray drying involves spraying a concentrated solution of shell material containing filler material particles or a dispersion of immiscible liquid filler material into a heated chamber where rapid desolvation occurs. Any suitable solvent system may be used, however, the method is most preferred for use with aqueous, ethanolic, or DMSO systems. Spray drying is commonly used to prepare microcapsules including shell materials including, for example, gelatin, hydrolyzed gelatin, gum arabic, modified starch, maltodextrins, sucrose, or sorbitol. When an aqueous solution of shell material is used, the filler material typically includes a hydrophobic liquid or water-immiscible oil. Dispersants and/or emulsifiers may be added to the concentrated solution of shell material. Relatively small microcapsules may be prepared by spray drying methods, e.g., from less than about 1 μm to greater than about 50 μm. The resulting particles may include individual particles as well as aggregates of individual particles. The amount of filler material that may be encapsulated using spray drying techniques is typically from less than about 20 wt. % of the microcapsule to more than 60 wt. % of the microcapsule. The process is preferred because of its low cost compared to other methods, and has wide utility in preparing biocompatible microcapsules. In another variety of spray drying, chilled air rather than desolvation is used to solidify a molten mixture of shell material containing filler material in the form of particles or an immiscible liquid. Various fats, waxes, fatty alcohols, and fatty acids are typically used as shell materials in such an encapsulation method. The method is generally preferred for preparing microcapsules having water-insoluble shells.

Encapsulation using fluidized bed technology involves spraying a liquid shell material, generally in solution or melted form, onto solid particles suspended in a stream of gas, typically heated air, and the particles thus encapsulated are subsequently cooled. Shell materials commonly used include, but are not limited to, colloids, solvent-soluble polymers, and sugars. The shell material may be applied to the particles from the top of the reactor, or may be applied as a spray from the bottom of the reactor, e.g., as in the Wurster process. The particles are maintained in the reactor until a desired shell thickness is achieved. Fluidized bed microencapsulation is commonly used for preparing encapsulated water-soluble food ingredients and pharmaceutical compositions. The method is particularly suitable for coating irregularly shaped particles. Fluidized bed encapsulation is typically used to prepare microcapsules larger than about 100 μm, however smaller microcapsules may also be prepared.

A pair of oppositely charged polyelectrolytes capable of forming a liquid complex coacervate (namely, a mass of colloidal particles that are bound together by electrostatic attraction) can be used to form microcapsules by complex coacervation. A preferred polyanion is gelatin, which is capable of forming complexes with a variety of polyanions. Typical polyanions include gum arabic, polyphosphate, polyacrylic acid, and alginate. Complex coacervation is used primarily to encapsulate water-immiscible liquids or water-insoluble solids. The method is not suitable for use with water soluble substances, or substances sensitive to acidic conditions. In the complex coacervation of gelatin with gum arabic, a water insoluble filler material is dispersed in a warm aqueous gelatin emulsion, and then gum arabic and water are added to this emulsion. The pH of the aqueous phase is adjusted to slightly acidic, thereby forming the complex coacervate which adsorbs on the surface of the filler material. The system is cooled, and a cross-linking agent, such as glutaraldehyde, is added. The microcapsules may optionally be treated with urea and formaldehyde at low pH so as to reduce the hydrophilicity of the shell, thereby facilitating drying without excessive aggregate formation. The resulting microcapsules may then be dried to form a powder.

Microcapsules may be prepared using a solution containing two liquid polymers that are incompatible, but soluble in a common solvent. One of the polymers is preferentially absorbed by the filler material. When the filler material is dispersed in the solution, it is spontaneously coated by a thin film of the polymer that is preferentially absorbed. The microcapsules are obtained by either crosslinking the absorbed polymer or by adding a nonsolvent for the polymer to the solution. The liquids are then removed to obtain the microcapsules in the form of a dry powder.

Polymer-polymer incompatibility encapsulation can be carried out in aqueous or nonaqueous media. It is typically used for preparing microcapsules containing polar solids with limited water solubility. Suitable shell materials include ethylcellulose, polylactide, and lactide-glycolide copolymers. Polymer-polymer incompatibility encapsulation is often preferred for encapsulating oral and parenteral pharmaceutical compositions, especially those containing proteins or polypeptides, because biodegradable microcapsules may be easily prepared. Microcapsules prepared by polymer-polymer incompatibility encapsulation tend to be smaller than microcapsules prepared by other methods, and typically have diameters of 100 μm or less.

Microcapsules may be prepared by conducting polymerization reactions at interfaces in a liquid. In one such type of microencapsulation method, a dispersion of two immiscible liquids is prepared. The dispersed phase forms the filler material. Each phase contains a separate reactant, the reactants capable of undergoing a polymerization reaction to form a shell. The reactant in the dispersed phase and the reactant in a continuous phase react at the interface between the dispersed phase and the continuous phase to form a shell. The reactant in the continuous phase is typically conducted to the interface by a diffusion process. Once reaction is initiated, the shell eventually becomes a barrier to diffusion and thereby limits the rate of the interfacial polymerization reaction. This may affect the morphology and uniformity of thickness of the shell. Dispersants may be added to the continuous phase. The dispersed phase can include an aqueous or a nonaqueous solvent. The continuous phase is selected to be immiscible in the dispersed phase.

Typical polymerization reactants may include acid chlorides or isocyanates, which are capable of undergoing a polymerization reaction with amines or alcohols. The amine or alcohol is solubilized in the aqueous phase in a nonaqueous phase capable solubilizing the amine or alcohol. The acid chloride or isocyanate is then dissolved in the water- (or nonaqueous solvent-) immiscible phase. Similarly, solid particles containing reactants or having reactants coated on the surface may be dispersed in a liquid in which the solid particles are not substantially soluble. The reactants in or on the solid particles then react with reactants in the continuous phase to form a shell.

In another type of microencapsulation by interfacial polymerization, commonly referred to as in situ encapsulation, a filler material in the form of substantially insoluble particles or in the form of a water immiscible liquid is dispersed in an aqueous phase. The aqueous phase contains urea, melamine, water-soluble urea-formaldehyde condensate, or water-soluble urea-melamine condensate. To form a shell encapsulating the filler material, formaldehyde is added to the aqueous phase, which is heated and acidified. A condensation product then deposits on the surface of the dispersed core material as the polymerization reaction progresses. Unlike the interfacial polymerization reaction described above, the method may be suitable for use with sensitive filler materials since reactive agents do not have to be dissolved in the filler material. In a related in situ polymerization method, a water-immiscible liquid or solid containing a water-immiscible vinyl monomer and vinyl monomer initiator is dispersed in an aqueous phase. Polymerization is initiated by heating and a vinyl shell is produced at the interface with the aqueous phase.

Microcapsules may be prepared by removing a volatile solvent from an emulsion of two immiscible liquids, e.g., an oil-in-water, oil-in-oil, or water-in-oil-in-water emulsion. The material that forms the shell is soluble in the volatile solvent. The filler material is dissolved, dispersed, or emulsified in the solution. Suitable solvents include methylene chloride and ethyl acetate. Solvent evaporation is a preferred method for encapsulating water soluble filler materials, for example, polypeptides. When such water-soluble components are to be encapsulated, a thickening agent is typically added to the aqueous phase, then the solution is cooled to gel the aqueous phase before the solvent is removed. Dispersing agents may also be added to the emulsion prior to solvent removal. Solvent is typically removed by evaporation at atmospheric or reduced pressure. Microcapsules less than 1 μm or over 1000 μm in diameter may be prepared using solvent evaporation methods.

Microencapsulation by centrifugal force typically utilizes a perforated cup containing an emulsion of shell and filler material. The cup is immersed in an oil bath and spun at a fixed rate, whereby droplets including the shell and filler material form in the oil outside the spinning cup. The droplets are gelled by cooling to yield oil-loaded particles that may be subsequently dried. The microcapsules thus produced are generally relatively large. In another variation of centrifugal force encapsulation referred to as rotational suspension separation, a mixture of filler material particles and either molten shell or a solution of shell material is fed onto a rotating disk. Coated particles are flung off the edge of the disk, where they are gelled or desolvated and collected.

Microencapsulation by submerged nozzle generally involves spraying a liquid mixture of shell and filler material through a nozzle into a stream of carrier fluid. The resulting droplets are gelled and cooled. The microcapsules thus produced are generally relatively large.

In desolvation or extractive drying, a dispersion filler material in a concentrated shell material solution or dispersion is atomized into a desolvation solvent, typically a water-miscible alcohol when an aqueous dispersion is used. Water-soluble shell materials are typically used, including maltodextrins, sugars, and gums. Preferred desolvation solvents include water-miscible alcohols such as 2-propanol or polyglycols. The resulting microcapsules do not have a distinct filler material phase. Microcapsules thus produced typically contain less than about 15 wt. % filler material, but in certain embodiments may contain more filler material.

Liposomes are microparticles typically ranging in size from less than about 30 nm to greater than 1 mm. They consist of a bilayer of phospholipid encapsulating an aqueous space. The lipid molecules arrange themselves by exposing their polar head groups toward the aqueous phase, and the hydrophobic hydrocarbon groups adhere together in the bilayer forming close concentric lipid leaflets separating aqueous regions. Medicaments can either be encapsulated in the aqueous space or entrapped between the lipid bilayers. Where the medicament is encapsulated depends upon its physiochemical characteristics and the composition of the lipid. Liposomes may slowly release any contained medicament through enzymatic hydrolysis of the lipid.

While the microencapsulation methods described above are generally applicable for preparing the microcapsules of certain embodiments, other suitable microencapsulation methods may also be used, as are known to those of skill in the art. Moreover, in certain embodiments, it may be desired to administer an unencapsulated medicament, e.g., PGG, or other substance directly to or in the tendon or soft tissue or vasculature. Alternatively, the medicament, e.g., PGG, or other substance may be incorporated into a solid or gel matrix of a carrier substance and implanted in the body so as to elute PGG into a treatment region (e.g., a soft tissue or tendon or vessel or surgical site or treatment site or implantation site).

The PGG can be administered in a purified form, as described elsewhere herein, or can be present in a formulation with one or more additional therapeutic agents or pharmaceutical excipients.

In certain embodiments, the PGG can be administered in an intravenous form, either as an injection for systemic administration or via a delivery catheter or syringe directly to a treatment site; however, other routes of administration are also contemplated. Contemplated methods of administration include but are not limited to orally, subcutaneously, intravenously, intranasally, topically, transdermally, intraperitoneally, intramuscularly, intrapulmonarilly, vaginally, rectally, or intraocularly. The PGG can be formulated into liquid preparations for, e.g., oral administration. Suitable forms include suspensions, syrups, elixirs, and the like. Unit dosage forms for oral administration include tablets and capsules. Unit dosage forms configured for administration once a day can be employed; however, in certain embodiments it can be desirable to configure the unit dosage form for administration twice a day, or more.

The PGG can be formulated into pharmaceutical compositions for use in treatment of a soft tissue or vasculature or tendon or surgical sites or to facilitate recovery of patients undergoing surgery, e.g., tendon repair, e.g., whereby post-surgical tendon laxity is prevented, ameliorated, or treated, or tendon or soft tissue healing is promoted, or the vasculature is treated. The PGG can be formulated into pharmaceutical compositions for use in treatment of congestive heart failure. The PGG can be formulated into pharmaceutical compositions for use in treatment of SUI or pelvic organ prolapse. The PGG can be formulated into pharmaceutical compositions for use in treatment of tumors or to facilitate recovery of patients undergoing conventional treatments for tumors (e.g., surgery, chemotherapy, cryotherapy, chemical therapy, or radiation therapy). Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of PGG or a compound described herein (including enantiomers, diastereoisomers, tautomers, polymorphs, and solvates thereof), or pharmaceutically acceptable salts thereof, and (b) a pharmaceutically acceptable carrier, diluent, excipient or combination thereof. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, for example, in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions. Tonicity adjustors may be added as needed or convenient. They include, but are not limited to, salts, particularly sodium chloride, potassium chloride, mannitol and glycerin, or any other suitable ophthalmically acceptable tonicity adjustor. Various buffers and means for adjusting pH may be used so long as the resulting preparation is pharmaceutically acceptable. For many compositions, the pH will be between 4 and 9. Accordingly, buffers include acetate buffers, citrate buffers, phosphate buffers and borate buffers. Acids or bases may be used to adjust the pH of these formulations as needed. Acceptable antioxidants include, but are not limited to, sodium metabisulfite, sodium thiosulfate, acetylcysteine, butylated hydroxyanisole and butylated hydroxytoluene. A useful chelating agent is edetate disodium, although other chelating agents may also be used in place or in conjunction with it. Topical formulations may generally be comprised of a pharmaceutical carrier, co-solvent, emulsifier, penetration enhancer, preservative system, and emollient.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (for example, from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The PGG compositions in liquid form are preferably isotonic with the blood or other body fluid of the recipient. The isotonicity of the compositions can be attained using sodium tartrate, propylene glycol or other inorganic or organic solutes. Sodium chloride is advantageously employed. Buffering agents can be employed, such as acetic acid and salts, citric acid and salts, boric acid and salts, and phosphoric acid and salts. Parenteral and vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In some embodiments it can be desirable to include a reducing agent, such as vitamin C, vitamin E, or other reducing agents as are known in the pharmaceutical arts, in the formulation.

For intravenous, intra-pelvic cavity, intra-tissue administration, or parenteral administration, the PGG and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous, intra-pelvic cavity, intra-tissue administration, or parenteral administration may be provided in the form of one more solids that are reconstituted with a suitable diluent such as sterile water, saline or dextrose in water shortly prior to administration. In other embodiments, the compositions are provided in solution ready to administer intravascularly, intravenously, topically, or systemically. In still other embodiments, the compositions are provided in a solution that is further diluted prior to administration. In embodiments that include administering a combination of PGG and another agent, the combination may be provided as a mixture, or the caregivers may mix the two agents prior to administration, or the two agents may be administered separately.

The actual dose of the PGG described herein depends on the specific compound, and on the condition to be treated. In some embodiments wherein PGG is administered systemically, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day.

The PGG formulation can be in a solid form, or in a viscous liquid form. Viscosity of the formulation can be maintained at the selected level using a pharmaceutically acceptable thickening agent. Methylcellulose is readily available and is easy to work with. Other suitable thickening agents include, for example, xanthan gum, carboxymethyl cellulose, hydroxypropyl cellulose, carbomer, and the like. The concentration of the thickener will depend upon the thickening agent selected. An amount is typically used that will achieve the selected viscosity. Viscous compositions are normally prepared from solutions by the addition of such thickening agents.

A pharmaceutically acceptable preservative can be employed to increase the shelf life of the PGG compositions. Benzyl alcohol can be suitable, although a variety of preservatives including, for example, parabens, thimerosal, chlorobutanol, or benzalkonium chloride can also be employed. A suitable concentration of the preservative is typically from about 0.02% to about 2% based on the total weight of the composition, although larger or smaller amounts can be desirable depending upon the agent selected. Reducing agents, as described above, can be advantageously used to maintain good shelf life of the formulation.

The PGG can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

For oral administration, the PGG can be provided as a tablet, aqueous or oil suspension, dispersible powder or granule, emulsion, hard or soft capsule, syrup or elixir. Compositions intended for oral use can be prepared according to any method known in the art for the manufacture of pharmaceutical compositions and can include one or more of the following agents: sweeteners, flavoring agents, coloring agents and preservatives. Aqueous suspensions can contain the active ingredient in admixture with excipients suitable for the manufacture of aqueous suspensions.

PGG formulations for oral use can be solid forms as tablets, capsules, granules and bulk powders, or can be provided as hard gelatin capsules, wherein the active ingredient(s) are mixed with an inert solid diluent, such as calcium carbonate, calcium phosphate, or kaolin, or as soft gelatin capsules. In soft capsules, the active compounds can be dissolved or suspended in suitable liquids, such as water or an oil medium, such as peanut oil, olive oil, fatty oils, liquid paraffin, or liquid polyethylene glycols. Stabilizers and microspheres formulated for oral administration can also be used. Capsules can include push-fit capsules made of gelatin, as well as soft, sealed capsules made of gelatin and a plasticizer, such as glycerol or sorbitol. The push-fit capsules can contain the active ingredient in admixture with fillers such as lactose, binders such as starches, and/or lubricants such as talc or magnesium stearate and, optionally, stabilizers.

Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents. Tablets can be uncoated or coated by known methods to delay disintegration and absorption in the gastrointestinal tract and thereby provide a sustained action over a longer period of time. For example, a time delay material such as glyceryl monostearate can be used. When administered in solid form, such as tablet form, the solid form typically comprises from about 0.001 wt. % or less to about 50 wt. % or more of active ingredient(s), preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets can contain the PGG in admixture with non-toxic pharmaceutically acceptable excipients including inert materials. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art. For example, a tablet can be prepared by compression or molding, optionally, with one or more additional ingredients. Compressed tablets can be prepared by compressing in a suitable machine the active ingredients in a free-flowing form such as powder or granules, optionally mixed with a binder, lubricant, inert diluent, surface active or dispersing agent. Molded tablets can be made by molding, in a suitable machine, a mixture of the powdered compound moistened with an inert liquid diluent.

Preferably, each tablet or capsule contains from about 10 mg or less to about 1,000 mg or more of a compound of the PGG, more preferably from about 20, 30, 40, 50, 60, 70, 80, 90, or 100 mg to about 150, 200, 250, 300, 350, 400,450, 500, 550, 600, 650, 700, 750, 800, or 900 mg. Most preferably, tablets or capsules are provided in a range of dosages to permit divided dosages to be administered. A dosage appropriate to the patient and the number of doses to be administered daily can thus be conveniently selected. In certain embodiments it can be preferred to incorporate the PGG and one or more other therapeutic agents to be administered into a single tablet or other dosage form (e.g., in a combination therapy); however, in other embodiments it can be preferred to provide the PGG and other therapeutic agents in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents can be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, karaya or tragacanth, or alginic acid or salts thereof.

Binders can be used to form a hard tablet. Binders include materials from natural products such as acacia, tragacanth, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, can be included in tablet formulations.

Surfactants can also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations of PGG can be employed wherein the PGG is incorporated into an inert matrix that permits release by either diffusion or leaching mechanisms, e.g., a microencapsulated form. Slowly degenerating matrices can also be incorporated into the formulation. Other delivery systems can include timed release, delayed release, or sustained release delivery systems.

Coatings can be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments can be added for identification or to characterize different combinations of active compound doses. Coatings can include pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

When administered orally in liquid form, a liquid carrier such as water, petroleum, oils of animal or plant origin such as peanut oil, mineral oil, soybean oil, or sesame oil, or synthetic oils can be added to the active ingredient(s). Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above. Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain sweetening and flavoring agents. Preservatives that may be used in the pharmaceutical compositions disclosed herein include, but are not limited to, benzalkonium chloride, PHFMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

When PGG is administered by intravenous, parenteral, or other injection, it is preferably in the form of a pyrogen-free, parenterally acceptable aqueous solution, alcoholic solution (e.g., ethanolic solution), or oleaginous suspension. Suspensions can be formulated according to methods well known in the art using suitable dispersing or wetting agents and suspending agents. The preparation of acceptable aqueous solutions with suitable pH, isotonicity, stability, and the like, is within the skill in the art. A preferred pharmaceutical composition for injection preferably contains an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils can be employed conventionally as a solvent or suspending medium. For this purpose, any bland fixed oil can be employed including synthetic mono or diglycerides. In addition, fatty acids such as oleic acid can likewise be used in the formation of injectable preparations. The pharmaceutical compositions can also contain stabilizers, preservatives, buffers, antioxidants, or other additives known to those of skill in the art.

The duration of an intravenous injection or other injection can be adjusted depending upon various factors, and can comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous or other injection administration.

The PGG compositions of the preferred embodiments can additionally employ adjunct components conventionally found in pharmaceutical compositions in their art-established fashion and at their art-established levels. Thus, for example, the compositions can contain additional compatible pharmaceutically active materials for therapy (such as antimicrobials, local anesthetics, anti-inflammatory agents, and the like), or can contain materials useful in physically formulating various dosage forms of the preferred embodiments, such as excipients, dyes, thickening agents, stabilizers, preservatives or antioxidants.

The compounds of the preferred embodiments can be provided to an administering physician or other health care professional in the form of a kit. The kit is a package which houses a container that contains the PGG and optionally other compound(s) in a suitable pharmaceutical composition, and instructions for administering the pharmaceutical composition to a subject. The kit can optionally also contain one or more additional therapeutic agents, e.g., antimicrobials or anesthetics, or therapeutic agents. For example, a kit containing PGG in combination with one or more additional agents can be provided, or separate pharmaceutical compositions containing therapeutic agents can be provided. The kit can also contain separate doses of PGG for serial or sequential administration. The PGG can optionally be admixed with LeGoo® or a similar poloxamer gel to facilitate delivery of PGG to a tendon or surgical site. The kit can optionally contain one or more diagnostic tools and instructions for use. The kit can contain suitable delivery devices, e.g., syringes, a delivery catheter, or the like, along with instructions for administering the PGG and any other therapeutic agent. The kit can optionally contain instructions for storage, reconstitution (if applicable), and administration of any or all therapeutic agents included. The kits can include a plurality of containers reflecting the number of administrations to be given to a subject.

In a kit for treating deep vein thrombosis, the other therapeutic agents can include, e.g., blood thinners such as heparin, enoxaparin (Lovenox), dalteparin (Fragmin), fondaparinux (Arixtra), warfarin (Coumadin, Jantoven), dabigatran (Pradaxa), rivaroxaban (Xarelto), apixaban (Eliquis), or edoxaban (Savaysa). In a kit for treating PVD, the other therapeutic agents can include, e.g., cilostazol or pentoxifylline to increase blood flow and relieve symptoms of claudication, clopidogrel or daily aspirin to reduce blood clotting, atorvastatin, simvastatin, or other statins to lower high cholesterol, angiotensin-converting enzyme (ACE) inhibitors to lower high blood pressure, or diabetes medication to control blood sugar in diabetics. For chronic venous insufficiency, the additional therapeutic agents can include, e.g., antibiotics to clear skin infections or treat deeper infections or ulcers related to CVI, medication to prevent the formation of additional blood clots, or the herbal dietary supplement Vena-Stat. In one embodiment, a kit for the treatment of congestive heart failure is provided that includes PGG and one or more therapeutic agents currently employed for the treatment congestive heart failure. In one embodiment, a kit for the treatment of SUI is provided that includes PGG and one or more therapeutic agents. Therapeutic agents for treating SUI include oxybutynin (Ditropan XL), tolterodine (Detrol), darifenacin (Enablex), fesoterodine (Toviaz), solifenacin (Vesicare), trospium (Sanctura), and Mirabegron (Myrbetriq). In the treatment of congestive heart failure, the kit can optionally also contain one or more additional therapeutic agents, e.g., antimicrobials or anesthetics, or therapeutic agents for treating congestive heart failure, e.g., beta blockers (carvedilol, metoprolol), ACE inhibitors (lisinopril, captopril), angiotensin receptor blockers (losartan), aldosterone antagonists (spirolactone, eplerenone), digoxin (lanoxin), hydralazine and nitrates (apresoline, nitrobid, imdur, isordil), and diuretics (furosemide, bumetanide, torsemide, metolazone). In embodiments for the treatment of tumors, a kit for the treatment of tumors is provided that includes PGG and one or more therapeutic agents currently employed for tumors. The kit can optionally also contain one or more additional therapeutic agents, e.g., antimicrobials or anesthetics, or chemotherapeutic agents, e.g., daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES).

A kit containing PGG in combination with one or more additional agents can be provided, or separate pharmaceutical compositions containing a compound of the preferred embodiments and additional therapeutic agents can be provided. The kit can also contain separate doses of PGG for serial or sequential administration. The kit can optionally contain one or more diagnostic tools and instructions for use. The kit can contain suitable delivery devices, e.g., syringes, a delivery catheter, or the like, along with instructions for administering the PGG and any other therapeutic agent. The kit can optionally contain instructions for storage, reconstitution (if applicable), and administration of any or all therapeutic agents included. The kits can include a plurality of containers reflecting the number of administrations to be given to a subject. As described above, in some implementations, a catheter or syringe delivers PGG, optionally with other therapeutic agents or pharmaceutically acceptable components, to the tendon, soft tissue, vasculature, cardiac tissue, surgical site, or to the urinary tract organs, the pelvic cavity, supporting tendons and ligaments, or other target site. In other embodiments, PGG in solid form is dusted or otherwise placed on or in tendon, soft tissue, a vessel or body lumen, cardiac tissue, a target site, or a surgical site. Variations of the procedure described herein may be encompassed. In some implementations, a device different from a syringe or delivery catheter may be used. In some implementations, a therapeutic agent other than or in addition to PGG may be delivered. In some implementations, the therapeutic agent may be delivered systemically rather than administered directly to the tendon or soft tissue or vasculature or cardiac tissue or a tumor. In some implementations, the treatment may be applied prophylactically to otherwise healthy tissue to prevent tendon laxity in a patient having risk factors or may be applied prophylactically to otherwise healthy vasculature or adjacent tissue to prevent congestive heart failure, peripheral vascular disease (e.g., peripheral arterial disease), chronic venous insufficiency, deep vein thrombosis, and varicose veins in a patient having a different disease condition that is a risk factor for the condition. The treatment can also be applied in a structure adjacent to the soft tissue or tendon or vasculature, e.g., a surgical site, to target the cellular or extracellular environment adjacent the soft tissue or tendon or vasculature or cardiac tissue, e.g., so as to prevent, ameliorate, or treat tendon laxity or to facilitate healing of tendons or soft tissue or vasculature or cardiac tissue. In some implementations, the treatment may be applied prophylactically to otherwise healthy tissue to prevent SUI in a patient having a risk factor for SUI. In some implementations, the treatment may be applied to tissue to slow the progression of SUI in a patient at early stages of SUI. The treatment can also be applied in a structure adjacent to urinary tract organ tissue (e.g., in the pelvic cavity, or to ligaments or tendons supporting the urinary tract organs) to target the cellular or extracellular environment in the pelvis. In some implementations, the treatment may be applied prophylactically to otherwise healthy tissue to prevent tumor formation in a patient having a different disease condition that is a risk factor for tumor formation. The treatment can also be applied in a structure adjacent to tumor tissue (e.g., associated vasculature, cysts or liquid areas, normal tissue surrounding the tumor, a tumor bed, or a region of a tumor) to target the cellular or extracellular environment adjacent the tumor.

Described herein are conventional methods for tendon treatment or repair or treatment of other soft tissue, e.g., to prevent, ameliorate, or treat tendon laxity, such as post-surgical tendon laxity. In the case of surgical intervention, PGG in suitable form can be applied to tissues adjacent to the surgical site or to the tendon itself, or in situ soft tissue (e.g., tendon), soft tissue to be transplanted (e.g., tendon), soft tissue grafts (e.g., autologous grafts), tendon grafts, or the like. The PGG can be administered, before, during, or after surgery or a procedure.

Described herein are conventional methods for treatment of tumors. In the case of surgical intervention or ablative therapies, PGG in suitable form can be applied to tissues adjacent to the removed, resected, or destroyed tumor. In chemical ablation therapies, PGG can be added to the chemical employed. If chemotherapy (e.g., via intraarterial catheter) or radiation beads are delivered to the patient, PGG in suitable form can be added to the administered composition.

In some embodiments, the therapeutic agent may be PGG. The PGG may be dissolved in solution at a final concentration that is no less than approximately 0.01%, 0.05%, 0.1%, 0.2%, 0.3%, 0.4%, 0.5%, 0.6%, 0.7%, 0.8%, 0.9%, or 1% (w/v), for either topical administration or by injection for systemic administration, e.g., in LeGoo® or another poloxamer gel. As described elsewhere herein, higher concentrations of PGG may provide for more effective treatment, especially over shorter treatment times, and are particularly suited for localized administration (e.g., topical administration). Accordingly, higher concentrations may allow shorter treatment time. Higher purity PGG may be less toxic, due to absence of toxic impurities, than lower purity PGG. Accordingly, higher purity PGG may be safer to user at higher concentrations than lower purity PGG. The PGG may be dissolved in an inflation fluid such as saline (for example, via a hydrolyzer as described elsewhere herein). The volume of delivered therapeutic fluid may be no more than approximately 150 mL, 125 mL, 100 mL, 75 mL, 50 mL, 40 mL, 30 mL, 20 mL, 15 mL, 10 mL, 8 mL, 5 mL, 3 mL, or 1 mL. In some embodiments, more than 1 mL may be administered. The duration of delivery may be no more than about 30 min, 10 min, 5 min, 4 min, 3 min, 2 min, 1 min, 45 seconds, 30 seconds, 20 seconds, or 10 seconds. In some embodiments, e.g., extended release, the duration of delivery may be more than about 30 minutes. The precise volume of delivered fluid and/or the duration of delivery may depend on the size of the surgical area or target area or soft tissue or tendon or vasculature or urinary tract organ to be treated.

Surgical methods of treatment for PVD or PAD include angioplasty or vascular surgery (e.g., including implantation of stents or grafts). The balloon-equipped catheters described herein are suitable for use in both inflating to open the artery and to deliver PGG or other therapeutic agents to the vasculature. In certain embodiments, the catheters, stents, or grafts can be coated or impregnated with LeGoo® (optionally as a delivery device for PGG), or LeGoo® (optionally containing PGG) can be employed to temporarily occlude a blood vessel in conjunction with the angioplasty or vascular surgery.

For chronic venous insufficiency or varicose veins, sclerotherapy or endovenous thermal ablation can be performed, with PGG being applied to the remaining venous tissue and surrounding areas to facilitate treatment. PGG can also be applied to venous tissue or surrounding tissue in vein ligation and stripping, microincision/ambulatory phlebectomy, and bypass surgery.

### Angioplasty

In some embodiments, angioplasty can be conducted as a treatment for peripheral vascular disease. Alternatively, a stent may be placed in an affected vessel. Surgery can also be conducted. In some embodiments, a balloon may be configured to deliver a therapeutic agent, such as a PGG solution, to the implantation or surgical site in a mitral valve repair or replacement. In some embodiments, a balloon may be configured to deliver a therapeutic agent, such as a PGG solution, to the surgical site in a TAVR or TAVI. The balloon may be what is known in the art as a weeping balloon. In such treatment methods, a balloon may be configured to deliver a therapeutic agent, such as a PGG solution, to the treatment, implantation or surgical site. The balloon may be what is known in the art as a weeping balloon. The balloon may comprise a plurality of pores disposed in the expandable membrane of the balloon configured to place the interior volume of the balloon in fluid communication with the intravascular environment. The solution of therapeutic agent may be used as the inflation fluid. The pores may be configured to provide fluid communication between the interior volume of the balloon and the intravascular environment while allowing for pressurization and inflation of the balloon. In some embodiments, the size of the pores may increase as the expandable membrane of the balloon expands. The elastic properties of the expandable membrane of the balloon may allow for a continuous expansion of the pore size of the pores as the interior volume of the balloon is increased causing the expandable membrane to stretch. The volumetric flow rate at which the inflation fluid escapes from the interior volume of the balloon into the intravascular environment may increase as the balloon expands. In some embodiments, the pores may allow for a constant or substantially constant volumetric flow rate of fluid across the pores over a range of pressures of the interior volume. The volumetric flow rate out of the balloon may be maximized at a certain level of pressurization or volumetric flow rates of inflation fluid into the balloon. The inflation fluid may be introduced into the interior volume of the balloon at a volumetric flow rate that is greater than the volumetric flow rate at which the inflation fluid flows through the pores, such that the balloon may be inflated even while fluid escapes or leaks through the pores. In some implementations, the balloon may be inflated using an inflation fluid (for example, saline) that does not comprise the therapeutic agent. The inflation fluid may be switched over to the therapeutic solution or the therapeutic agent may be added to the inflation fluid after the treatment, implantation or surgical site has been sealed from retrograde blood flow. Staggering the delivery of the therapeutic agent may conserve the therapeutic agent and/or may prevent, reduce, or minimize the amount of therapeutic agent that is released into the blood stream before the fluid seal is fully formed within the target site to be treated, e.g., a surgical site, e.g., in TAVR or TAVI or of a removed native mitral valve or the site of a mitral valve to be repaired or otherwise treated.

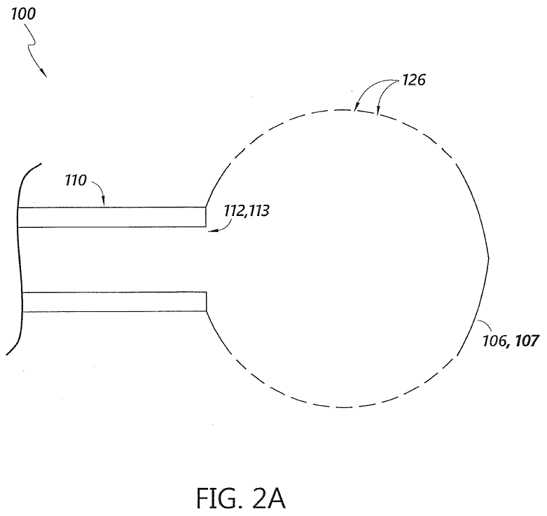

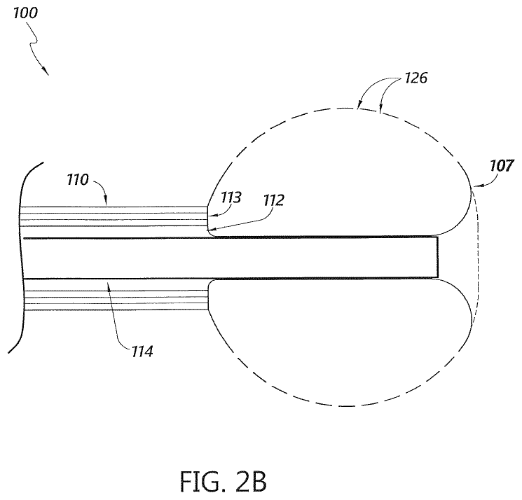

FIG. 2A schematically depicts an example of a weeping balloon. The delivery catheter 100 may comprise a proximal end (not shown), configured to remain outside of the body during use. The delivery catheter 100 may comprise a main shaft 110 and an expandable member 106,107 comprising a plurality of pores 126. Such a configuration is useful for introduction of the delivery catheter 100 from a vascular access point distant from the treatment site. The balloon of FIG. 2A is suitable for use in a balloon angioplasty or balloon valvuloplasty, or can be adapted to support an implantable or replacement mitral valve.

The expandable member 106,107 may comprise an expanded configuration having an expanded radial diameter and an unexpanded configuration having an unexpanded radial diameter, the expanded radial diameter being larger than the unexpanded radial diameter. The length of the expandable member 106,107 may increase, decrease, or remain the same upon expansion. The unexpanded diameter of the expandable member 106,107 may be configured to facilitate insertion of the delivery catheter 100 into the treatment site. The unexpanded diameters may each be less than, approximately the same as, or larger than an inner diameter and/or outer diameter of the main shaft 110. The expanded diameter of the expandable member 106,107 may be configured to occlude the target site and may be the same as or larger than the diameter of target site vessel. In some embodiments, the expandable member 106,107 may be operable at intermediate diameters between the unexpanded and fully expanded diameter.

In various embodiments, the expandable member 106,107 may be an inflatable balloon 107, also shown in FIG. 2A. The inflatable balloon 107 may comprise an elastic material forming an expandable membrane as is known in the art and may be configured to expand upon pressurization from an inflation fluid (for example, a gas or a liquid, such as saline). The balloon material may be biocompatible. In some embodiments, the expandable member 106 may be expandable through means other than or in addition to inflation. For example, the expandable member 106 may comprise a radially expandable frame. The expandable frame may comprise a shape memory material (for example, a nickel titanium alloy (nitinol)) and/or may be configured to self-expand. The expandable member 106,107 may be configured to self-expand upon release of a constraining mechanism, such as an outer sheath surrounding the expandable member, which may, for instance, be proximally withdrawn to allow self-expansion of the expandable member. In some embodiments, the expandable frame may be configured to be mechanically expanded, such as by a push wire or pull wire extending through an internal lumen of the delivery catheter 100. The expandable frames may be fixed or coupled to a surrounding fluid impermeable covering or coating such that the expandable member 106,107 may be configured to occlude fluid flow as described elsewhere herein.

The main shaft 110 may comprise a length and a diameter configured to facilitate navigation of the expandable member 106,107 to the target site. In some embodiments, the diameter may vary over a length of the main shaft 110 and/or any internal components, including internal shafts described elsewhere herein. For example, the diameter may decrease in a proximal to distal direction causing a distal portion of the delivery catheter 100 to be more flexible than a proximal portion. The main shaft 110 may be generally tubular having a sidewall forming the lumen 112. The lumen 112 may serve as an inflation lumen 113 for inflating and/or deflating the expandable member 106,107. An inflation fluid (for example, saline, for example, containing PGG) may be introduced from a proximal end of the delivery catheter 100 through the inflation lumen 113 into the interior volume of the expandable member 106 and removed (for example, aspirated from the expandable member 106,107) through the inflation lumen 113 to de-inflate the expandable member 106,107. The proximal end of the inflation lumen 113 and/or any other inflation lumens described herein may each be in fluid communication with a source of pressurized inflation fluid, such as a syringe, an IV bag, a fluid pump, etc. One or more of the inflation lumens and/or balloons described herein may be in fluid communication with one or more pressure sensors for monitoring pressure levels within the internal lumens and/or the balloons with which they are in fluid communication. In some embodiments in which the expandable member 106 comprises an expandable frame, a pull wire or push wire may extend through the first inflation lumen 113 for actuating the expansion or compression of the expandable member 106,107.

In some embodiments, as depicted in FIG. 2A, the balloon 107 may comprise an expandable membrane having a proximal end and a distal end. The proximal end of the expandable membrane may be coupled to (for example, at or near) the distal end of the main shaft 110, e.g., to form fluid-tight seals around the outer diameters of the shaft 110, allowing inflation fluid to pressurize the interior volume of the balloon 107 and the expandable membrane to expand radially outward between the proximal and distal ends of the expandable membrane upon the introduction of the inflation fluid.

In some embodiments, the balloon 107 may have a generally toroidal configuration, as schematically illustrated in FIG. 2B, in which the expandable membrane of the balloon 107 has an outer surface and an inner surface, the inner surface forming a closed circumference defining a central hole through which a secondary shaft 114 may extend. The balloon 107 may define an annular interior volume configured to be pressurized by introduction of inflation fluid from the inflation lumen 113. In some embodiments, the balloon 107 may be coupled to the distal end of the main shaft 110 such that it is in fluid communication with the annular shaped lumen 112 as described with respect to FIG. 2A. In some embodiments, the balloon 107 may be coupled to an outer circumference of the main shaft 110 and in fluid communication with an inflation port formed in the sidewall of the main shaft 110, as described elsewhere herein. In some embodiments, the inner surface of the expandable membrane of the balloon 107 may be coupled to (for example, adhered via an adhesive) an outer diameter of the main shaft 110, the secondary shaft 114, and/or another component of the delivery catheter 100. The balloon of FIG. 2B is suitable for use in a balloon angioplasty or balloon valvuloplasty, or can be adapted to support an implantable stent or an implantable or replacement mitral valve or in TAVR or TAVI.

In various embodiments, the delivery catheter may combine or interchange the various features illustrated and/or described with respect to FIGS. 2A-2B.

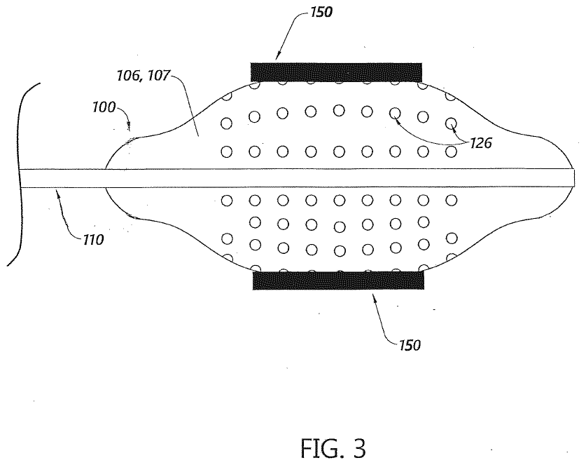

FIG. 3 depicts a delivery catheter 100 having a shaft 110 and an expandable member (balloon) 106,107 with a plurality of pores 126, the expandable member (balloon) 106,107 in an inflated form supporting a stent 150. After the stent is positioned in a vessel to be treated, the expandable member (balloon) 106,107 is deflated and the delivery catheter removed, leaving the stent or replacement valve 150 in place. In the case of implantation, after the stent or valve, e.g., in TAVI, is positioned in the vessel and deployed, the expandable member (balloon) 106,107 is deflated and the delivery catheter removed, leaving the stent or replacement valve 150 in place. In the case of mitral valve replacement or TAVR, after the replacement valve is positioned in surgical site of the removed native mitral valve or aortic valve, the expandable member (balloon) 106,107 is deflated and the delivery catheter removed, leaving the replacement valve 150 in place. In the case of mitral valve implantation, after the implantable valve is positioned in the native mitral valve and deployed, the expandable member (balloon) 106,107 is deflated and the delivery catheter removed, leaving the implantable valve 150 in place. The balloon of FIG. 3 is also suitable for use in a balloon angioplasty or balloon valvuloplasty by omitting the stent or the implantable or replacement mitral valve supported thereon.

In some embodiments, the balloon 107 may be configured to occlude blood flow (for example, upstream or retrograde blood flow) when in an expanded configuration. In some embodiments, the balloon 107 may be configured to displace blood from the target site. Displacing blood from the target site may improve the efficacy of delivering therapeutic agent to the target site (e.g., through the balloon 107). For instance, the therapeutic agent will not be diluted or will be less diluted by blood within the target site. The expandable membrane of the balloon 107 may be sufficiently compliant or conformable to assume the shape of and occlude the target vasculature. In some embodiments, the balloon 107 may be non-compliant (for example, a bag member having a membrane enclosing an expandable interior volume).

In some embodiments, the balloon 107 may be configured to deliver a therapeutic agent, such as a PGG solution, to the target site (e.g., mitral valve or an implantation or surgical site, e.g., in TAVR or TAVI). The balloon 107 may comprise a plurality of pores 126 disposed in the expandable membrane of the balloon configured to place the interior volume of the balloon 107 in fluid communication with the environment of the target site. The solution of therapeutic agent may be used as the inflation fluid. The pores 126 may be configured to provide fluid communication between the interior volume of the balloon 107 and the environment of the target site while allowing for pressurization and inflation of the balloon 107. In some embodiments, the size of the pores 126 may increase as the expandable membrane of the balloon expands. The elastic properties of the expandable membrane of the balloon 107 may allow for a continuous expansion of the pore size of the pores 126 as the interior volume of the balloon 107 is increased causing the expandable membrane to stretch. The volumetric flow rate at which the inflation fluid escapes from the interior volume of the balloon 107 into the environment of the target site may increase as the balloon 107 expands. In some embodiments, the pores 126 may allow for a constant or substantially constant volumetric flow rate of fluid across the pores 126 over a range of pressures of the interior volume. The volumetric flow rate out of the balloon 107 may be maximized at a certain level of pressurization or volumetric flow rates of inflation fluid into the balloon 107. The inflation fluid may be introduced into the interior volume of the balloon 107 at a volumetric flow rate that is greater than the volumetric flow rate at which the inflation fluid flows through the pores 126, such that the balloon 107 may be inflated even while fluid escapes or leaks through the pores 126. In some implementations, the balloon 107 may be inflated using an inflation fluid (for example, saline) that does not comprise the therapeutic agent. The inflation fluid may be switched over to the therapeutic solution or the therapeutic agent may be added to the inflation fluid after the balloon has been inflated. Staggering the delivery of the therapeutic agent may conserve the therapeutic agent and/or may prevent, reduce, or minimize the amount of therapeutic agent that is released into the blood stream before the fluid seal is fully formed.

The pores 126 of the balloon 107 may be disposed uniformly across the surface or a portion of the surface of the balloon 107. In some embodiments, the pores 126 may be disposed in a central portion of the balloon 107 relative to the longitudinal axis. For example, in some embodiments, the length of the balloon 107 may be configured such that the balloon 107 spans the entire length of, e.g., the treatment site or a supported stent 150, and may create a sealed space within the treatment site (not illustrated) when the balloon 107 is expanded to a minimal diameter, as illustrated in FIG. 3. The balloon 107 may form a fluid seal with the vessel. In some embodiments, the balloon 107 may be compliant enough to conform to the shape of the vessel or treatment site. In some embodiments, the expanded balloon 107 may somewhat expand the vessel or treatment site or the mitral valve or implantation or surgical site. e.g., TAVR or TAVI. When the balloon 107 is expanded, the counter pressure of the vessel or treatment site against the outer diameter of the balloon 107 may effectively seal the pores 126 from the intravascular environment such that fluid may not flow at any substantial flow rate through those pores 126. This configuration may prevent or minimize delivery of therapeutic agent into non-targeted volumes of the vessel or treatment site (e.g., the mitral valve or implantation or surgical site). In some embodiments, contact between the therapeutic agent within the inflation fluid with the tissue sealed against the pores 126 may be used to treat the vessel or treatment site. In some embodiments a plurality of the pores 126 may be spaced at a high density over an area configured to be pressed into contact with the vessel or treatment site. In some embodiments, the pores 126 may be brought into close proximity (for example, no more than 0.3 mm, 0.2 mm, 0.1 mm, 0.075 mm 0.05 mm, 0.025 mm, 0.001 mm, etc.) to the vessel or treatment site but not into substantial contact.

In some embodiments, one or more of the components of the delivery catheter 100 may comprise radiopaque materials or radiopaque elements (for example, radiopaque rings) may be added to the delivery catheter 100. For example, radiopaque rings may be added to one or more of the distal end of the main shaft 110, the distal end of the secondary shaft 114, the distal and/or proximal ends of the intermediate shaft segment 120, the expandable member 106,107, and/or the balloon 107 (for example, at proximal and/or distal ends of the balloon). Use of radiopaque elements or other detectable elements may allow for visual tracking of the delivery catheter within the vasculature, such as through radioscopy or other suitable imaging means, and/or may allow for evaluation of the positioning of the balloon 107 within the vasculature. In some implementations, the inflation fluid of the balloon 107 may include a contrast agent. Use of the contrast agent may allow the user to evaluate the state or amount of inflation of the balloon, may allow the user to determine if the balloon has occluded the vessel or treatment site, and/or, in the case of the balloon 107, may allow the user to monitor the delivery of the therapeutic agent into the vessel or treatment area (e.g., the mitral valve or implantation or surgical site, e.g., in TAVR or TAVI).

In some embodiments, the delivery catheter 100 may be useable with one or more guidewires for facilitating the introduction and/or navigation of the device into and within the vasculature. In some embodiments, a guidewire may be received within the first central lumen 112, such as when the secondary shaft 114 is removable from the first central lumen 112. In some embodiments, the lumen may be configured to prevent a guidewire from extending distally beyond a certain point along the length of the lumen. For example, the secondary lumen may be dimensioned with a catch or a tapered or step-down in diameter that prevents the guidewire from extending distally any further. The guidewire may be configured to extend distally beyond the distal end of the secondary shaft 114 in embodiments where the central lumen is open distally to the intravascular environment. In some implementations, the delivery catheter 100 may be introduced over the guidewire after the guidewire has been navigated to or near the target site. In some implementations, the delivery catheter 100 may be capable of being navigated to the target site without use of a guidewire. For example, the delivery catheter 100 may be readily pushed into position via access through the femoral artery without the need for steerability. In some embodiments, the delivery catheter 100 may comprise steerable components, such as the main shaft 110, which may be configured to bend near a distal end of the device. The delivery catheter 100 may comprise one or more pull wires which extend from or from near a distal end of the device to a proximal end of the device. Operation of a control on the proximal end of the delivery catheter 100 may be configured to bend a distal portion of the delivery catheter 100 in one or more directions. Steerability of the delivery catheter 100 may facilitate the introduction and/or navigation of the delivery catheter 100.

In some embodiments, the lumens described elsewhere herein may not be formed from the concentric positioning of two or more shafts, but rather may be configured as internal lumens formed as channels within the bodies of one or more unitary shafts. For example, the main shaft 110 may extend from a proximal end of the device, through a center of the balloon 107. The main shaft 110 may comprise a plurality of internal lumens (for example, non-concentric lumens) formed within the body material of the main shaft 110. The internal lumens may run substantially parallel to one another. The internal lumens may extend to different lengths along the longitudinal axis of the delivery catheter 100. The internal lumens may be in fluid communication with different components of the delivery catheter 100. For example, the internal lumen may be in fluid communication with the balloon 107. The main shaft 110 or other shaft components may comprise additional lumens beyond what is described elsewhere herein. For example, the delivery catheter 100 may have lumens configured for receiving guidewires and/or lumens configured for providing aspiration.

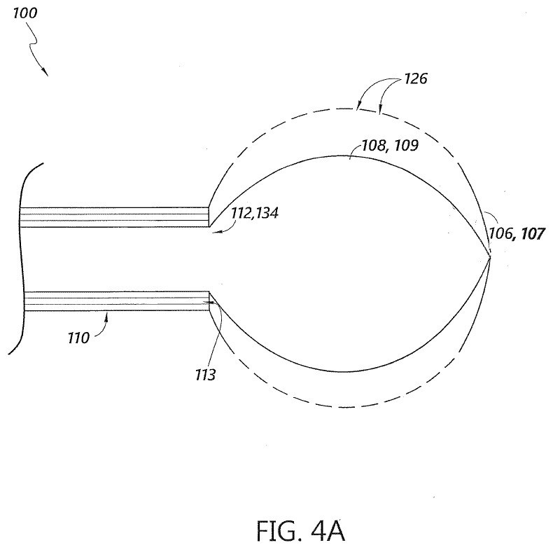

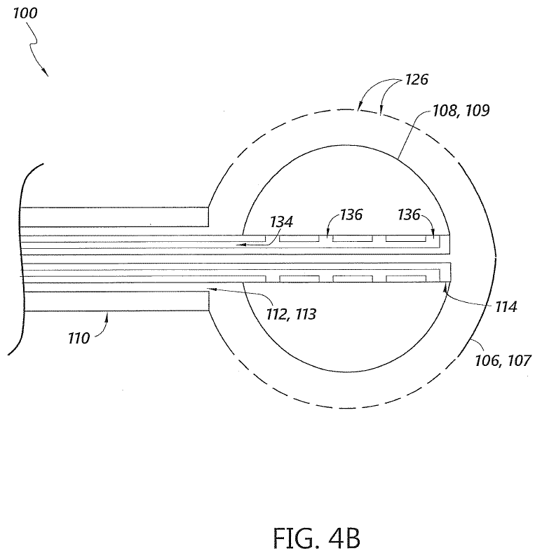

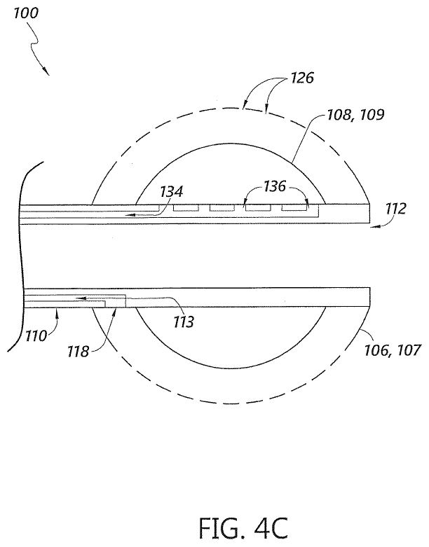

FIGS. 4A-4C schematically illustrate examples of a delivery catheter 100 comprising a second expandable member 108,109. The balloons of FIG. 4A-4C are suitable for use in a balloon angioplasty or balloon valvuloplasty, or can be adapted to support a stent or an implantable or replacement mitral valve. The second expandable member 108 may be an inner balloon 109 as shown in FIG. 4A. FIGS. 4A and 4B may comprise features that are the same or relatively similar to those described with respect to FIG. 2A. The inner balloon 109 may be positioned entirely within the interior of the balloon 105 as shown in FIGS. 4A-4C. The inner balloon 109 may be in fluid communication with a tertiary inflation lumen 134. As shown in FIG. 4A, the tertiary inflation lumen 134 may be formed within the main shaft 110. In some embodiments, the tertiary inflation lumen 134 may be formed radially inside the first inflation lumen 113. The tertiary inflation lumen 134, may be formed by the first central lumen 112, as shown in FIG. 4A. In some embodiments, the tertiary inflation lumen 134 may be formed from a separate tubular component that is carried within the first central lumen 112 of the main shaft 110.

The inner balloon 109 may comprise an expandable membrane. The expandable membrane of the inner balloon 109 may comprise the same and/or different material(s) as the expandable membrane of the balloon 107. In some embodiments, such as that shown in FIG. 4A, the expandable membrane is coupled to (for example, at or near) the secondary shaft 114 forming a fluid tight seal with the secondary shaft 114 such that an interior volume of the inner balloon 109 may be pressurized. Introduction of inflation fluid into the upstream balloon 105 may cause the inner balloon 109 to expand radially outward between the tertiary inflation lumen 134 and the distal fluid tight seal. The distal end of the expandable membrane of the inner balloon 109 may be substantially longitudinally aligned with the distal end of the expandable membrane of the balloon 107 or may be coupled to the secondary shaft 114 at a point proximal to that where the expandable membrane of the balloon 107 is coupled to the secondary shaft 114.

In some embodiments, as shown in FIG. 4B, proximal and distal ends of the expandable membrane of the inner balloon 109 may be coupled to the secondary shaft 114 to form fluid-tight seals around the outer diameter of the secondary shaft 114. The distal end of the expandable membrane of the inner balloon 109 may be substantially longitudinally aligned with the distal end of the expandable membrane of the balloon 107 or may be coupled to the secondary shaft 114 at a point proximal to that where the expandable membrane of the balloon 107 is coupled to the secondary shaft 114. The proximal end of the expandable membrane of the inner balloon 109 may be substantially longitudinally aligned with the proximal end of the expandable membrane of the balloon 107. Inflation fluid may be introduced to pressurize the interior volume of the inner balloon 109 allowing the expandable membrane to expand radially outward between the proximal and distal ends of the expandable membrane of the inner balloon 109 upon the introduction of the inflation fluid. Inflation fluid may be introduced into the interior of the inner balloon 109 through one or more tertiary inflation ports 136 formed in the sidewall of the secondary shaft 114. The tertiary inflation lumen 134 may be disposed within the secondary shaft 114 rather than the main shaft 110. The tertiary inflation ports 136 may pass through a sidewall of the secondary shaft 114. In some embodiments, a plurality of tertiary inflation ports 136 may be spaced longitudinally along the secondary shaft 114 between the proximal and distal ends of the expandable membrane of the inner balloon 109. In some embodiments, a plurality of tertiary inflation ports 136 may be spaced radially around the outer diameter of the secondary shaft 114.

In some embodiments, as shown in FIG. 4C, the tertiary inflation ports 136 are formed in a sidewall of the main shaft 110 and the inner balloon 109 may be coupled at proximal and distal sealing points to an outer diameter of the main shaft 110. In some embodiments, the inner balloon 109 may be a generally toroidal balloon, as described elsewhere herein with respect to balloon 107. The toroidal inner balloon 109 may be disposed within the interior volume of the balloon 107, and an inflation port 118 is in fluid communication with the interior volume of the balloon 107. In some embodiments, the inner surface of the expandable membrane of the toroidal inner balloon 109 may be coupled at a proximal end, distal end, or along a length or portions of the length of the inner surface to the main shaft 110. In some embodiments, the inner toroidal balloon 109 may be coupled to the expandable membrane of the balloon 107. In some embodiments, the inner toroidal balloon 109 may be coupled to a shaft and the expandable membrane of the balloon 107. In some embodiments, the toroidal inner balloon 109 may be free-floating within the interior volume of the balloon 107. In some embodiments, the balloon 107 may be a generally toroidal balloon as described elsewhere herein and the inner balloon 109 may be disposed within the annular interior volume of the balloon 107. The generally toroidal inner balloon 109 may be coupled to an inner surface and/or an outer surface of the expandable membrane of the generally toroidal balloon 107 or the inner balloon 109 may be free-floating within the annular interior volume of the balloon 107.

The inner balloon 109 may facilitate the expansion of the balloon 107 and/or the expulsion of inflation fluid (including therapeutic agent) from the balloon 107. The inclusion and inflation of an inner balloon 109 may advantageously reduce the volume of inflation fluid within the balloon 107 necessary to expand the balloon and/or expel inflation fluid through the pores 126 of the balloon 107. The reduction of inflation fluid used within the inner balloon 109 may conserve the therapeutic agent. The use of the inner balloon 109 may reduce the pressure within the interior of the balloon 107 at which inflation fluid is expelled through the pores 126. In some implementations, a volume of inflation fluid may be introduced into the interior volume of the balloon 107 which is insufficient to fully expand the balloon 107 or to expand the balloon 107 to the inner diameter of the vessel or treatment site. The inner balloon 109 may be inflated, pressing the volume of inflation fluid within the interior of the balloon 107 against the expandable membrane of the balloon 107 and causing the balloon 107 to expand. In some embodiments, the volume of inflation fluid may be delivered through the pores 126 at a substantial (for example, non-negligible) rate as soon as the combined volume of the inner balloon 109 and the volume of inflation fluid within the balloon 107 is substantially equal to the interior volume of the balloon 107 or as soon as the reduction of volume available for the volume of inflation fluid is small enough that it causes the internal pressure within the balloon 107 to surpass a minimum threshold.

Any or all of the balloons described herein may comprise various shapes. The shapes of the device balloons may be the same or different. In various embodiments, the shape of the balloon may be defined by a surface of revolution. In some embodiments, the balloons may comprise a substantially spherical shape. In some embodiments, the balloons may comprise a spheroid shape, such as a prolate spheroid shape or an oblate spheroid shape. The longitudinal axis of the spheroid may be aligned with the longitudinal axis of the delivery catheter 100. In various embodiments, the length of the balloon may be larger than a diameter of the balloon in its expanded configuration (for example, a prolate spheroid). In some embodiments, the balloons may comprise a pointed football shape. In some embodiments, the balloons may comprise a cylindrical shape. The balloons may comprise distinct proximal and distal surfaces extending from the longitudinal axis of the delivery device 100 to form an edge with an outer surface of the balloon. The proximal and/or distal surfaces may be substantially flat, generally concave, and/or generally convex. The outer surface of the balloons may extend to a diameter greater than, substantially equal to, or less than a diameter of the proximal surface and/or the distal surface. The outer surface may be generally flat, concave, or convex. In some embodiments the pores 126 of the weeping balloon may be only disposed on the outer surface of the balloon or on an outer surface and only one of the proximal and distal surfaces (for example, the distal surface of the balloon 107). In some embodiments, the balloon 107 may comprise one or more inner layers including inner pores. In some embodiments, the inner pores may generally comprise diameters greater than or equal to the diameter of the pores 126. The inner pores may serve as baffles which may help facilitate uniform distribution of the inflation fluid (and therapeutic agent) within the interior of the balloon 107.

The outer diameter of the balloon 107 in an expanded configuration (for example, at its widest point) may be sized to a diameter of at least approximately 1.5 cm, 1.75 cm, 2.0 cm, 2.25 cm, 2.5 cm, 3.0 cm, 3.5 cm, or 4.0 cm or more. The outer diameter of the balloon 107 in an expanded configuration may be configured to match or slightly exceed the diameter of a healthy vessel or a healthy mitral valve. In some embodiments, the balloon 107 may be configured to expand to the diameter of a healthy vessel or a healthy mitral valve or slightly exceed the diameter of a healthy vessel or a healthy mitral valve such that it may form a fluid seal downstream and/or upstream of the vessel or treatment site. In some embodiments, the total volume of the balloon 107 (for example, in an expanded configuration) or of the holding capacity of deliverable fluid of the delivery catheter 100 (for example, the interior volume of the balloon 107 and the inflation lumen 113) may be at least about 1 mL or less, 2 mL, 3 mL, 5 mL, 10 mL, 20 mL, 30 mL, 40 mL, 50 mL, 60 mL, 70 mL, 80 mL, 90 mL, 100 mL, 125 mL, 150 mL, 175 mL, or 200 mL or more.

The length of the balloon 107 may be at least about 0.5 cm, 1 cm, 2 cm, 3 cm, 4 cm, 5 cm, 6 cm, 7 cm, 8 cm, 9 cm, or 10 cm. In some embodiments, the length of the balloon 107 may be configured to accommodate a supported stent or a supported implantable or replacement mitral valve or valve in a TAVR or TAVI.

In embodiments comprising an inner balloon 109, the inner balloon 109 may be the same or a different shape as the balloon 107. The inner balloon 109 may comprise an expanded diameter the same as or less than that of the balloon 107. The inner balloon 109 may comprise a length the same as or less than that of the balloon 107. The inner balloon 109 may comprise a maximum interior volume the same as or less than that of the balloon 107. In some embodiments, the volume, length, and/or expanded diameter of the inner balloon 109 may be no less than approximately 100%, 95%, 90%, 85%, 80%, 75%, 70%, 65%, 60%, 55%, 50%, 45%, or 40% of the balloon 107. In embodiments, in which the length of the inner balloon 109 is less than the length of the balloon 107, the inner balloon 109 may be positioned, with respect to the longitudinal axis, centrally within the balloon, or toward the proximal or distal end of the balloon 107. The proximal end of the inner balloon 109 may or may not be aligned with the proximal end of the balloon 107. The distal end of the inner balloon 109 may or may not be aligned with the distal end of the balloon 107.

In some embodiments, the unexpanded diameters of the balloon 107, and/or the inner balloon 109 of the delivery catheter 100 may be no greater than about 0.5 mm, 1 mm, 2 mm, 3 mm, 4 mm, 5 mm, 6 mm, 7 mm, 8 mm, 9 mm, or 10 mm. The unexpanded diameter of one or more of the balloons may be configured to be received within the lumen of a concentrically surrounding shaft or access sheath.

In some embodiments the weeping balloon (for example, balloon 107) may comprise at least 5, 10, 20, 30, 40, 50, 100, 200, 300, 500, or 1000 pores 126. The diameter (or longest dimension) of the individual pores 126 may be the same or may be different. The diameter of the pores 126 (for example, in an expanded configuration) may be no greater than approximately 0.01 mm, 0.02 mm, 0.03 mm, 0.05 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, or 1 mm. In some embodiments, the diameter of the pores 126 in the expanded configuration may be at least about 1×, 1.25×, 1.5×, 1.75×, 2×, 3×, 4×, 5×, or 10×, the diameter of the pores 126 in the unexpanded configuration. The pores 126 may be the same size regardless the state of expansion in some embodiments, particularly if balloon 107 comprises a non-compliant expandable membrane. In some embodiments, the pores 126 may be disposed over an entire length of the balloon 107. In some embodiments, the pores 126 may be disposed over only about the middle 20%, 30%, 40%, 50%, 60%, 70%, 80%, 90% or 95% of the length of the balloon 107 (for example, in an expanded configuration). In some embodiments, the pores 126 may be disposed only over a distal portion of the length of the balloon 107, the distal portion comprising no more than about 20%, 30%, 40%, 50%, 60%, 70%, 80%, 90% or 95% of the length of the balloon 107 (for example, in an expanded configuration).

In some embodiments, the outer diameter of the main shaft 110 may be no greater than about 1 mm, 2 mm, 3 mm, 4 mm, 5 mm, 6 mm, 7 mm, 8 mm, 9 mm, or 10 mm. In some embodiments, the outer diameter of the main shaft 110 may be approximately 9 Fr, 10 Fr, 11 Fr, 12 Fr, 13 Fr, 14 Fr, 15 Fr, 16 Fr 17 Fr, or 18 Fr. The main shaft 110 may have a sidewall thickness of no greater than approximately 0.1 mm, 0.2 mm, 0.3 mm, 0.4 mm, 0.5 mm, 0.6 mm, 0.7 mm, 0.8 mm, 0.9 mm, 1 mm, 1.25 mm, 1.5 mm, 1.75 mm, or 2.0 mm. The secondary shaft 114 may comprise an outer diameter substantially equal to or slightly less than the inner diameter of the main shaft 110. In some embodiments, the length of the delivery catheter 100 from its proximal end to its distal end 102 may be at least about 20 cm, 25 cm, 30 cm, 35 cm, 40 cm, 45 cm, or 50 cm.

The various components of the delivery catheter 100 may be fabricated from one or more materials known in the art of catheter design. The materials, particularly those configured to be placed in contact with the intravascular environment, may be fabricated from biocompatible materials. In some embodiments, one or more components of the delivery catheter, such as the main shaft 110 and/or secondary shaft 114, may comprise polyurethane (PU), polyethylene (PE), polyvinylchloride (PVC), polytetrafluoroethylene (PTFE), polyvinylidene fluoride (PVDF), other fluoropolymers, polyether block amide (for example, PEBAX® or Vestamid®), nylon, etc. In various embodiments, the shafts and/or balloons may be chemically and/or mechanically treated/processed (for example, plasma etched) or coated to provide biocompatibility or mechanical properties (for example, lubricious and/or hydrophilic surface properties). For example, one or more components of the delivery catheter 100 may be coated with a formulation comprising polyethylene glycol (PEG).

In some embodiments, the delivery catheter 100 may comprise a handle at its proximal end. The main shaft 110 of the delivery catheter 100 may extend from a distal end of the handle. The main shaft 110 may continue through the handle and/or be in fluid communication with a channel formed within the handle. The handle may comprise a grip portion for the operator to grasp. The handle may be used to distally advance and/or proximally retract the delivery catheter 100. In embodiments where the delivery catheter 100 is steerable, the handle may comprise one or more controls for steering (for example, bending a distal portion of) the delivery catheter 100, such as by controlling the extension and retraction of one or more pull wires. In some embodiments, the handle may comprise one or more fluid ports in fluid communication with one or more of the internal lumens, such as the first inflation lumen 113 and the secondary inflation lumen 117. The fluid ports may comprise luer-type connectors for connecting to fluid lines, such as for supplying inflation fluid to the delivery catheter 100. In some embodiments, the fluid ports may comprise stopcocks or other valves for regulating fluid flow from a fluid supply source into the handle. The fluid lines may extend to sources of pressurized fluid (for example, inflation fluid) such as a syringe or pump and/or a vacuum source for providing aspiration. In some embodiments, one more fluid ports may be configured to receive a component of the delivery catheter 100. For example, in embodiments, in which the secondary shaft 114 is removable from the main shaft 110, the secondary shaft 114 may be insertable into a proximal end of the handle through the fluid port to be received in the main shaft 110. The secondary shaft 114 may be advanced through the fluid port until it extends distally beyond the main shaft 110. The handle may temporarily fix the relative positioning of the shafts 110, 114, as described elsewhere herein. Similarly, in some embodiments, a guidewire may be insertable into a proximal end of the handle through one or more fluid ports to be received in the first central lumen 112 or the secondary central lumen 116. In some embodiments, in which inflation fluid is supplied by a pump or mechanized syringe, and/or in which aspiration is provided, there may be a controller for controlling flow rate through the internal lumens. The controller may be remote to the handle or coupled to or integral with the handle. The handle may comprise one or more controls for modulating (for example, increasing, decreasing, stopping, and/or starting) the flow rate of the inflation fluid and/or the vacuum pressure supplied to one or more of the internal lumens. In some embodiments, the controls may be remote from the handle (for example, part of a remote controller).

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods that are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

The term “Solvate” as used herein is a broad term, and is to be given its ordinary and customary meaning to a person of ordinary skill in the art (and is not to be limited to a special or customized meaning), and refers without limitation to the compound formed by the interaction of a solvent and a compound described herein or salt thereof. Suitable solvates are pharmaceutically acceptable solvates including hydrates.

The term “pharmaceutically acceptable salt” as used herein is a broad term, and is to be given its ordinary and customary meaning to a person of ordinary skill in the art (and is not to be limited to a special or customized meaning), and refers without limitation to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, (CH₃)₂CHCH₂— CH₃CH₂CH(CH₃)— and (CH₃)₃C—.

The term “halogen” or “halo,” as used herein, is a broad term, and is to be given its ordinary and customary meaning to a person of ordinary skill in the art (and is not to be limited to a special or customized meaning), and refers without limitation to any one of the radio-stable atoms of column 7 of the Periodic Table of the Elements, for example, fluorine, chlorine, bromine, or iodine.

As used herein, “alkyl” is a broad term, and is to be given its ordinary and customary meaning to a person of ordinary skill in the art (and is not to be limited to a special or customized meaning), and refers without limitation to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; for example, “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” is a broad term, and is to be given its ordinary and customary meaning to a person of ordinary skill in the art (and is not to be limited to a special or customized meaning), and refers without limitation to the alkyl moiety substituted with at least one halo group. Examples of haloalkyl groups include, but are not limited to, —CF₃, —CHF₂, —CH₂F, —CH₂CF₃, —CH₂CHF₂, —CH₂CH₂F, —CH₂CH₂Cl, or —CH₂CF₂CF₃.

As used herein, “alkoxy” is a broad term, and is to be given its ordinary and customary meaning to a person of ordinary skill in the art (and is not to be limited to a special or customized meaning), and refers without limitation to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” is a broad term, and is to be given its ordinary and customary meaning to a person of ordinary skill in the art (and is not to be limited to a special or customized meaning), and refers without limitation to the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” is a broad term, and is to be given its ordinary and customary meaning to a person of ordinary skill in the art (and is not to be limited to a special or customized meaning), and refers without limitation to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” is a broad term, and is to be given its ordinary and customary meaning to a person of ordinary skill in the art (and is not to be limited to a special or customized meaning), and refers without limitation to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

The term “aromatic” as used herein is a broad term, and is to be given its ordinary and customary meaning to a person of ordinary skill in the art (and is not to be limited to a special or customized meaning), and refers without limitation to a ring or ring system having a conjugated pi electron system and includes both carbocyclic aromatic (for example, phenyl) and heterocyclic aromatic groups (for example, pyridine). The term includes monocyclic or fused-ring polycyclic (i.e., rings which share adjacent pairs of atoms) groups provided that the entire ring system is aromatic.

As used herein, “aryl” is a broad term, and is to be given its ordinary and customary meaning to a person of ordinary skill in the art (and is not to be limited to a special or customized meaning), and refers without limitation to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” are broad terms, and are to be given their ordinary and customary meaning to a person of ordinary skill in the art (and are not to be limited to a special or customized meaning), and refer without limitation to RO— and RS—, in which R is an aryl as is defined above, such as “C₆₋₁₀ aryloxy” or “C₆₋₁₀ arylthio” and the like, including but not limited to phenyloxy.

As used herein, “aralkyl” or “arylalkyl” are broad terms, and are to be given their ordinary and customary meaning to a person of ordinary skill in the art (and are not to be limited to a special or customized meaning), and refer without limitation to an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “alkylene” is a broad term, and is to be given its ordinary and customary meaning to a person of ordinary skill in the art (and is not to be limited to a special or customized meaning), and refers without limitation to a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “heteroaryl” is a broad term, and is to be given its ordinary and customary meaning to a person of ordinary skill in the art (and is not to be limited to a special or customized meaning), and refers without limitation to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members including one or more nitrogen, oxygen and sulfur in the ring backbone. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “heteroaralkyl” or “heteroarylalkyl” are broad terms, and are to be given their ordinary and customary meaning to a person of ordinary skill in the art (and are not to be limited to a special or customized meaning), and refer without limitation to a heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” is a broad term, and is to be given its ordinary and customary meaning to a person of ordinary skill in the art (and is not to be limited to a special or customized meaning), and refers without limitation to a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” is a broad term, and is to be given its ordinary and customary meaning to a person of ordinary skill in the art (and is not to be limited to a special or customized meaning), and refers without limitation to a fully saturated carbocyclyl ring or ring system. Examples include cyclopropyl, cyclobutyl, cyclopentyl, and cyclohexyl.

As used herein, “cycloalkenyl” is a broad term, and is to be given its ordinary and customary meaning to a person of ordinary skill in the art (and is not to be limited to a special or customized meaning), and refers without limitation to a carbocyclyl ring or ring system having at least one double bond, wherein no ring in the ring system is aromatic. An example is cyclohexenyl.

As used herein, “heterocyclyl” is a broad term, and is to be given its ordinary and customary meaning to a person of ordinary skill in the art (and is not to be limited to a special or customized meaning), and refers without limitation to a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “acyl” is a broad term, and is to be given its ordinary and customary meaning to a person of ordinary skill in the art (and is not to be limited to a special or customized meaning), and refers without limitation to —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. Non-limiting examples include formyl, acetyl, propanoyl, benzoyl, and acryl.

As used herein, “O-carboxy” group is a broad term, and is to be given its ordinary and customary meaning to a person of ordinary skill in the art (and is not to be limited to a special or customized meaning), and refers without limitation to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “C-carboxy” group is a broad term, and is to be given its ordinary and customary meaning to a person of ordinary skill in the art (and is not to be limited to a special or customized meaning), and refers without limitation to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes carboxyl (i.e., —C(═O)OH).

As used herein, “cyano” group is a broad term, and is to be given its ordinary and customary meaning to a person of ordinary skill in the art (and is not to be limited to a special or customized meaning), and refers without limitation to a “—CN” group.

As used herein, “cyanato” group is a broad term, and is to be given its ordinary and customary meaning to a person of ordinary skill in the art (and is not to be limited to a special or customized meaning), and refers without limitation to an “—OCN” group.

As used herein, “isocyanato” group is a broad term, and is to be given its ordinary and customary meaning to a person of ordinary skill in the art (and is not to be limited to a special or customized meaning), and refers without limitation to a “—NCO” group.

As used herein, “thiocyanato” group is a broad term, and is to be given its ordinary and customary meaning to a person of ordinary skill in the art (and is not to be limited to a special or customized meaning), and refers without limitation to a “—SCN” group.

As used herein, “isothiocyanato” group is a broad term, and is to be given its ordinary and customary meaning to a person of ordinary skill in the art (and is not to be limited to a special or customized meaning), and refers without limitation to an “—NCS” group.

As used herein, “sulfinyl” group is a broad term, and is to be given its ordinary and customary meaning to a person of ordinary skill in the art (and is not to be limited to a special or customized meaning), and refers without limitation to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “sulfonyl” group is a broad term, and is to be given its ordinary and customary meaning to a person of ordinary skill in the art (and is not to be limited to a special or customized meaning), and refers without limitation to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “S-sulfonamido” group is a broad term, and is to be given its ordinary and customary meaning to a person of ordinary skill in the art (and is not to be limited to a special or customized meaning), and refers without limitation to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “N-sulfonamido” group is a broad term, and is to be given its ordinary and customary meaning to a person of ordinary skill in the art (and is not to be limited to a special or customized meaning), and refers without limitation to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “O-carbamyl” group is a broad term, and is to be given its ordinary and customary meaning to a person of ordinary skill in the art (and is not to be limited to a special or customized meaning), and refers without limitation to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “N-carbamyl” group is a broad term, and is to be given its ordinary and customary meaning to a person of ordinary skill in the art (and is not to be limited to a special or customized meaning), and refers without limitation to an “—N(R_{A})C(═O)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “O-thiocarbamyl” group is a broad term, and is to be given its ordinary and customary meaning to a person of ordinary skill in the art (and is not to be limited to a special or customized meaning), and refers without limitation to a “—OC(═S)NR_{A}R_{B}” group in which R^{A} and R^{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “N-thiocarbamyl” group is a broad term, and is to be given its ordinary and customary meaning to a person of ordinary skill in the art (and is not to be limited to a special or customized meaning), and refers without limitation to an “—N(R^{A})C(═S)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “C-amido” group is a broad term, and is to be given its ordinary and customary meaning to a person of ordinary skill in the art (and is not to be limited to a special or customized meaning), and refers without limitation to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “N-amido” group is a broad term, and is to be given its ordinary and customary meaning to a person of ordinary skill in the art (and is not to be limited to a special or customized meaning), and refers without limitation to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “amino” group is a broad term, and is to be given its ordinary and customary meaning to a person of ordinary skill in the art (and is not to be limited to a special or customized meaning), and refers without limitation to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes free amino (i.e., —NH₂).

As used herein, “aminoalkyl” group is a broad term, and is to be given its ordinary and customary meaning to a person of ordinary skill in the art (and is not to be limited to a special or customized meaning), and refers without limitation to an amino group connected via an alkylene group.

As used herein, “alkoxyalkyl” group is a broad term, and is to be given its ordinary and customary meaning to a person of ordinary skill in the art (and is not to be limited to a special or customized meaning), and refers without limitation to an alkoxy group connected via an alkylene group, such as a “C₂₋₈ alkoxyalkyl” and the like.

As used herein, “haloalkoxy” refers to the formula —OR wherein R is a haloalkyl as defined above, such as —CF₃, —CHF₂, —CH₂F, —CH₂CF₃, —CH₂CHF₂, —CH₂CH₂F, —CH₂CH₂Cl, or —CH₂CF₂CF₃.

As used herein, the term “substituted”, as in a substituted group, is a broad term, and is to be given its ordinary and customary meaning to a person of ordinary skill in the art (and is not to be limited to a special or customized meaning), and refers without limitation to a group that is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₂-C₆ alkenyl, C₂-C₆ alkynyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (for example, —CF₃), halo(C₁-C₆)alkoxy (for example, —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O).

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene.”

When two R groups are said to form a ring (for example, a heterocyclyl, or heteroaryl ring) “together with the atom to which they are attached,” it is meant that the collective unit of the atom and the two R groups are the recited ring. The ring is not otherwise limited by the definition of each R group when taken individually.

Similarly, when two “adjacent” R groups are said to form a ring “together with the atoms to which they are attached,” it is meant that the collective unit of the atoms, intervening bonds, and the two R groups are the recited ring. For example, when the following substructure is present:

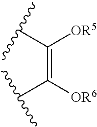

and R⁵ and R⁶ are defined as hydrogen or R^{A}, where adjacent R^{A} together with the atoms to which they are attached form a heterocyclyl, or heteroaryl ring, it is meant that R⁵ and R⁶ can be selected from hydrogen or R^{A}, or alternatively, the substructure has structure:

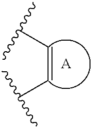

where A is a heterocyclyl, or heteroaryl ring containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

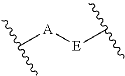

includes the substituent being oriented such that the A is attached at the leftmost attachment point of the molecule as well as the case in which A is attached at the rightmost attachment point of the molecule.

The term “Subject” as used herein, is a broad term, and is to be given its ordinary and customary meaning to a person of ordinary skill in the art (and is not to be limited to a special or customized meaning), and refers without limitation to a human or a non-human mammal, for example, a dog, a cat, a mouse, a rat, a cow, a sheep, a pig, a goat, a non-human primate or a bird, for example, a chicken, as well as any other vertebrate or invertebrate.

The term “mammal” as used herein is a broad term, and is to be given its ordinary and customary meaning to a person of ordinary skill in the art (and is not to be limited to a special or customized meaning), and is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

An “effective amount” or a “therapeutically effective amount” are broad terms, and are to be given their ordinary and customary meaning to a person of ordinary skill in the art (and are not to be limited to a special or customized meaning), and refer without limitation to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein are broad terms, and are to be given their ordinary and customary meaning to a person of ordinary skill in the art (and are not to be limited to a special or customized meaning), and refer without limitation to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

Some embodiments of include methods of treating peripheral vascular disease with compositions comprising PGG as described herein. In some embodiments, a subject can be a mammal, e.g., a human. In some embodiments, the mammal can be a dog, cat, horse, rabbit, goat, sheep or other mammal.

Further embodiments include administering a combination of compounds to a subject in need thereof. A combination can include PGG with an additional medicament.

Some embodiments include co-administering PGG or a composition containing PGG with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered intravenously.

Examples of additional medicaments include collagen crosslinking agents, such as glutaraldehyde, genipin acyl azide, and/or epoxyamine. Other additional medicaments include therapeutic agents for treating congestive heart failure, as described elsewhere herein.

To further illustrate, examples are included. The examples should not, of course, be construed as specifically limiting the invention. Variations of these examples within the scope of the claims are within the purview of one skilled in the art and are considered to fall within the scope of the invention as described and claimed herein. One will recognize that the skilled artisan, armed with the present disclosure, and skill in the art is able to prepare and use the devices and compositions without exhaustive examples.

Although the invention has been described with reference to embodiments and examples, it should be understood that numerous and various modifications can be made without departing from the spirit of the invention. Accordingly, the invention is limited only by the following claims.

It is understood that this disclosure, in many respects, is only illustrative of the numerous alternative device embodiments of the present invention. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (for example, compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (for example, where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-15. (canceled)

16. A method for treating stress urinary incontinence in a patient, comprising:
delivering a therapeutic agent to a patient in need thereof, wherein the therapeutic agent comprises pentagalloyl glucose (PGG).

17. The method of claim 16, wherein the PGG is applied to at least one of a tendon or a ligament that supports at least one urinary tract organ.

18. The method of claim 17, wherein the PGG is placed in a pelvic cavity, or in or on a structure selected from the group consisting of a kidney, a ureter, a bladder, a urethra, or a sphincter.

19. The method claim 18, wherein the PGG is provided as a coating or component of an urogynecologic mesh or sling.

20. The method of claim 19, wherein the PGG is provided in admixture with a poloxamer gel exhibiting a reverse thermosensitive property.

21. The method of claim 20, wherein the PGG is substantially free of gallic acid or methyl gallate.

22. The method of claim 21, further comprising at least one additional therapeutic agent selected from the group consisting of oxybutynin, tolterodine, darifenacin, fesoterodine, solifenacin, trospium, and mirabegron.

23. A method for treating pelvic organ prolapse in a patient, comprising:
delivering a therapeutic agent to a patient in need thereof, wherein the therapeutic agent comprises pentagalloyl glucose (PGG).

24. The method of claim 23, wherein the PGG is applied to at least one of a tendon or a ligament that supports at least one pelvic organ.

25. The method of claim 24, wherein the PGG is placed in a pelvic cavity or in or on a pelvic organ.

26. The method of claim 25, wherein the PGG is provided as a coating or component of an urogynecologic mesh.

27. The method of claim 26, wherein the PGG is provided in admixture with a poloxamer gel exhibiting a reverse thermosensitive property.

28. The method of claim 27, wherein the PGG is substantially free of gallic acid or methyl gallate.

29. A method for treating a peripheral vascular disease, selected from the group consisting of peripheral arterial disease, chronic venous insufficiency, deep vein thrombosis, and varicose veins, in a patient, comprising:
delivering a therapeutic agent to a patient in need thereof, wherein the therapeutic agent comprises pentagalloyl glucose (PGG).

30. The method of claim 29, wherein the therapeutic agent is substantially free of gallic acid or methyl gallate.

31. The method of claim 30, wherein the therapeutic agent further comprises one or more pharmaceutically-acceptable excipients.

32. The method of claim 31, further comprising delivering at least one anticoagulant, selected from at least one member of the group consisting of heparin, enoxaparin, dalteparin, fondaparinux, warfarin, dabigatra, rivaroxaban, apixaban, and edoxaban.

33. The method of claim 32, further comprising delivering a thrombolytic agent, selected from at least one member of the group consisting of reteplase, alteplase, urokinase, prourokinase, anisoylated purified streptokinase activator complex), and streptokinase.

34. The method of claim 33, wherein the PGG is administered via a route selected from systemic administration in an oral or intravenous form, topical administration directly to a vessel to be treated or tissue adjacent to the vessel to be treated, injection into a treatment area or a vessel to be treated, or delivery via a catheter into a vessel to be treated.

35. The method of claim 34, wherein the PGG is admixed with a biocompatible poloxamer gel having reverse thermosensitive properties.
